(19) United States
(12) Patent Application Publication
(10) Pub. No.: US 2025/0320273 A1
(43) Pub. Date: Oct. 16, 2025

(54) DECOY CYTOKINE RECEPTOR

(71) Applicants: National University of Singapore, Sinapore (SG); Singapore Health Services PTE LTD., Singapore (SG)

(72) Inventors: Stuart Alexander Cook, Singapore (SG); Sebastian Schaefer, Singapore (SG)

(73) Assignees: National University of Singapore, Singapore (SG); Singapore Health Services PTE LTD., Singapore (SG)

(21) Appl. No.: 19/038,814

(22) Filed: Jan. 28, 2025

Related U.S. Application Data
(62) Division of application No. 17/687,554, filed on Mar. 4, 2022, now Pat. No. 12,240,889.
Application No. 17/687,554 is a division of application No. 16/469,601, filed on Jun. 13, 2019, now Pat. No. 11,274,143.

(30) Foreign Application Priority Data
Dec. 16, 2016 (GB) 1621431.4

(51) Int. Cl. C07K 14/715; A61K 38/00; A61K 47/64; A61P 9/00; A61P 11/00; A61P 13/12; A61P 17/00; A61P 27/02; A61P 35/00; G01N 33/68

(52) U.S. Cl. CPC C07K 14/7155; A61K 47/6425; A61P 9/00; A61P 11/00; A61P 13/12; A61P 17/00; A61P 27/02; A61P 35/00; G01N 33/6869; A61K 38/00; G01N 2333/7155

(57) ABSTRACT

An IL-11 binding receptor capable of binding to IL-11 and inhibiting IL-11 mediated signalling is disclosed. Also disclosed are compositions comprising the IL-11 binding receptor and methods using the IL-11 binding receptor.

### CROSS-REFERENCE TO RELATED APPLICATIONS

This application is a divisional of U.S. patent application Ser. No. 16/469,601, filed Jun. 13, 2019, which is a national stage filing under 35 U.S.C. 371 of International Patent Application No. PCT/EP2017/083032, filed Dec. 15, 2017, which claims the benefit of United Kingdom Patent Application No. 1621431.4, filed Dec. 16, 2016, each of which is herein incorporated by reference in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Feb. 25, 2022, is named E060170004US01-SEQ-GJM.txt and is 69,380 bytes in size.

### FIELD OF THE INVENTION

The present invention relates to decoy cytokine receptor molecules, specifically decoy IL-11 receptors capable of binding to interleukin 11 (IL-11).

### BACKGROUND OF THE INVENTION

Many fatal and incurable diseases are caused by organ failure due to excessive and maladaptive fibrosis (Rockey et al., 2015 Journal of Infectious Diseases 214, jiw176). Fibrotic disorders include both rare, genetically-driven diseases such as scleroderma, idiopathic pulmonary fibrosis and hypertrophic cardiomyopathy, dilated cardiomyopathy (DCM), and common diseases like atrial fibrillation, ventricular fibrillation, non-alcoholic fatty liver disease and diabetic kidney disease. Due to the significant impact on world-wide morbidity and mortality, there is a need to develop therapeutics to inhibit the fibrotic response (Nanthakumar et al., 2015 Nat Rev Drug Discov 14, 693-720).

A major hallmark of fibrosis is the pathologic activation of resident fibroblasts that drives their transition from a quiescent state to proliferating, secretory and contractile myofibroblasts (Hinz et al., 2010 Am J Pathology 170, 1807-1816). Stimuli such as mechanical stress and pro-fibrotic cytokines can activate fibroblasts. The TGF 1 pathway is considered to be of central importance for the fibrotic response (Leask and Abraham, 2004 The FASEB Journal 18, 816-827) and its inhibition is a therapeutic strategy that is under investigation (Gourdie et al., 2016 Nature Reviews Drug Discovery 15, 620-638). However, direct inhibition of multifunctional TGF1 is associated with severe side effects such as inflammation and cancer susceptibility.

### SUMMARY OF THE INVENTION

In one aspect, the present invention provides an IL-11 binding receptor, optionally isolated, which is capable of binding to IL-11 and inhibiting IL-11 mediated signalling.

In some embodiments, the IL-11 binding receptor is capable of inhibiting interaction between IL-11 and gp130. In some embodiments, the IL-11 binding receptor is capable of inhibiting interaction between IL-11 and IL-11 Rα.

In some embodiments, the IL-11 binding receptor comprises an amino acid sequence corresponding to the cytokine binding module of gp130. In some embodiments, the IL-11 binding receptor comprises an amino acid sequence corresponding to the cytokine binding module of IL-11 Rα.

In some embodiments, the IL-11 binding receptor comprises an amino acid sequence having at least 70% sequence identity to the cytokine binding module (CBM) of gp130. In some embodiments, the IL-11 binding receptor comprises an amino acid sequence having at least 70% sequence identity to the cytokine binding module (CBM) of IL-11 Rα.

In some embodiments, the IL-11 binding receptor comprises an amino acid sequence having at least 70% sequence identity to the sequence of SEQ ID NO: 19. In some embodiments, the IL-11 binding receptor comprises an amino acid sequence having at least 70% sequence identity to the sequence of SEQ ID NO: 20.

In some embodiments, the IL-11 binding receptor comprises:
(i) an amino acid sequence having at least 70% sequence identity to the sequence of SEQ ID NO: 19; and
(ii) an amino acid sequence having at least 70% sequence identity to the sequence of SEQ ID NO: 20.

In another aspect, the present invention provides an IL-11 binding receptor, optionally isolated, which is capable of binding to IL-11 and inhibiting IL-11 mediated signalling, comprising an amino acid sequence having at least 60% ace identity to the sequence of SEQ ID NO: 1 or 2.

In some embodiments in accordance with various aspects of the present invention, the IL-11 binding receptor is capable of inhibiting IL-11 trans signalling.

In another aspect, the present invention provides an IL-11 binding receptor, optionally isolated, which is capable of binding to IL-11, which is capable of inhibiting IL-11.

In some embodiments of the present invention the IL-11 binding receptor is conjugated to a drug moiety or a detectable moiety.

In another aspect, the present invention provides a complex, optionally in vitro and/or optionally isolated, comprising an IL-11 bind or invention bound to IL-11.

In another aspect, the present invention composing an IL-11 binding receptor according to the present invention, and at least one pharmaceutically-acceptable carrier.

In another aspect, the present invention provides an isolated nucleic acid encoding an IL-11 binding receptor according to the present invention.

In another aspect, the present inventor provides a vector comprising a nucleic acid according to the present invention.

In another aspect, the present invention provides a host cell comprising the vector according to the present invention.

In another aspect, the present invention provides a method for making an IL-11 binding receptor according to the present invention, comprising culturing the host cell according to the invention under conditions suitable for the expression of the IL-11 binding receptor, and recovering the IL-11 binding receptor.

In another aspect, the present invention provides an IL-11 binding position according to the present invention for use in therapy, or in a method of medical treatment.

In another aspect, the present invention provides an IL-11 binding receptor or composition according to the present invention for use in the treatment or prevention of fibrosis, or a disease/disorder characterised by fibrosis.

In another aspect, the present invention provides an IL-11 binding receptor or composition present invention for use in the treatment of a cancer.

In another aspect, the present invention provides the use of an IL-11 binding receptor or composition according to the present invention in the manufacture of a medicament for use in the treatment or prevention of fibrosis or a disease/disorder characterised by fibrosis.

In another aspect, the present invention provides the use of an IL-11 binding receptor or composition according to the present invention in the manufacture of a medicament for use in the treatment or prevention of a cancer.

In another aspect, the present invention provides a method of treating fibrosis comprising administering an IL-11 binding receptor or composition according to the present invention to a subject suffering from fibrosis or a disease/disorder characterised by fibrosis.

In another aspect, the present invention provides a method of treating cancer comprising administering an IL-11 binding receptor of composition according to the present invention to a subject suffering from a cancer.

In another aspect, the present invention provides an IL-11 binding receptor for use in method of treating a disease in which IL-11 mediated signalling is implicated in the pathology of the Base, in the IL-11 binding receptor is capable of inhibiting IL-11 trans signalling.

In another aspect, the present invention provides the use of an IL-11 binding receptor in the manufacture of a medicament for use in the treatment of a disease in which IL-11 mediated signalling is implicated in the pathology of the disease, wherein the IL-11 binding receptor is capable of inhibiting IL-11 trans signalling.

In another aspect, the present invention provides a method of treating a disease in which IL-11 mediated signalling is implicated in the pathology of the disease, comprising administering an IL-11 binding receptor to a subject suffering from the disease, wherein the IL-11 binding receptor is capable of inhibiting IL-11 trans signalling.

In another aspect, the present invention provides a method comprising contacting a sample, optionally in vitro, containing, or suspected to contain, IL-11 with an IL-11 binding receptor according to the present invention and detecting the formation of a complex of the IL-11 binding receptor with IL-11.

In another aspect, the present invention vides a method of diagnosing a disease or condition in a subject the method comprising contacting, in vitro sample from the subject with an IL-11 binding receptor according to the present invention ng e formation of a complex of the IL-11 binding receptor with IL-11.

In another aspect, the present invention provides a method of selecting or stratifying a subject for treatment with an IL-11-targeted agent, the method comprising contacting, in vitro, a sample from the subject with the IL-11 binding receptor according to the present invention and detecting the formation of a complex of the IL-11 binding receptor with IL-11.

In another aspect, the present invention provides the use of an IL-11 binding receptor according to the present invention for the detection of IL-11 in vitro or in vivo.

In another aspect, the present invention provides the use of an IL-11 binding receptor according to the present invention as an in vitro or in vivo diagnostic or prognostic agent.

### DESCRIPTION

The present invention relates receptor molecules with specificity for interleukin-11 (IL-11). The present disclosure describes the identification of IL-11/IL-11R signalling as a key mediator of fibrosis, and the generation and functional characterisation of recombinant IL-11 binding receptors. Therapeutic and diagnostic uses of the IL-11 binding receptors is also described.

### IL-11 and IL-11 Mediated Signaling

The decoy IL-11 receptors invention bind to interleukin 11. Interleukin 11 (IL-11), also known as adipogenesis inhibitory factor. Is a pleiotropic cytokine and a member of the IL-6 family of cytokines that includes IL-6, IL-11, IL-27, IL-31, oncostatin M (OSM), leukemia inhibitory factor (LIF), cardiotrophin-1 (CT-1), cardiotrophin like cytokine (CLC), ciliary neurotrophic factor (CNTF) and neuropoetin (NP-1).

IL-11 is transcribed with canonical signal peptide that ensures efficient secretion from cells. The immature form of human IL-11 is a 199 amino acid polypeptide whereas the mature form of IL-11 encodes a protein of 178 amino acid residues (Garbers and Scheller, Biol. Chem. 2013; 394 (9): 1145-1161). The human IL-11 amino acid sequence is available under UniProt accession no. P20609 (P20609.1 GI: 124294). Recombinant human IL-11 (oprelvekin) is also commercially available. IL-11 from other species, including mouse, rat pig, cow, several species of bony fish and primates, have also been cloned and sequenced.

In this specification “IL-11” refers to an IL-11 from any species and includes isoforms, fragments, variants or homologues of an IL-11 from any species. In some embodiments, the IL-11 is human IL-11, primate IL-11, non-human primate IL-11, rodent IL-1 murine IL-11, or mammalian IL-11. Similarly, in this specification “IL-11α” refers to an IL-11Rα from any species and includes isoforms, fragments, variants or homologues of an IL-11Rα from any species. In some embodiments, the IL-11Rα is human IL-11Rα, primate IL-11Rα, non-human primate IL-11Rα, rodent IL-11Rα, murine IL-11Rα, or mammalian IL-11Rα. Similarly, in this specification “gp130” refers to a gp130 from any species and includes isoforms, fragments, variants or homologues of gp130 from any species. In some embodiments, the gp130 is human gp130, primate gp130, non-human primate gp130, rodent gp130, murine gp130, or mammalian gp130.

IL-11 signals through a homodimer of the ubiquitously expressed β-receptor glycoprotein 130 (gp130; also known as glycoprotein 130. IL-6ST, IL-6-beta or CD130). Gp130 is a transmembrane protein that forms one subunit of the type I cytokine receptor with the IL-6 receptor family. Specificity is gained through an individual IL-11 α-receptor (IL-11Rα), which does not directly participate in signal transduction, although the initial cytokine binding event to the α-receptor leads to the final complex formation with the β-receptors. IL-11 activates a downstream signalling pathway, which is predominantly the mitogen-activated protein kinase (MAPK)-cascade and the Janus kinase/signal transducer and activator of transcription (Jak/STAT) pathway (Garbers and Scheller, supra).

Human gp130 (including the 22 amino acid signal peptide) is a 918 amino acid protein, and the mature form is 866 amino acids, comprising a 597 amino acid extracellular domain, a 22 amino acid transmembrane domain, and a 277 amino acid intracellular domain. The extracellular domain of the protein comprises the cytokine-binding module (CBM) of gp130. The CBM of gp130 comprises the Ig-like domain D1, and the fibronectin-type domains D2 and D3 of gp130. The amino acid sequence of human gp130 (Genbank accession no. NP_002175.2) and domains thereof are shown below:

Human gp130 (Genbank accession no. NP_002175.2):
(SEQ ID NO: 17)
MLTLQTWLVQALFIFLTTESTGELLDPCGYISPESPVVQLHSNFTAVCVLKEKCMDYFHVNANYIVWKTNHF
TIPKEQYTIINRTASSVTFTDIASLNIQLTCNILTFGQLEQNVYGITIISGLPPEKPKNLSCIVNEGKKMRCEWD
GGRETHLETNFTLKSEWATHKFADCKAKRDTPTSCTVDYSTVYFVNIEVWVEAENALGKVTSDHINFDPVY
KVKPNPPHNLSVINSEELSSILKLTWTNPSIKSVIILKYNIQYRTKDASTWSQIPPEDTASTRSSFTVQDLKPF
TEYVFRIRCMKEDGKGYWSDWSEEASGITYEDRPSKAPSFWYKIDPSHTQGYRTVQLVWKTLPPFEANGK
ILDYEVTLTRWKSHLQNYTVNATKLTVNLTNDRYLATLTVRNLVGKSDAAVLTIPACDFQATHPVMDLKAF
PKDNMLWVEWTTPRESVKKYILEWCVLSDKAPCITDWQQEDGTVHRTLYRGNLAESKCYLITVTPVYADG
PGSPESIKAYLKQAPPSKGPTVRTKKVGKNEAVLEWDQLPVDVQNGFIRNYTIFYRTIIGNETAVNVDSSHT

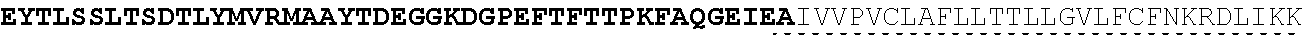

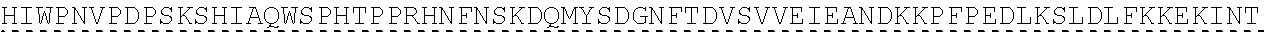

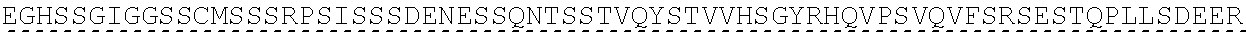

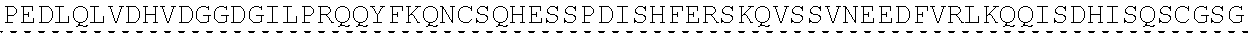

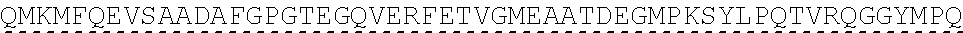

Signal peptide (residues 1-22); extracellular domain (residues 23-619);
D1 (residues 26-112); D2 (residues

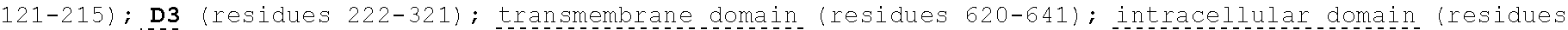

642-918).

An alignment of the ex acellular human gp130 and murine gp130 (UniProt: IL6RB_MOUSE) is shown in FIG. 20A (73.45% sequence identity), and an alignment of D1-D3 of human gp130 with the corresponding region of murine gp130 is shown in FIG. 20B (71.43% sequence identity).

Human IL-11Rα is a 422 amine acid polypeptide (Genbank accession no. NP_001136256.1 GI:218505839: UniProt Q14626) and shares ˜85% nucleotide and amino acid sequence identity with the murine IL-11Rα (Du and Williams, Blood Vol. 89, No. 11, Jun. 1, 1997). Two isoforms of IL-11Rα have been reported, which differ in the cytoplasmic domain (Du and Williams, supra). In some embodiments as used herein, the IL-11Rα may be IL-11Rα isoform 1 or IL-11Rα isoform 2.

Human IL-11Rα (including the 22 amino acid signal peptide) is a 422 amino acid protein, and the mature form is 400 amino acids, comprising 348 amino acid extracellular domain, a 20 amino acid transmembrane domain, and a 32 amino acid intracelluar domain. The extracellular domain of the protein comprises an Ig-like domain D1 and two fibronectin-type III domains D2 and D3. The cytokine binding module (CBM) of IL-11Rα comprises domains D2 and D3; D1 has been shown to be dispensable for binding of IL-11Rα to IL-11 (Schieinkofer et al. 2001, J. Mol. Biol. 306, 263-274; Pilanz et al., 1999; FEBS Lett. 450, 117-122). The amino acid sequence of human IL-11Rα (Genbank accession no. NP_001136256.1. UniProt Q14626) and domains thereof are shown below;

Human IL-11Ra (Genbank accession no. NP_001136256.1; UniProt Q14626):
(SEQ ID NO: 18)
MSSSCSGLSRVLVAVATALVSASSPCPQAWGPPGVQYGQPGRSVKLCCPGVTAGDPVSWFRDGEPKLLQ

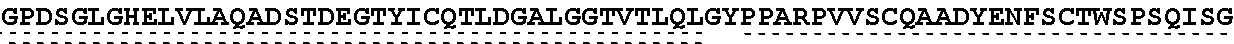

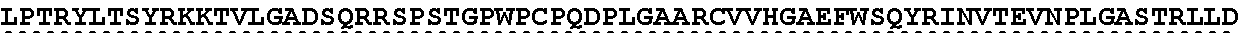

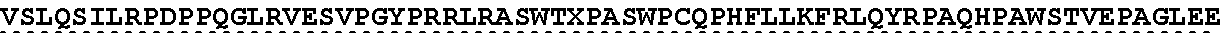

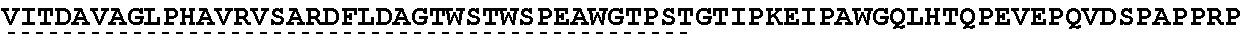

SLQPHPRLLDHRDSVEQVAVLASLGILSFLGLVAGALALGLWLRLRRGGKDGSPKPGFLASVIPVDRRPGAP
NL
Signal pepide (residues 1-22); extracellular domain (residues 23-619);
D1 (residues 26-112); D2 (residues

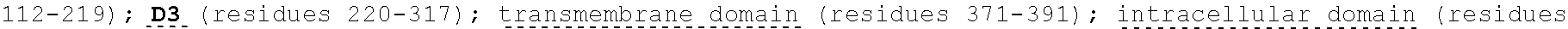

642-918).

An alignment of the extracellular domains of human IL-11Rα and murine IL-11Rα (UniProt: 111RA_MOUSE) is shown in FIG. 21A (85.06% sequence identity), and an alignment of D2-D3 of human IL-11Rα with the corresponding region of murine IL-11Rα is shown in FIG. 21B (91.26% sequence identity).

The IL-11 receptor α-chain (IL-11Rα) shares many structural and functional similarities with the IL-6 receptor α-chain (IL-6Rα). The extracellular domain shows 24% amino acid identity including the characteristic conserved Trp-Ser-X-Trp-Ser (WSXWS) motif. The short cytoplasmic domain lacks the Box 1 and 2 regions that are required for activation of the JAK/STAT signalling pathway.

IL-11Rα binds is ligand a low affinity (Kd˜10 nmol/L) and alone is insufficient to transduce a biological signal. The generation of a high affinity receptor (Kd˜400 to 800 pmol/L) capable of signal transduction requires co-expression of the IL-11Rα and gp130 (Curtis et al (Blood 1997 Dec. 1; 90 (11): 4403-12; Hilton et al., EMBO J 13:4765, 1934; Nandurkar of al., Oncogene 12:585, 1996). Binding of IL-11 to cell-surface IL-11Rα induces heterodimerization, tyrosine phosphorylation, activation of gp130 and MAPK and/or Jak/STAT signalling as described above.

The receptor binding sites on murine IL-11 have been mapped and three sites—sites I, II and III—identified Binding to gp130 is reduced by substitutions in the site II region and by substitutions in the site III region. Site III mutants show no detectable agonist activity and have IL-11Rα antagonist activity (Cytokine Inhibitors Chapter 8: edited by Gennaro Ciliberto and Rocco Savino, Marcel Dekker, Inc. 2001).

In principle, a soluble IL-11Rα can also form e soluble complexes with IL-11 (Pflanz et al., 1999 FEBS Let, 450, 117-122) raising the possibility that, similar to IL-6, IL-11 may in some instances bind soluble IL-11Rα prior to binding cell-surface gp130 (Garbers and Scheller, supra). Curtis et al (Blood 1997 Dec. 1; 90 (11): 4403-12) describe expression of a soluble murine IL-11 receptor alpha chain (sIL-11Rα) and examined signalling cells expressing gp130. In the presence of gp130 but not transmembrane IL-11R the sIL-11R mediated IL-11 dependent differentiation of M1 leukemic calls and proliferation in Ba/F3 cells and early intracellular events including phosphorylation of gp130, STAT3 and SHP2 similar to signalling through transmembrane IL-11R.

As used herein, IL-11/IL-11R signalling refers to signalling mediated by IL-11 and/or IL-11Ra, fragments of IL-11 and/or IL-11Rα and polypeptide complexes comprising IL-11, IL-11Rα and/or fragments thereof. IL-11/IL-11R signalling involves binding of IL-11 and/or IL-11Rα to gp130, and consequent activation of signalling through gp130.

Activation of signalling through cell-membrane bound gp130 by IL-11 bound to soluble IL-11Rα has recently been demonstrated (Lokau et al., 2016 Cell Reports 14, 1761-1773). This so-called IL-11 trans signalling may be a very important component of IL-11/IL-11R signalling, and may even be the most common form of IL-11/IL-11R signalling, because whilst the expression of IL-11Rα is restricted to a relatively small subset of cell types, gp130 is expressed on a wide range of cell types.

As used herein, ‘IL-11 trans signalling’ is used to refer to signalling which is triggered by binding of IL-11 bound to IL-11Rα, to gp130. The IL-11 may be bound to IL-11Rα as a non-covalent complex. The gp130 is membrane-bound and expressed by the cell in which signalling occurs following binding of the IL-11:IL-11Rα complex to gp130. In some embodiments the IL-11Rα may be a soluble IL-11Rα. In some embodiments, the soluble IL-11Rα is a soluble (secreted) isoform of IL-11Rα (e.g. lacking a transmembrane domain). In some embodiments, the soluble IL-11Rα is the liberated product of proteolytic cleavage of the extracellular domain of cell membrane bound IL-11Rα. In so the embodiments, the IL-11Rα may be cell membrane-bound, and signalling through gp130 may be triggered by binding of IL-11 bound to cell-membrane bound IL-11Rα.

In this specification an IL-11 receptor (IL-11R) refers to a polypeptide capable of binding IL-11 and inducing signal transduction in cells expressing gp130. An IL-11 receptor may be from any species and includes isoforms, fragments, variants or homologues of an IL-11 receptor from any species. In preferred embodiments the species is human (Homo sapiens). In some embodiments the IL-11 receptor may be IL-11Rα. Isoforms, fragments, variants of homologues of an IL-11Rα may optionally of characterised as having at least 70%, preferably one of 80%, 85%, 90%, 91%, 92%, 93%, 94%, 95%, 98%, 97%, 98%, 99% or 100% amino acid sequence identity to the amino acid sequence of IL-11Ra from a given species, e.g. human, isoforms, fragments, variants or homologues of an IL-11Rα may optionally be characterised by ability to bind IL-11 (preferably from the same species) and stimulate signal transduction in cells expressing IL-11Rα and gp130 (e.g. as described in Curtis e at Blood, 1997, 90(11) or Karpovich et al. Mol. Hum. Reprod. 2003 9(2): 75-80). A fragment of an IL-11 receptor may be of any length (by number of amino acids), although may optionally be at least 25% of the length of the mature IL-11Rα and have a maximum length of one of 50%, 75%, 80%, 85%, 90%, 91%, 92%, 93%, 94%, 95%, 96%, 97%, 98%, or 99% of the length of the mature IL-11Rα. A fragment of an IL-11 receptor fragment may have a minimum length of 10 amino acids, and a maximum length of ones of 15, 20, 25, 30, 40, 50, 100, 110, 120, 130, 140, 150, 160, 170, 180, 190, 200, 250, 300, 400, or 415 amino acids.

In some embodiments, the IL-11Rα may comprise, or consist of, the extracellular domain of IL-11Rα, which corresponds to amino acids 24 to 370 of the amino acid sequence of UniProt Q14626. In some embodiments, the IL-11Rα may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of the extracellular domain of IL-11Rα from a given species.

In some embodiments, the IL-11 is mammalian IL-11 (e.g. cynomolgous, human and/or rodent (e.g. rat and/or murine) IL-11). Isoforms, fragments, variants or homologues of an IL-11 may optionally be characterised as having at least 70%, preferably one of 80%, 85%, 90%, 91%, 92%, 93%, 94%, 95%, 98%, 97%, 98%, 99% or 100% amino acid sequence identity to the amino acid sequence of immature or mature IL-11 from a given species, e.g. human. Isoforms, fragments, variants or homologues of an IL-11 may optionally be characterised by ability to bind IL-11Rα (preferably from the same species) and stimulate signal transduction in cells expressing IL-11Rα and gp130 (e.g. a described in Curtis at al. Blood, 1997, 90(11); or Karpovich at al Mol. Hum. Reprod. 2003 9(2) 75-80). A fragment of IL-11 may be of any length (by number of amino acids), although may optionally be at least 25% of the length of mature IL-11 and may have a maximum length of ore of 50%, 75%, 80%, 85%, 90%, 91%, 92%, 93%, 94%, 95%, 96%, 97%, 98%, or 99% of the length of mature IL-11. A fragment of IL-11 may have a minimum length of 10 amino acids, and a maximum length of one of 15, 20, 25, 30, 40, 50, 100, 110, 120, 130, 140, 150, 160, 170, 180, 190 or 195 amino acids

IL-11 has been proposed to function mainly as a thrombopoietin growth factor, which underpinned the use of recombinant IL-11 (Neumega (Oprelvekin)) as a therapeutic agent to increase platelet count. TGFβ1 has been shown to induce IL-11 expression in fibroblasts (Elias at al., 1994 J. Immunol. 152, 2421-2429).

The role of IL-11/IL-11R signalling in fibrosis is not clear. The majority of studies suggest anti-fibrotic function for IL-11/IL-11R signalling in the heart (Obans at al., 2010 Circulation 121, 684-491; Obana et al., 2012 Heart and Circulatory Physiology 303, H569-77) and kidney (Ham et al, 2013 Anesthesiology 119, 1389-1401; Stangou at al. 2011 J. Nephrol 24, 106-111). Kurahara et al. J. Smooth Muscle Res. 2016; 52; 78-92 describes IL-11 as an anti-fibrotic cytokine, and suggests that IL-11/IL-11R signalling suppresses αSMA expression.

IL-11 has also been suggested to be an anti-inflammatory factor in several tissues and chronic Inflammatory diseases (Trepicchio and Dorner, 1998 Expert Opin Investig Drugs 7, 1501-1504; Zhu at al., 2015 PLoS ONE 10, e0126296). Those studies suggest that the observed secretion of IL-11 in response to TGFβ1 is a protective mechanism.

On the other hand, it has been suggested that IL-11/IL-11R signalling may be Involved in pathology of diseases of the lung. Inhibition of IL-11/IL-11R signalling either via antibodies or a mutated recombinant IL-11 in a model of tuberculosis revealed a positive feedback loop in vivo and diminished histopathology of the lung (Kapina et al., 2011 PLoS ONE 6, e21878; Shepelkova et al., 2018 Journal of Infectious Diseases 214, jw176), fibrosis of the murine airway has been associated with IL-11 expression (Tang at al., 1996 The Journal of Clinical Investigation 98, 2845-2853). When the pro-fibrotic function of IL-13 in lung tissue was investigated in IL-11RA−/− mice, IL-11/IL-11R signalling was implicated in the mechanism (Cher et at, 2005 J. Immunol. 174, 2305-2313).

IL-11 was also found to be elevated in the airway of patients with severe asthma (Minshall et al., 2000 Respiratory Research 14, 1-14), is overexpressed in the lungs of IPF patients (Lindahl at al., 2013 Respiratory Research 14, 1-14) and is elevated in skin lesions in atopic dermatitis patients (Toda at al., 2003 J Allergy Clin Immun 111, 875-881). It is uncertain whether these associations are due to increased IL-11 gene/protein expression as a response to disease processes, or whether IL-11 is an effector of disease processes.

### IL-11 Binding Receptors

The present invention provides an IL-11 binding receptor. An IL-11 binding receptor is a peptide/polypeptide or plurality (e.g. non-covalent complex) thereof which is capable of binding to IL-11. By ‘receptor’ we Include fragments and derivatives thereof. As used herein, an IL-11 binding receptor is a peptide/polypeptide or plurality thereof capable of binding to IL-11 in the manner of a cytokine receptor biding to its ligand. IL-11 binding receptors according to the present Invention may be provided in isolated form.

An IL-11 binding receptor is not an antibody or an antigen-binding fragment of an antibody which is specific for IL-11. An IL-11 binding receptor lacks sequences encoding heavy and light chain complementarity determining regions (CDRs) and/or heavy chain and light chain variable regions of an antibody or antigen binding fragment capable of specific binding to IL-11.

As used herein, a “peptide” is a chain of two or more amino acid monomers linked by peptide bonds. A peptide typically has a length in the region of about 2 to 50 amino acids. A ‘polypeptide’ is a polymer chain of two or more peptides. Polypeptides typically have a length greater than about 50 amino acids.

IL-11 binding receptors according to the present invention bind to IL-11 (interleukin 11) They are soluble (not membrane bound) receptor molecules. In some embodiments, the IL-11 binding receptor binds to human IL-11. In some embodiments, the IL-11 binding receptor binds to non-human primate IL-11. In some embodiments, the IL-11 binding receptor binds to murine IL-11.

In some embodiments, the IL-11 binding receptors bind to IL-11-containing molecules/complexes (e.g. IL-11:IL-1Rα complex).

IL-11 binding receptors according to the present invention preferably bind and IL-11 and IL-11 containing complexes, and thereby make these species unavailable for binding to gp130, IL-11Rα and/or gp130:IL-11Rα receptors. As such, the present IL-II binding receptors act as ‘decoy’ receptor for IL-11 and IL-11 containing complexes, much in the same way that etanercept acts as a decoy receptor for TNFα. IL-11 mediated signalling is reduced as compared to the level of signalling in the absence of the IL-11 binding receptors according to the invention.

The IL-11 binding receptors according to the present invention preferably bind to IL-11 through one or more cytokine binding modules (CBM). The CBMs are, or are derived from or homologous to, the CBMs of naturally occurring receptor molecules for IL-11. For example, the IL-11 binding receptors of the present invention may comprise, or consist of, re or more CBMs which are from, are derived from or homologous to the CBM of gp130 and/or IL-11Rα.

As explained herein, IL-11 and complexes containing IL-11 (i.e. IL-11 in complex with IL-11Rα) are capable of activating IL-11 mediated signalling through binding to cell membrane bound gp130. The IL-11 binding receptors of the present invention are able to bind to the IL-11 and IL-11 containing species such as to inhibit the ability to interact with cell membrane bound gp130, and thereby inhibit IL-11 mediated signalling.

The IL-11 binding receptors of the present invention are not agonists of IL-11 mediated signalling. That is, the IL-11 binding receptors do not promote IL-11 mediated signalling. The IL-11 binding receptors do not bind to IL-11 to form IL-11 containing complexes capable of binding to cell membrane bound receptor for IL-11 to initiate IL-11 mediated signalling.

This is preferably achieved through binding of the IL-11 binding receptor to the region of IL-11 which is required for binding to cell membrane bound gp130. That is, IL-11 binding receptors capable of binding to IL-11 in the region bound by gp130 reduce the amount of IL-11 and IL-11 containing species available to bind to and activate signalling through cell membrane bound receptors for IL-11 (e.g. gp130 and IL-11Rα:gp130 receptor complexes).

Accordingly, in some embodiments, the IL-11 binding receptor Is capable of binding to IL-11 in the region of IL-11 which s bound by gp130. In some embodiments, the IL-11 binding receptor is capable of binding to IL-11 In the same region of IL-11, or an overlapping region of IL-11, as the region of IL-11 which is bound by gp130. Ability to bind to IL-11 in the same region or an overlapping region of as the region bound by gp130 can be analysed using a competitive binding assay, such as a competition ELISA. In such assay, observation of a reduction/decrease in the level of interaction between IL-11 or IL-11:IL-11Rα and e.g. gp130, or IL-11Rα:gp130 in the presence of—or following incubation of one or both of the interaction partners with—the IL-11 binding receptor, as compared to the level of interaction in the absence of the IL-11 binding receptor indicates that the IL-11 binding receptor binds to the same region or overlapping region of IL-11 as the region bound by gp130. Whether an IL-11 binding receptor according to the present invention binds to IL-11 in the same or same region or overlapping region of IL-11 as the region bound by gp130 can also be determined by analysis of interaction using various methods well known in the art, including X-ray co-crystallography analysis of receptor-ligand complexes, peptide scanning, mutagenesis mapping, hydrogen-deuterium exchange analysis by mass spectrometry, phage display and proteolysis-based ‘protection’ methods. Such methods are described, for example, in Gershoni et al., BioDrugs, 2007, 21(3):145-156, which is hereby incorporated by reference in its entirety.

In some embodiments, the IL-11 binding receptor comprises, or consists of, an amino acid sequence corresponding to the cytokine binding module of gp130. Herein “an amino acid sequence corresponding to the cytokine binding module of gp130” may be the amino acid sequence of the cytokine binding module of gp130, or an amino acid sequence which is capable of binding to IL-11 and having at least 60%, e.g. one of at least 65%, 70%, 75%, 80%, 85%, 90%, 91%, 92%, 93%, 94% 95%, 96%, 97%, 98% or 99% sequence identity to the amino acid sequence of the cytokine binding module of gp130. The gp130 and IL-11 may be from any species, and includes isoforms, fragments, variants or homologues from any species.

In some embodiments, the IL-11 binding receptor comprises, or consists of, the cytokine binding module of gp130. In some embodiments, the IL-11 binding receptor comprises, or consists of, n amino acid sequence having at least 70% sequence identity to the cytokine binding module (CBM) of gp130.

The cytokine binding module of human gp130 corresponds to positions 26 to 321 of human gp130 (Genbank accession no, NP_002175.2) shown in SEQ ID NO:17, that is:

(SEQ ID NO: 19)
DPCGYISPESPVVQLHSNFTAVCVLKEKCMDYFHVNANYIVWKTNHFTIPKEQYTIINRTASSVTFTDIASLNI

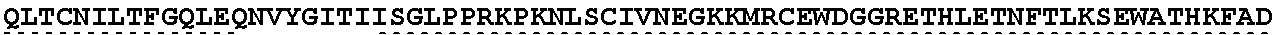

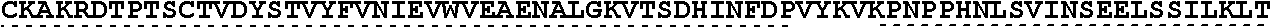

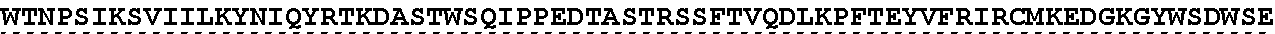

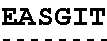

D1 (residues 26-112 of SEQ ID NO: 17); D2 (residues 121-215 of SEQ ID NO: 17); D3 (residues 222-321 of
SEQ ID NO: 17).

The skilled person is well able to Identify cytokine binding module for a given homologue of human gp130 by routine methods. For example, FIG. 20B shows an alignment of the cytokine binding module of human gp130 with the homologous mouse protein.

In some embodiments the IL-11 binding receptor comprises or consists of, an amino acid sequence having at least 60%, 65%, 70%, 75%, 80%, 85%, 90%, 91%, 92%, 93%, 94%, 95%. 98%, 97%; 98% or 99% sequence identity to the cytokine binding module (CBM) of gp130. In some embodiments the IL-11 binding receptor comprises, or consists of, an amino acid sequence having greater then 70%, 75%, 80%, 85%, 90%, 91%, 92%, 93%, 94%, 95%, 96%, 97%. 96% or 99% sequence identity to the cytokine binding module (CBM) of gp130.

Sequence identity between amino acid sequences can be determined by methods known to the person skilled in the art. For example, to determine the percent identity of two amino acid sequences, the sequences to be compared an be aligned (e.g., gaps can be introduced m one or both of a first and a second amino acid sequence for optimal alignment), and the amino acids at corresponding positions can be compared. When a position in the first sequence is occupied by the same amino acid as the corresponding position in the second sequence, the sequences have ‘identity’ at that position. The percent identity between two sequences is a function of the number of identical positions, taking into account the number of gaps, and the length of each gap, which need to be introduced for optimal alignment of the sequences. Sequence identity is preferably determined over the full length of the amino acid sequences being compared.

The IL-11 binding receptor according to the present invention may be capable of binding to IL-11 in the region of IL-11 bound by IL-11Rα.

In some embodiments, the IL-11 binding receptor is capable of binding to IL-11 in the same region of IL-11, or an overlapping region of IL-11, as the region of IL-11 which is bound by IL-11Rα. Ability to bind to IL-11 in the same region or an overlapping region of as the region bound by IL-11Rα can be analysed using a competitive binding assay, such as a competition ELISA. In such assay, observation of a reduction/decrease in the level of interaction between IL-11 and e.g. IL-11Rα or IL-11Rα:gp130 in the presence of—or following incubation of one or both of the interaction partners with—the IL-11 binding receptor, as compared to the level of interaction in the absence of the IL-11 binding receptor indicates that the IL-11 binding receptor binds to the same region or overlapping region of IL-11 as the region bound by IL-11Rα. Whether an IL-11 binding receptor according to the present invention binds to IL-11 in the same or same region or overlapping region of IL-11 as the region bound by IL-11Rα can also be determined e.g. by X-ray co-crystalography analysis of receptor-ligand complexes, peptide scanning, mutagenesis mapping, hydrogen-deuterium exchange analysis by mass spectrometry, phage display and proteolysis-based ‘protection’ methods (described, for example, in Gershoni et al. BioDrugs, 2007, 21(3):145-156, incorporated by reference hereinabove).

In some embodiments, the IL-11 binding receptor comprises an amino acid sequence corresponding to the cytokine binding module of IL-11Rα. Herein, “an amino acid sequence corresponding to the cytokine binding module of IL-11Rα” may be the amino acid sequence of the cytokine binding module of IL-11Rα, or an amino acid sequence which is capable of binding to IL-11 and having at let 60%, e.g. one of at least 65%, 70%, 75%, 80%, 85%, 90%, 91%, 92%, 93%, 94%, 95%, 96%, 97%, 98% or 99% sequence identity to the amino acid sequence of the cytokine binding module of IL-11Rα. The IL-11Rα and IL-11 may be from any species, and includes isoforms, fragments, variants or homologues from any species.

In some embodiments, the IL-11 binding receptor comprises the cytokine binding module of IL-11Rα. In some embodiments, the IL-11 binding receptor comprises an amino acid sequence having at least 70% sequence identity to the cytokine binding module (CBM) of IL-11Rα.

The cytokine binding module of human IL-11 Ra corresponds to positions 112 to 317 of human IL-11Rα (Genbank accession no. NP_001136256.1; UNiProt Q14626) shown in SEO ID NO:18, that is:

(SEQ ID NO: 20)

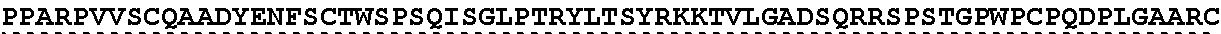

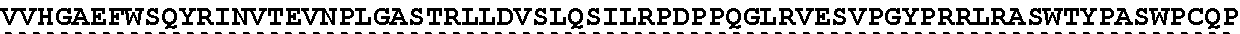

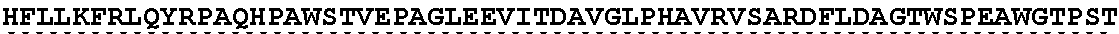

D2 (residues 112-219 of SEQ ID NO: 18); D3 (residues 220-317 of SEQ ID NO: 18).

The skilled person is well able to identify cytokine binding module for a given homologue of human IL-11Rα by routine methods. For example, FIG. 21B shows an alignment of the cytokine binding module of human IL-11Rα with the homologous mouse protein.

In some embodiments the IL-11 binding receptor comprises an amino acid sequence having at least 60%, 65%, 70%, 75%, 80%, 85%, 90%, 91%, 92%, 93%, 94%, 95%, 96%, 97%, 98% or 99% sequence identity to the cytokine binding module (CBM) of IL-11Rα. In same embodiments the IL-11 binding receptor comprises an amino acid sequence having greater than 70%, 75%, 80%. 8%. 90%, 91%, 92%, 93%, 94%, 95%, 96%, 97%, 98% or 99% sequence identity to the cytokine binding module (cBM) of IL-11Rα.

The IL-11 binding receptor according to the present invention may be capable of binding to IL-11 in the region of IL-11 bound by gp130, and may also be capable of binding to IL-11 in the region of IL-11 bound by IL-11Rα.

IL-11 binding receptor molecules capable of such binding interact with IL-11 with high affinity, to form stable complexes. IL-11 binding receptors capable of binding to IL-11 in the regions bound by both gp130 and IL-11Rα reduce the amount of IL-11 and IL-11 containing species available to bind to end activate signalling through cell membrane bound receptors for IL-11 (e.g. gp130 and IL-11Rα:gp130 receptor complexes).

In some embodiments, the IL-11 binding receptor is capable of binding to IL-11 In the same region of IL-11, or an overlapping region of IL-11, as the region of IL-11 which is bound by gp130; and is capable of binding to IL-11 in the same region of IL-11, or an overlapping region of IL-11, as the region of IL-11 which is bound by IL-11Rα.

In some embodiments, the IL-11 binding receptor comprises, or consists of, an amino acid sequence corresponding to the cytokine binding module of gp130 and an amino acid sequence corresponding to the cytokine binding module of IL-11Rα.

In some embodiments, the IL-11 binding receptor comprises, or consists of, the cytokine binding module of gp130 and the cytokine binding module of IL-11Rα. In some embodiments, the IL-11 binding receptor comprises, or consists of, an amino acid sequence having at least 70% sequence identity to the cytokine binding module (CBM) of gp130 and an amino acid sequence having at least 70% sequence identity to the cytokine binding module (CBM) of IL-11Rα.

In some embodiments the IL-11 binding receptor comprises or consists of: an amino acid sequence having at least 60%, 65%, 70%, 75%, 80%, 85%, 90%, 91%, 92%, 93%, 94%, 95%, 96%, 97%, 98% or 99% sequence Identity to the cytokine binding module (CBM) of gp3; and an amino acid sequence having at least 60%, 65%, 70%, 75%, 80%, 85%, 90%, 91%, 92%, 93%, 94%, 95%, 96%, 97%, 98% or 99% sequence identity to the cytokine binding module (CSM) of IL-11Rα. In some embodiments the IL-11 binding receptor comprises or consists of: an amino acid sequence having greater then 70%, 75%, 80%, 85%, 90%, 91%, 92%, 93%, 94%, 96%, 96%, 97%, 98% or 99% sequence identity to the cytokine binding module (CSM) of gp130; and an amino acid sequence having greater than 70%, 75%, 80%, 85%, 90%, 91%, 92%, 93%, 94%, 95%, 96%, 97%, 98% or 99% sequence identity to the cytokine binding module (CBM) of IL-11Rα.

In some embodiments, the IL-11 binding receptor comprises, or consists of, an amino acid sequence having at least 70% sequence identity to a cytokine binding module of a decoy IL-11 receptor as described herein, e.g. decoy IL-11 receptor 1 (D11R1) or decoy IL-11 receptor 2 (D11R2). The amino acid sequences for D11R1 and D11R2 are shown in FIGS. 17A and 17B, respectively.

In some embodiments, the cytokine binding module (CBM) of gp130 comprises or consists of an amino acid sequence as shown in SEQ ID NO:19 or SEQ ID NO:4.

In some embodiments the IL-11 binding receptor comprises, or consists of, an amino acid sequence having at least 60%, 65%, 70%, 75%, 80%, 85%, 90%, 91%, 92%, 93%, 94%, 95%, 96%, 97%, 98% or 99% sequence identity to SEQ ID NO: 19 or 4. In some embodiments the IL-11 binding receptor comprises, or consists of, an amino acid sequence having greater than 70%, 75%, 60%, 85%, 90%, 91%, 92%, 93%, 94%, 95%, 96%, 97%, 98% or 99% sequence identity to SEQ ID NO:19 or 4.

In some embodiments, the cytokine binding module (CBM) of IL-11Rα comprises or consists of an amino acid sequence as shown in SEQ ID NO:20 or SEO ID NO:6.

In some embodiments the IL-11 binding receptor comprises an amino acid sequence having at least 60%, 65%, 70%, 75%, 80%, 85%, 90%, 91%, 92%, 93%, 94%, 95% 96%, 97%, 98% or 99% sequence identity to SEQ ID NO:20 or 8. In some embodiments the IL-11 binding receptor comprises an amino acid sequence having greater than 70%, 75%, 80%, 85%, 90%, 91%, 92%, 93%, 94%, 95%, 96%, 97% 98% or 99% sequence identity to SEQ ID NO:20 or 6.

In some embodiments the IL-11 binding receptor comprises, or consists of, an amino acid sequence having at least 60%, 65%, 70%, 75%, 80%, 85%, 90%, 91%, 92%, 93%, 94%, 95%, 96%, 97%, 98% or 99% sequence identity to SEQ ID NO:19 or 4 and an amino acid sequence having at least 60%, 85%, 70%, 76%, 80%, 85%, 90%, 91%, 92%, 93%, 94%, 95%, 96%, 97%, 98% or 99% sequence identity to SEQ ID NO:20 or 6 In some embodiments the IL-11 binding receptor comprises, or consists of, an amino acid sequence having greater than 70%, 75%, 80%, 85%, 90%, 91%, 92%, 93%, 94%, 95%, 96%, 97%, 98% or 99% sequence Identity to SEQ ID NO: 19 or 4 end an amino acid sequence having greater than 70%, 75%, 80%, 85%, 90%, 91%, 92%, 93%, 94%, 95%, 96%, 97%, 98% or 99% sequence identity to SEQ ID NO:20 or 6.

In some embodiments, the IL-11 binding receptor comprises or consists essentially of:
(i) a region corresponding to the cytokine binding module of gp130; and
(ii) a region corresponding to the cytokine binding module of IL-11Rα.

In some embodiments, the IL-11 binding receptor is a polypeptide comprising or consisting essentially of a region corresponding to the cytokine binding module of gp130, and a region corresponding to the cytokine binding module of IL-1Rα.

The IL-11 binding receptor may be a single polypeptide or fusion protein comprising, or consisting of, a region corresponding to the cytokine binding module of gp130 (as described herein), optionally a linker, and a region corresponding to the cytokine binding module of IL-11Rα (as described herein).

In some embodiments, the IL-11 binding receptor comprises an amino acid sequence having at least 70% sequence identity to the cytokine binding module (CBM) of gp130 and an amino acid sequence having at least 70% sequence identity to the cytokine binding module (COM) of IL-11Rα In a single polypeptide.

In some embodiments the IL-11 binding receptor comprises or consists of:
(i) an amino acid sequence having at least 60%, 65%, 70%, 75%, 80%, 85%, 90%, 91%, 92%, 93%, 94%, 95%, 96%, 97%, 98% or 99% sequence identity to SEQ ID NO:19 or 4, or the cytokine binding module (CBM) of gp130; and
(ii) an amino acid sequence having at least 60%, 65%, 70%, 75%, 80%, 85%, 90%, 91%, 92%, 93%, 94%, 95%, 96%, 97%, 98% or 99% sequence identity to SEQ ID NO:20 or 6, or the cytokine binding module (CBM) of IL-11Rα.

In some embodiments the IL-11 binding receptor comprises, or consists of:
(i) In se embodiments the IL-11 binding receptor comprises an amino acid sequence having greater than 70%, 75%, 80%, 85%, 90%, 91%, 92%, 93%, 94%, 95%, 96% 97%, 98% or 99% sequence identity to SEQ ID NO:19 or 4, or the cytokine binding module (CBM) of gp130; and
(ii) an amino acid sequence having greater than 70%, 75%, 80%, 85%, 90%, 91%, 92%, 93%, 94%, 95%, 96%, 97%, 98% or 99% sequence identity to SEQ ID NO:20 or 6, or the cytokine binding module (CBM) of IL-11Rα.

In some embodiments, the IL-11 binding receptor comprises a linker between the IL-11 binding sequences. The linker may comprise or consist of an amino acid sequence, and may be covalently bonded (e.g. by peptide bonds) to ends of the sequences according to (i) and (i). As such, the IL-11 binding receptor may comprise, or consist of, a contiguous amino acid sequence having an amino acid sequence encoding the cytokine binding module of gp130, an amino acid sequence encoding a linker and an amino acid sequence encoding the cytokine binding module of IL-11Rα, where the amino acid sequence encoding the linker is positioned between the amino acid sequences encoding the respective cytokine binding domains.

The linker may be a peptide or polypeptide linker. The linker may be a flexible linker. Amino acid sequences of flexible linkers are known to the skilled person, and are described, for example in Chen at al., Adv Drug Deliv Rev (2013) 65(10): 1357-1389, which is hereby incorporated by reference in its entirety. In some embodiments the flexible linker sequence comprises serine and glycine residues.

The linker may be any length and form as long as it does not substantially constrain the amino acid sequences of (i) and/or (ii) so as to inhibit their ability to interact with IL-11 In some embodiments the linker is a peptide/polypeptide consisting of an amino acid sequence of 1-500, 1-400, 1-300, 1-200, 1-100, 5-75, or 10-60 amino acid residues.

In some embodiments, the linker sequence of IL-11 binding receptor according to the present invention comprises, or consists of an amino acid sequence having at least 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:5 or 8.

In some embodiments, the IL-11 binding receptor according to the present invention comprises a leader sequence (also known as a signal peptide or signal sequence). Leader sequences normally consist of a sequence of 530 hydrophobic amino adds, which form a single alpha helix Secreted proteins and proteins expressed at the cell surface often comprise leader sequences. The leader sequence may be present at the N-terminus of the IL-11 binding receptor, and may be present in the newly synthesized receptor. The leader sequence provides for efficient intracellular trafficking and secretion of the IL-11 binding receptor. Leader sequences are often removed by cleavage, and thus are not comprised in the mature IL-11 binding receptor.

Leader sequences are known for many proteins, and are recorded in databases such as GenBank, UniProt. Swiss-Prot, TrEMBL Protein Information Resource, Protein Data Bank, Ensembl, and InterPro, and/or can be identified/predicted e.g. using amino ac sequence analysis tools such as SignalP (Petersen et at, 2011 Nature Methods 8: 785-786) or Signal-BLAST (Frank and Sippi, 2008 Bioinformatics 24: 2172-2176).

In some embodiments, the leader sequence of the IL-11 binding receptor of the present invention comprises, or consists of, an amino acid sequence having at least 80%, 85%, 86%, 87%, 88%, 89%, 90%, 91%, 92%, 93%, 94%, 95%, 98%, 97%, 98%, 99%, or 100% sequence identify to the amino acid sequence of SEQ ID NO:3.

In some embodiments, the IL-11 binding receptor according to the present invention may be provided with a relative arrangement of amino acid sequences encoding a gp130 cytokine binding module: an IL-11Rα cytokine binding module and a linker according to one of the following:
N term-[signal sequence]-[gp130 CBM]-[linker]-[IL-11Rα CBM]-C term;
N term-[gp130 CBM]-[linker]-[IL-11Rα-CBM]-C term;
N term-[signal sequence]-[IL-11Rα CBM]-[linker]-[gp130 CBM]-C term; or
N term-[IL-11Rα CBM]-[linker]-[gp130 CBM]-C term.

In some embodiments, the IL-11 binding receptor according to the present invention may comprise a linker having an amino acid sequence comprising or consisting of the amino acid sequence of SEQ ID NO:5 or 8.

IL-11 binding receptors wording to the present invention are distinct from naturally-occurring IL-11 binding molecules, e.g. a naturally occurring receptor for IL-11 or IL-11 containing complex (e.g. IL-11:IL-11Rα).

For example, in some embodiments, the IL-11 binding receptor comprises amino acid sequence corresponding only to the cytokine binding module of gp130, and not other regions of gp130. The IL-11 binding receptor of the present invention preferably kicks amino acid sequence corresponding to regions of gp130 other than the cytokine binding module of gp130 (i.e. D1 to D3) In some embodiments, the IL-11 binding receptor lacks amino acid sequence corresponding to the intracellular domain of gp130 and/or the transmembrane domain of gp130. In some embodiments the IL-11 binding receptor lacks amino acid sequence corresponding to the stalk region of gp130.

In some embodiments, the IL-11 binding receptor of the present invention preferably lacks amino acid sequence corresponding to regions of gp130 other than positions 23 to 321 of SEQ ID NO:17. In some embodiments the IL-11 binding receptor lacks amino acid sequence corresponding to positions 322 to 619 of SEQ ID NO:17.

Herein, an amino acid sequence which corresponds' to a reference region or sequence of a given peptide/polypeptide has at least 60% e.g. one of at least 65%, 70%, 75%, 80%, 85%, 90%, 91%, 92%, 93%, 94%, 95%, 96%, 97%, 98% or 99% sequence identity to the amino acid sequence of the reference region/sequence.

Similarly, in some embodiments, the IL-11 binding receptor comprises amino acid sequence corresponding only to the cytokine binding module of IL-11Rα, and not other regions of IL-11Rα. That is, the IL-11 binding receptor of the present invention preferably lacks amino add sequence corresponding to regions of IL-11Rα other than the cytokine binding module of IL-11Rα (i.e. D2 to D3). In some embodiments, the IL-11 binding receptor lacks amino acid sequence corresponding to the intracellular domain of IL-11Rα and/or the transmembrane domain of IL-11Rα. In some embodiments the IL-11 binding receptor lacks amino acid sequence corresponding to the stalk region of IL-11Rα. In some embodiments the IL-11 binding receptor lacks amino acid sequence corresponding to D1 of IL-11Rα.

In some embodiments, the IL-11 binding receptor of the present invention preferably lacks amino acid sequence corresponding to regions of IL-11Rα other than positions 112 to 317 of SEQ ID NO:18. In some embodiments the IL-11 binding receptor lacks amino acid sequence corresponding to positions 27 to 110 of SEQ ID NO:18.

In some embodiments, the IL-11 binding receptor may comprise further functional amino acid sequences. For example, the IL-11 binding receptor may comprise amino acid sequence(s) to facilitate expression, folding, trafficking, processing, purification or detection of the IL-11 binding receptor. For example, the IL-11 binding receptor may comprise a sequence encoding a protein tag, a g a FLAG, His, (e.g. 6×His), Myc, GST, MBP, HA, E, or Biotin tag, optionally at the N- or C-terminus.

In some embodiments, the IL-11 binding receptor of the present invention comprises, or consists of, an amino acid sequence having at least 60%, 65%, 70%, 75%, 80%, 85%, 86%, 87%, 88%, 89%, 90%, 91%, 92%, 93%, 4%, 95%. 98%. 97%, 98%, 99%, or 100% sequence identity to the amino acid sequence of SEQ ID NO 1 or 2.

IL-11 binding receptors according to the present invention may comprise or consist of an amino acid sequence that has a high percentage sequence identity to one or more of the nucleic acid sequences described herein, or nucleic acid sequence encoding the same amino acid sequence as a result of codon degeneracy. For example, IL-11 binding receptors according to the present invention include IL-11 binding receptors encoded by a nucleic acid sequence having at least 70%, more preferably one of at least 75%, 80%, 85%, 86%, 87%, 88%, 89%, 90%, 91%, 92%, 93%, 94%, 95%, 96%, 97%, 98%, 99%, or 100%, sequence identity to the IL-11 binding receptor encoded by the nucleic acid sequence of SEQ ID NO:9 or 10, or nucleic acid sequence encoding the same amino acid sequence as SEQ ID NO:9 or 10 as a result of codon degeneracy.

IL-11 binding receptors according to the present invention may be detectably labelled or, at least, capable of detection. For example, the IL-11 binding receptor may be labelled with a radioactive atom or a coloured molecule or a fluorescent molecule or a molecule which can be readily detected in any other way. Suitable detectable molecules include fluorescent proteins, luciferase, enzyme substrates, radio labels and binding moieties. Labeling may be by conjugation to the IL-11 binding receptor. The IL-11 binding receptor may be directly labelled with a detectable label or it may be indirectly labelled. In some embodiments, the label may be selected from: a radio-nucleotide, positron-emitting radionuclide (e.g., for positron emission tomography (PET)), MRI contrast gent or fluorescent label.

IL-11 binding receptors according to the present invention may be conjugated to a drug moiety, e.g. a cytotoxic small molecule. Such conjugates are useful for the targeted killing of calls expressing the antigen molecule.

### Functional Properties of the IL-11 Binding Receptors

The IL-11 binding receptors of the present invention may be characterised by reference to certain functional properties, in particular, an IL-11 binding receptor according to the present invention may possess one or more of the following properties:
a) Specific binding to IL-11 (e.g. human IL-11 and/or mouse IL-11);
b) Inhibition of interaction between IL-11 and IL-11Rα;
c) Inhibition of interaction between IL-11 and gp130;
d) Inhibition of interaction between IL-11 and IL-11Rα:gp130 receptor complex;
e) Inhibition of interaction between IL-11:IL-11Rα complex and gp130;
f) Inhibition of signalling mediated by IL-11;
g) Inhibition of signalling mediated by binding of IL-11 to IL-11Rα:gp130 receptor complex;
h) Inhibition of signalling mediated by binding of IL-11:IL-11Rα complex to gp130 (i.e. IL-11 trans signalling);
i) Inhibition of fibroblast proliferation;
j) Inhibition of myofibroblast generation from fibroblast;
k) Inhibition or a pathological process mediated by IL-11;
l) Inhibition of fibrosis;
m) Inhibition of gene or protein expression in fibroblasts of one or more of collagen, fibronectin, periostin, IL-6, IL-11, αSMA, TIMP1, MMP2, e.g. following stimulation with a profibrotic factor;
n) Inhibition of extracellular matrix production by fibroblasts
o) Inhibition of proliferation and/or survival of cells of a cancer;
p) Inhibition of tumour growth.

An IL-11 binding receptor which is capable of binding to IL-11 preferably binds IL-11 with greater affinity, and/or with greater duration than it binds to proteins other than IL-11.

In some embodiments the present IL-11 binding receptors may bind with greater affinity to IL-11 than to one or more other members of the IL-6 cytokine family. In same embodiments, the present IL-11 binding receptors may bind with greater affinity to IL-11 than to one or mare of IL-6, leukemia inhibitory factor (LIF) oncostatin M (OSM), cardiotrophin-1 (CT-1), ciliary neurotrophic factor (CNTF), and cardiotrophin-like cytokine (CLC).

In some embodiments, the extent of binding to a protein other than IL-11 is less then about 10% of the binding of the IL-11 binding receptor to IL-11 as measured, e.g., by ELISA, SPR, Bio-Layer interferometry (BLI), MicroScale Thermophoresis (MST), or by a radioimmunoassay (RIA). Alternatively, the binding specificity for IL-11 nay be reflected in terms of binding affinity, were the IL-11 binding receptor of the present invention binds to IL-11 with a KD that is at least 0.1 order of magnitude (i.e. 0.1×10n, where n is an integer representing the order of magnitude) greater than the KD towards another, non-target molecule. This may optionally be one of at least 0.2, 0.3, 0.4, 0.5, 0.6, 0.7, 0.8, 0.9, 1.0, 1.5, or 2.0.

Binding affinity may be expressed in terms of dissociation constant (KD). Binding affinity can be measured by methods known in the art, such as by ELISA (for example, as described in Antibody Engineering, vol. 1 (2nd Edn), Springer Protocols. Springer (2010), Part V, pp 657-R365). Surface Plasmon Resonance (SPR; see e.g. Hearty et al., Methods Mot Biol (2012) 907:411-442; or Rich et al. Anal Biochem. 2008 Feb. 1; 373(1):112-20). Bio-layer interferometry (see e.g. Lad et al. (2015) J Biomol Screen 20(4): 498-507; or Concepcion et al., Comb Chem High Throughput Screen. 2009 September; 12(8):791-800), MicroScale Thermophoresis (MST) analysis (see e.g. Jerabek-Willemsen et al., Assay Drug Dev Technol. 2011 August; 9(4): 342-353), or by a radiolabelled antigen binding assay (RIA).

In some embodiments, the IL-11 binding receptor according to the present invention binds to IL-11 with a KD of 5 μM or less, preferably one of ≤1 μM, ≤500 nM, ≤100 nM, ≤75 nM, ≤50 nM, ≤40 nM, ≤30 nM, ≤20 nM, ≤15 nM, ≤12.5 nM, ≤10 nM, ≤9 nM, ≤8 nM, ≤7 nM, ≤6 nM, ≤5 nM, ≤4 nM, ≤3 nM, ≤2 nM, ≤1 nM, ≤500 pM.

In some embodiments, the IL-11 binding receptor according to the present invention binds to IL-11 with an affinity of binding (e.g. as determined by ELISA) of EC50=1000 ng/ml or less, preferably one of ≤900 ng/ml, ≤800 ng/ml, ≤700 ng/ml, ≤600 ng/ml, ≤500 ng/ml, ≤400 ng/ml, ≤300 ng/ml, ≤200 ng/ml, ≤100 ng/ml, ≤90 ng/ml, ≤80 ng/ml, ≤70 ng/ml, ≤60 ng/ml, ≤50 ng/ml, ≤40 ng/ml, ≤30 ng/ml, ≤20 ng/ml, ≤15 ng/ml, ≤10 ng/ml, ≤7.5 ng/ml, ≤5 ng/ml, ≤2.5 ng/ml, or ≤1 ng/ml.

The IL-11 binding receptors according to the present invention inhibit IL-11 mediated signalling. Herein, ‘inhibition’ refers to a reduction, decrease or lessening relative to a control condition. For example, inhibition of a process by an IL-11 binding receptor refers to a reduction, decrease or lessening of the extent/degree of that process in the absence of the IL-11 binding receptor, and/or in the presence of an appropriate control receptor.

The skilled person is able to identify an appropriate control condition for a given assay. For example, a control receptor may be a receptor directed against a target protein which is known not to have a role involved in the property being investigated in the assay.

Inhibition may herein also be referred to as neutralisatlon or antagonism. That is an IL-11 binding receptor which is capable of inhibiting a function or process (e.g. interaction, signalling or other activity mediated by IL-11 or an IL-11-containing complex) may be said to be a ‘neutralising’ or ‘antagonist’ IL-11 biding receptor with respect to the relevant function or process. For example, IL-11 binding receptor which is capable of inhibiting IL-11 mediated signalling may be referred to as an IL-11 binding receptor which is capable of neutralising IL-11 mediated signalling, or may be referred to as an antagonist of IL-11 mediated signalling.

IL-11 binding receptors according to the present invention preferably compete for binding to IL-11 with one or more naturally occurring receptors for IL-11, e.g. gp130, IL-11Rα, or a variant/isoform/homologue or IL-11 binding fragment of gp130 or IL-11Rα. In some embodiments, an IL-11 binding receptor according to the present invention is a competitive inhibitor of binding of one of more of gp130, IL-11Rα, end variants/isoforms/homologues and IL-11 binding fragments of gp30 and/or IL-11Rα. That is, in some embodiments, the IL-11 binding receptor is capable of inhibiting interaction between IL-11 and one or more naturally occurring receptors for IL-11.

As used herein, a ‘naturally occurring receptor’ is a receptor which is found in nature. A naturally occurring receptor and/or the constituent peptides/polypeptides thereof may the product of transcription. mRNA processing (e.g. splicing), translation, and post-translational processing (e.g. proteolytic cleavage, glycosylation) from endogenous nucleic acid of a given host species.

In some embodiments, the IL-11 binding receptor according to the present invention binds to IL-11 in the region bound by one or more naturally occurring receptors for IL-11. In some embodiments, the IL-11 binding receptor binds to the same region, or overlapping region, of IL-11 as the region bound by one or more naturally occurring receptors for IL-11.

The ability of an IL-11 binding receptor to compete with a naturally occurring receptor for IL-11 (e.g. gp130, IL-11Rα, or variants/isoforms/homologues or IL-11 binding fragments of gp13 or IL-11Rα) for binding to IL-11 (i.e. inhibit interaction between IL-11 and the naturally occurring receptor), can be determined for example by analysis of Interaction between IL-11 and the naturally occurring receptor in the presence of or following incubation of IL-11, or IL-11 and the naturally occurring receptor, with the IL-11 binding receptor.

An example of a suitable assay to determine whether a given IL-11 binding receptor competes with gp130, IL-11Rα, or a variant/isoform/homologue or IL-11 binding fragment of gp130 or IL-11Rα for binding to IL-11 is a competitive binding assay, such as a competition ELISA assay.

An IL-11 binding receptor which is capable of Inhibiting a given interaction (e.g. between IL-11 and gp130, or between IL-11 and IL-11Rα, or between IL-11 and IL-11Rα:gp130, or between IL-11:IL-11Rα and gp130) is identified by the observation of a reduction/decrease in the level of interaction between the interaction partners in the presence of—or following incubation of one or both of the interaction partners with—the IL-11 binding receptor, as compared to the level of interaction in the absence of the IL-11 binding receptor (or in the presence of an appropriate control receptor). Suitable analysis can be performed in vitro, e.g. using recombinant interaction partners or using cells expressing the Interaction partners. Cells expressing interaction partners may do so endogenously, or may do so from nucleic acid introduced into the cell. For the purposes of such assays, one or both of the interaction partners and/or the IL-11 binding receptor may be labelled or used in conjunction with a detectable entity for the purposes of detecting and/or measuring the level of interaction.

In some embodiments, the IL-11 binding receptor according to the present invention inhibits interaction between human IL-11 and human gp130. In some embodiments, the IL-11 binding receptor inhibits interaction between human IL-11 In complex with human IL-11Rα (i.e. human IL-11:IL-11Rα) and human gp130. In some embodiments, the IL-11 binding receptor inhibits interaction between human IL-11 and human gp130-IL-11Rα receptor complex. In some embodiments, the IL-11 binding receptor inhibits interaction between human IL-11:IL-11Rα and human gp130:IL-11Rα receptor complex.

Such assays are also useful to determine whether an IL-11 binding receptor binds to the same region or overlapping region of IL-11 as the region bound by one or more naturally occurring receptors for IL-11. That is, observation of a reduction/decrease in t level of interaction between IL-11 or IL-11:IL-11Rα and e.g. gp130, IL-11Rα, or IL-11Rα:gp130 in the presence of—or following incubation of one or both of the interaction partners with—the IL-11 binding receptor, as compared to the level of interaction in the absence of the IL-11 binding receptor suggests that IL-11 binding receptor binds to the same region or overlapping region of IL-11 as the region bound by one or more naturally occurring receptors for IL-11.

Whether an IL-11 binding receptor according to the present invention binds to IL-11 in the same or same region or overlapping region of IL-11 as the region bound by a naturally occurring receptor for IL-11 can also be determined by analysis of interaction using various methods well known in the art, including X-ray co-crystallography analysis of receptor-ligand complexes, peptide scanning, mutagenesis mapping, hydrogen-deuterium exchange analysis by mass spectrometry, phage display and proteolysis-based ‘protection’ methods. Such methods are described, for example, in Gershoni et al. BioDrugs. 2007, 21(3):145-156, incorporated by reference hereinabove. The region of IL-11 bound by naturally occurring receptors for IL-11 can also be determined by reference e.g. to Griffin et al, 2013, Cytokine 63(3) 267 and/or Putoczki at al., 2014, Acta Crystallogr D Biol Crystallogr 70(Pt 9):2277-85.

Ability of an IL-11 binding receptor inhibit interaction between two interaction partners can also be determined by analysis of the downstream functional consequences of the interaction, e.g. receptor signalling. For example, downstream functional consequences of interaction between IL-11 and IL-11Rα:gp130 or between IL-11:IL-11Rα and gp130 may include proliferation of fibroblasts, myofibroblast generation from fibroblasts, or gene or protein expression of one or more of collagen, fibronectin, periostin, IL-6, IL-11, αSMA, TIMP1, MMP2.

Fibroblasts according to the present disclosure may be derived from any issue, including liver, lungs, kidney, heart, blood vessels, eye, skin, pancreas, spleen, bowel (e.g. large or small intestine), brain, and bone marrow. In particular embodiments, for the purposes of analysis of the IL-11 binding receptor, the fibroblasts may be cardiac fibroblasts (e.g. atrial fibroblasts), skin fibroblasts, lung fibroblasts, kidney fibroblasts or liver fibroblasts. Fibroblasts may be characterised by gene or protein expression of one or more of COL1A, ACTA2, prolyl-4-hydroxylase, MAS516, and FSP1.

Gene expression can be measured by various means known to those skilled in the art, for example by measuring levels of mRNA by quantitative real-time PCR (qRT-PCR), or by reporter-based methods. Similarly, protein expression can be measured by various methods well known in the art, e.g. by antibody-based methods, for example by western blot, immunohistochemistry, immunocytochemistry, flow cytometry, ELISA, ELISPOT, or reporter-based methods.

In some embodiments, the IL-11 binding receptor according to the present invention may inhibit protein expression of one or more markers of fibrosis, e.g. protein expression of one or more of collagen, fibronectin, periostin, IL-6, IL-11, αSMA, TIMP1, MMP2.

The ability of an IL-11 binding receptor to Inhibit interaction between IL-11 and IL-11Rα:gp130 can, for example, be analysed by stimulating fibroblasts with TGFβ1, incubating the cells in the presence of the IL-11 binding receptor and analysing the proportion of cells having αSMA-positive phenotype after a defined period of time in such example, inhibition of interaction between IL-11 and IL-11Rα:gp130 can be identified by observation of a lower proportion of cells having an αSMA-positive phenotype as compared to positive control condition in which cells are treated with TGFβ1 In the absence of the IL-11 binding receptor (or in the presence of an appropriate control receptor), or in the presence of an appropriate control receptor.

Such assays are also suitable for analysing the ability of the IL-11 binding receptor to inhibit IL-11-mediated signalling.

In some embodiments, the IL-11 binding receptor according to the present invention is capable of inhibiting interaction between IL-11 and gp130 to less tan 100%. e.g. one of 99% or less, 95% or less, 90% or less, 85% or less, 75% or less, 70% or less, 65% or less, 60% or less, 56% or less, 50% or less, 45% or less, 40% or less, 35% or less, 30% or less, 25% or less, 20% or less, 15% or less, 10% or less, 5% or less, or 1% or less of the level of interaction between IL-11 and gp130 in the absence of the IL-11 binding receptor (or in the presence of an appropriate control receptor). In some embodiments, the IL-11 binding receptor according to the present invention is capable of inhibiting interaction between IL-11 and gp130 to less than 1 times, e.g. one of ≤0.99 times, ≤0.95 times, ≤0.9 times, ≤0.85 times, ≤0.8 times, ≤0.85 times, ≤0.75 times, ≤0.7 times, ≤0.66 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of interaction between IL-11 and gp130 in the absence of the IL-11 binding receptor (or in the presence of an appropriate control receptor).

In some embodiments, the IL-11 binding receptor according to the present invention is capable of inhibiting interaction between IL-11 and IL-11Rα to less then 100%, e.g. one of 99% or less, 95% or less, 90% or less, 85% or less, 75% or less, 70% or less, 65% or less, 60% or lees, 55% or less, 50% or less, 45% or less, 40% or less, 35% or less, 30% or less, 25% or less, 20% or less, 15% or less, 10% or less, 5% or less, or 1% or less of the level of Interaction between IL-11 and IL-11Rα in the absence of the IL-11 binding receptor (or in the presence of an appropriate control receptor). In some embodiments, the IL-11 binding receptor according to the present invention is capable of inhibiting interaction between IL-11 and IL-11Rα to less then 1 times, e.g. one of ≤0.99 times, ≤0.95 times, ≤0.9 times, ≤0.85 times, ≤0.8 times, ≤0.85 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of interaction between IL-11 and IL-11Rα in the absence of the IL-11 binding receptor (or in the presence of an appropriate control receptor).

In some embodiments, the IL-11 binding receptor according to the present invention is capable of inhibiting interaction between IL-1 and IL-11Rα:gp130 to less than 100%, e.g. one of 99% or less, 95% or less, 90% or less, 85% or less, 75% or less, 70% or less, 65% or less, 30% or less, 55% or less, 50% or less, 45% or less, 40% or less, 35% or less, 30% or less, 25% or less, 20% or less, 15% or loss, 10% or less, 5% or less, or 1% or less of the level of interaction between IL-11 and IL-11Rα:gp130 in the absence of the IL-11 binding receptor (or in the presence of an appropriate control receptor) in some embodiments, the IL-11 binding receptor according to the present invention Is capable of inhibiting interaction between IL-11 and IL-11Rα:gp130 to less than 1 times, e.g. one of ≤0.99 times, ≤0.95 times, ≤0.9 times, ≤0.85 times, ≤0.8 times, ≤0.85 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 tines the level of interaction between IL-11 and IL-11Rα:gp130 in the absence d the IL-11 binding receptor (or in the presence of an appropriate control receptor).

In some embodiments, the IL-11 binding receptor according to the present invention is capable of Inhibiting interaction between IL-11:IL-11Rα complex and gp130 to less than 100%. e.g. one of 99% or less, 95% or less. 90% or less, 85% or less, 75% or less, 70% or less, 65% or less, 60% or less, 55% or less, 50% or less, 45% or less, 40% or less, 38% or less, 30% or less, 25% or less, 20% or less, 15% or less, 10% or less, 5% or less, or 1% or less of the level of interaction between IL-11:IL-11Rα complex and gp130 in the absence of the IL-11 binding receptor (or in the presence of an appropriate control receptor). In some embodiments, the IL-11 binding receptor is capable of Inhibiting interaction between IL-11:IL-11Rα complex and gp130 to lese than t times, e.g. one of ≤0.99 times, ≤0.95 times, ≤0.9 times, ≤0.85 times, ≤0.8 times, ≤0.85 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of interaction between IL-11:IL-11Rα complex and gp130 in the absence of the IL-11 binding receptor for in the presence of an appropriate control receptor).

Inhibition of IL-11 mediated signalling can be analysed using ³H-thymidine incorporation and/or Ba/F3 cell proliferation assays such as those described in e.g. Curtis et al. Blood, 1997, 90(11) and Karpovich et al. Mol. Hum. Reprod. 2003 9(2): 75-80. Ba/F3 cells co-express IL-11Rα and gp130.

As used herein, ‘IL-11 mediated signalling’ and/or processes mediated by IL-11 includes signalling mediated by fragments of IL-11 and polypeptide complexes comprising IL-11 or fragments thereof, IL-11 mediated signalling may be signalling mediated by human IL-11 and/or mouse IL-11. Signalling mediated by IL-11 may occur following binding of IL-11 or an IL-11 containing complex to a receptor to which IL-11 or said complex binds.

In some embodiments, IL-11 binding receptors according to the present invention are capable of inhibiting the biological activity of IL-11 or an IL-11-containing complex. In some embodiments, the IL-11 binding receptor binds to IL-11 or the IL-11-containing complex in a region which is important for binding to a receptor for the IL-11 or IL-11-containing complex, e.g. gp130 or IL-11Rα, and thereby disrupts binding to and/or signalling through the receptor.

In some embodiments, the IL-11 binding receptor according to the present invention is an antagonist of one or more signalling pathways which are activated by signal transduction through receptors comprising IL-11Rα and/or gp130, e.g. IL-11Rα:gp130. In some embodiments, the IL-11 binding receptor is capable of inhibiting signalling through ore or more immune receptor complexes comprising IL-11Rα and/or gp130, e.g. IL-11Rα:gp130.

In some embodiments, the IL-11 binding receptor according to the present invention is capable of inhibiting IL-11-mediated signalling to less than 100%, e.g. one of 99% or less, 95% or less, 90% or less, 85% or less, 75% or less, 70% or less, 65% or less, 60% or less, 55% or less, 50% or less, 45% or less, 40% or less 35% or less, 30% or less, 25% or less 20% or less, 15% or less, 10% or less, 5% or less, or 1% or less of the level of signalling in the absence of the IL-11 binding receptor (or in the presence of an appropriate control receptor). In some embodiments, the IL-11 binding receptor is capable of reducing IL-11 mediated signalling to less than 1 times, e.g. one of ≤0.99 times, ≤0.95 times, ≤0.9 times, ≤0.85 times, ≤0.8 times, ≤0.85 times, ≤0.75 times, ≤0.7 times, ≤0.65 times, ≤0.6 times, ≤0.55 times, 0.5 times, ≤0.45 times, 0.04 times, ≤0.35 times, ≤0.3 times, ≤0.25 times, ≤0.2 times, ≤0.15 times, ≤0.1 times the level of signalling in the absence of the IL-11 binding receptor (or in the presence of an appropriate control receptor).

In some embodiments, the IL-11 mediated signalling may be signalling mediated by binding of IL-11 to IL-11α:gp130 receptor. Such signalling can be analysed e.g. by treating cells expressing IL-11Rα and gp130 with IL-11, or by stimulating IL-11 production in cells which express IL-11Rα and gp130.

The IC₅₀ for IL-11 binding receptor for inhibition of IL-11 mediated signalling may be determined, e.g. by culturing Ba/F3 cells expressing IL-11Rα and gp130 in the presence of human IL-11 and the IL-11 binding agent, and measuring ³H-thymidine incorporation into DNA.

In some embodiments, the IL-11 binding receptor of the present invention may exhibit an IC₅₀ of 10 μg/ml or less, preferably one of ≤5 μg/ml, ≤4 μg/ml, ≤3.5 μg/ml, ≤3 μg/ml, ≤2 μg/ml, ≤1 μg/ml, ≤0.9 μg/ml, ≤0.8 μg/ml, ≤0.7 μg/ml, ≤0.6 μg/ml, or ≤0.5 μg/ml in such an assay.

In some embodiments, the IL-11 mediated signalling may be signalling mediated by binding of IL-11:IL-11Rα complex to gp130. In some embodiment, the IL-11:IL-11Rα complex may be soluble, e.g. complex of extracellular domain of IL-11Rα and IL-11, or complex of soluble IL-11Rα isoform/fragment, and IL-11. In some embodiments, the soluble IL-11Re is a soluble (secreted) isoform of IL-11Rα, or is the liberated product of proteolytic cleavage of the extracellular domain of cell membrane bound IL-11Rα.

In some embodiments, the IL-11:IL-11Rα complex may be cell-bound, e.g. complex of cell-membrane bound IL-11Rα and IL-11. Signaling mediated by binding of IL-11:IL-11Rα complex to gp130 can be analysed by treating cells expressing gp130 with IL-11:IL-11Rα complex, e.g. recombinant fusion protein comprising IL-11 joined by a peptide linker to the extracellular domain of IL-11Rα (e.g. Hyper IL-11 as described herein).

In some embodiments, the IL-11 binding receptor according to the present invention is capable of inhibiting signalling mediated by binding of IL-11:IL-11Rα complex to gp130, and is also capable of inhibiting signalling mediated by binding of IL-11 to IL-11Rα:gp130 receptor.

In some embodiments, the IL-11 binding receptor is capable of inhibiting fibroblast proliferation. Proliferation of fibroblast can be determined by analysing cell division over a period of time. Cell division for a given population of fibroblasts can be analysed, for example, by in vitro analysis of incorporation of ³H-thymidine or by CFSE dilution assay, e.g. as described in Fulcher and Wong, Immunol Cell Biol (1999) 77(6): 559-564, hereby incorporated by reference in entirety. Proliferating cells (e.g. proliferating fibroblasts) may also be identified by analysis of incorporation of 5-ethynyl-2′deoxyuridine (EdU) by an appropriate assay, as described e.g. in Buck at al., Biotechniques, 2008 June; 44(7):927-9, and Sal and Mitchison, PNAS USA 2008 Feb. 19; 106(7): 2415-2420, both hereby incorporated by reference in their entirely.

Fibroblasts according to the present disclosure may be derived from any tissue, including liver, lungs, kidney, heart, blood vessels, eye, skin, pancreas, spleen, bowel (e.g. large or small intestine), brain, and bone marrow. In particular embodiments, for the purposes of analysis of the IL-11 binding receptor, the fibroblasts may be cardiac fibroblasts (e.g. atrial fibroblasts), skin fibroblasts, lung fibroblasts, kidney fibroblasts or liver fibroblasts.

In some embodiments, the IL-11 binding receptor according to the present invention is capable of inhibiting fibroblast proliferation to less than 100%, e.g. one of 99% or less, 95% or less, 90% or less, 85% or less, 75% or less, 70% or less, 65% or less, 0% or less, 55% or less, 50% or less, 45% or less, 40% or less, 35% or less, 30% or less, 25% or less, 20% or less, 15% or less, 10% or les, 5% or less, or 1% or less of the level of fibroblast proliferation in the absence of the IL-11 binding receptor (or in the presence of an appropriate control receptor). In some embodiments, the IL-11 binding receptor is capable of reducing fibroblast proliferation to less than 1 times, e.g. one of ≤0.99 times, ≤0.95 times, ≤0.9 times, ≤0.85 times, ≤0.8 times, ≤0.85 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of fibroblast proliferation in the absence of the IL-11 binding receptor (or in the presence at an appropriate control receptor).

In some embodiments, the IL-11 binding receptor according to the present invention is capable of inhibiting a pathological process mediated by IL-11, e.g. following stimulation with a profibrotic factor (e.g. TGFβ1). Pathological processes mediated by IL-11 include fibrosis, and can be evaluated either in vitro or in vivo.

In some embodiments, the IL-11 binding receptor according to the present invention is capable of inhibiting fibrosis. Fibrosis may be of a particular tissue or several tissues, e.g. liver, lung, kidney, heart, blow vessel, eye, skin, pancreas, spleen, bowel (e.g. large or small intestine), brain, or bone marrow. Fibrosis may be measured by means welt known to the skilled person, for example by analysing gene or protein expression of one or more myofibroblast markers and/or gone or protein expression of one or more markers of fibrosis in a given tissue or tissues.

Myofibroblast markers may include one or more of increased αSMA, vimentin, palladin, cordilin or desmin. Markers of fibrosis include increased level d collagen, fibronectin, periostin, IL-6, IL-11, αSMA, TIMP1 and MMP2, extracellular matrix components, number/proportion of myofibroblasts, and organ weight.

Inhibition of fibrosis can be measured in vitro or in vivo. For example, whether an IL-11 binding receptor is capable of inhibiting fibrosis in a given tissue can be analysed in vitro by treating fibroblasts derived from that tissue with a profibrotic stimulus, and then analysing whether the IL-11 binding receptor can reduce myofibroblast generation from the fibroblasts (or e.g. some other marker of fibrosis). Whether an IL-11 binding receptor is capable of inhibiting fibrosis can be analysed in vivo, for example, by administering the IL-11 binding receptor to a subject (e.g. a subject that has been exposed to a profibrotic stimulus), and analysing tissue(s) for one or more markers of fibrosis.

In some embodiments, the IL-11 binding receptor according to the present invention is capable of inhibiting fibrosis to less than 100%, e.g. one of 99% or less, 95% or less, 90% or less, 85% or less, 75% or less, 70% or less, 65% or less, 60% or less, 55% or less, 50% or les, 45% of less. 40% or less, 35% or less, 30% or less. 25% or less, 20% or less, 15% or less, 10% or less, 5% or less, or 1% or less of the level of fibrosis in the absence of the IL-11 binding receptor (or in the presence of an appropriate control receptor. In some embodiments, the IL-11 binding receptor is capable of reducing fibrosis to less than 1 times, e.g. one of ≤0.99 times, ≤0.95 times, ≤0.9 times, ≤0.85 times, ≤0.8 times, ≤0.85 times, ≤0.75 times, ≤0.7 times, ≤0.65 times, ≤0.6 times, ≤0.55 times, ≤0.5 times, ≤0.45 times, ≤0.4 times, ≤0.35 times, ≤50.3 times, ≤0.25 times, ≤0.2 times, ≤0.15 times, ≤0.1 times the level of fibrosis in the absence of the IL-11 binding receptor in the presence of an appropriate control receptor).

In some embodiments, the IL-11 binding receptor according to the present invention is capable of inhibiting myofibroblast generation from fibroblasts, e.g. following exposure of the fibroblasts to profibrotic factor. Myofibroblast generation from fibroblasts can be investigated by analysis for myofibroblast markers. A profibrotic factor according to the present disclosure may be e.g. TGFβ1, IL-11, IL-13, PDGF, ET-1, oncostatin M (OSM) or ANG2 (AngII).

In some embodiments, the IL-11 binding receptor is capable of inhibiting gene or protein expression in fibroblasts, or fibroblast-derived cells (e.g. myofibroblasts), of one or more of collagen, fibronectin, periostin, IL-6, IL-11, αSMA, TIMP1, MMP2, e.g. following stimulation with a profibrotic factor. In some embodiments, the IL-11 bending receptor Is capable of inhibiting gene or protein expression in fibroblasts, or fibroblast-derived cells (e.g. myofibroblasts), of one or more extracellular matrix components, e.g. following stimulation with a profibrotic factor.

In the experimental examples herein, myofibroblast generation from fibroblasts is analysed by measuring αSMA protein expression levels using Operetta High-Content Imaging System following stimulation of the fibroblasts with TGFβ1.

In some embodiments, the IL-11 binding receptor according to the present invention is capable of inhibiting myofibroblast generation from fibroblasts to less than 100%, e.g. one of 99% or less, 95% or less, 90% or less, 85% or less, 75% or less, 70% or less, 65% or less, 80% or less, 55% or less, 50% or less, 45% or less, 40% or less, 35% or less, 30% or less, 25% or less, 20% or less, 15% or less, 10% or less, 5% or less, or 1% or less of the level of myofibroblast generation from fibroblasts in the absence of the IL-11 binding receptor (or in the presence of an appropriate control receptor) in some embodiments, the IL-11 binding receptor is capable of reducing myofibroblast generation from fibroblasts to leas than 1 times, e.g. one of ≤0.99 times, ≤0.95 times, ≤0.9 times, ≤0.85 times, ≤0.8 times, ≤0.85 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of myofibroblast generation from fibroblasts in the absence of the IL-11 binding receptor (or in the presence of an appropriate control receptor).

In some embodiments, the IL-11 binding receptor according to the present invention is capable of inhibiting gene or protein expression in fibroblasts of one or more of collagen, fibronectin, periostin, IL-6, IL-11, αSMA, TIMP1, MMP2, e.g. following stimulation with a profibrotic factor (e.g. TGFβ1). In some embodiments, the IL-11 binding receptor according to the present invention is capable of inhibiting gene or protein expression to less than 100%, e.g. one of 99% or less, 95% or less, 90% or less, 85% or less, 75% or less, 70% or less, 65% or less, 60% or less 5 or less, 50% or les, 45% or less, 40% or less, 35% or less, 30% or less, 25% or less, 20% or less, 16% or less, 10% or less, 5% or less, or 1% or less of the level of gene or protein expression in the absence of the IL-11 binding receptor (or in the presence of an appropriate control receptor). In some embodiments, the IL-11 binding receptor is capable of reducing gene or protein expression to less than 1 times, e.g. one of ≤0.99 times, ≤0.95 times, ≤0.9 times, ≤0.85 times, ≤0.8 times, ≤0.85 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, 0.2 times, ≤0.15 times, ≤0.1 times the level of gene or protein expression in the absence of the IL-11 binding receptor (or in the presence of an appropriate control receptor).

In some embodiments, the IL-11 binding receptor according to the present invention is capable of imbibing extracellular matrix production by fibroblasts, e.g. following stimulation with a profibrotic factor (e.g. TGFβ1) Extracellular matrix production can be evaluated, or example, by measuring the level of an extracellular matrix component. Extracellular matrix components according to the present invention include e.g. proteoglycan, heparan sulphate, chondroitin sulphate, keratan sulphate, hyaluronic acid, collagen, periostin, fibronectin, vitronectin, elastin, fibronectin, laminin, nidogen, gelatin and aggrecan.

In some embodiments, the IL-11 binding receptor according to the present invention is capable of inhibiting extracellular matrix production by fibroblasts to less than 100%, e.g. one of 99% or less, 95% or less, 90% or less 85% or less, 75% or less, 70% or less, 65% or less, 60% or less, 55% or less, 50% or less, 45% or less. 40% or less, 35% or less, 30% or less, 25% or less, 20% or less, 15% or less, 10% or less, 5% or less, or 1% or less of the level of extracellular matrix production by fibroblasts in the absence of the IL-11 binding receptor (or in the presence of an appropriate control receptor). In some embodiments, the IL-11 binding receptor is capable of reducing extracellular matrix production by fibroblasts to loss than 1 times, e.g. one of ≤0.99 times, ≤0.95 times, ≤0.9 times, ≤0.85 times, ≤0.8 times, ≤0.85 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of extracellular matrix production in the absence of the IL-11 binding receptor (or in the presence of an appropriate control receptor).

In some embodiments, the IL-11 binding receptor according to the present invention is capable of inhibiting proliferation and/or survival of cells of a cancer. The skilled person is able to determine whether an IL-11 binding receptor is capable of inhibiting proliferation and/or survival of cells of a cancer for example by analysing the effect of the IL-11 banding receptor on cells of the cancer. For example, proliferation of cells can be measured as described herein, e.g. by ³H thymidine Incorporation or CFSE dilution assays. Cell survival can be analysed by measuring cells for markers of cell viability/cell death following treatment with the IL-11 binding receptor.

In some embodiments, the IL-11 binding receptor according to the present invention is capable of inhibiting proliferation and/or survival of cells of a cancer to less than 100% e.g. one of 99% or less 95% or lees, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of proliferation and/or survival of cells of a cancer in the absence of the IL-11 binding receptor (or in the presence of an appropriate control receptor). In some embodiments, the IL-11 binding receptor is capable of reducing proliferation and/or survival of cells of a cancer to less than 1 times, e.g. one of ≤0.99 times, ≤0.95 times, ≤0.9 times, ≤0.85 times, ≤0.8 times, ≤0.85 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of proliferation and/or survival of cells of a cancer in the absence of the IL-11 binding receptor (or in the presence of an appropriate control receptor).

In some embodiments, the IL-11 binding receptor according to the present invention is capable of Inhibiting tumour growth to less than 100%. e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of tumour growth in the absence of the IL-11 binding receptor (or in the presence of an appropriate control receptor). In same embodiments, the IL-11 binding receptor is capable of reducing tumour growth to less than 1 times, e.g. one of ≤0.99 times, ≤0.95 times, ≤0.9 times, ≤0.85 times, ≤0.8 times, ≤0.85 times, 50.75 times, ≤0.7 times, ≤0.05 times, ≤0.8 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of tumour growth in the absence of the IL-11 binding receptor (or in the presence of an appropriate control receptor).

### Therapeutic Applications

IL-11 binding receptors according to the present invention and compostions comprising such agents may be provided for use in methods of medical treatment or prevent of a disease/disorder, or alleviation of the symptoms of a disease/disorder. The IL-11 binding receptors of the present invention may be administered to subjects having a disease/condition in need of treatment, and/or to subjects at risk of such developing or contracting the disease/disorder.

Treatment, prevention or alleviation of fibrosis according to the present invention may be of fibrosis that is associated with an upregulation of IL-11 and/or IL-11Rα, e.g. an upregulation of IL-11 in cells or issue in which the disease/disorder occurs or may occur, or upregulation of extracellular IL-11 or IL-11Rα. In some embodiments, IL-11 or IL-11Rα expression is locally or systemically upregulated in the subject.

Treatment or alleviation of a disease/disorder may be effective to prevent progression of the disease/disorder, e.g. to prevent worsening of the condition or to slow the rate of development. In some embodiments treatment or alleviation may lead to an improvement in the disease/disorder, e.g. a reduction in the symptoms of the disease/disorder or reduction in some other correlate of the severity/activity of the disease/disorder.

Prevention of a disease/disorder may refer to prevention of a worsening of the condition or prevention of the development of the disease/disorder, e.g. preventing an early stage disease/disorder developing to a later, chronic, stage.

The IL-11 binding receptors of the present invention are preferably able to bid to and inhibit the biological activity of IL-11 and IL-11-containing molecules/complexes (e.g. IL-11:IL-11Rα complex). Accordingly, the IL-11 binding receptors of the present invention find use in the treatment or prevention of diseases and disorders in which IL-11 and/or IL-11Rα is implicated in the pathology of the disease/disorder. That is, the IL-11 binding receptors of the present invention find use in the treatment or prevention of diseases and disorders associated with IL-11/IL-11R signalling.

In some embodiments, the disease/disorder may be associated with increased IL-11, IL-11Rα and/or gp130 gene or protein expression, e.g. as compared to the control (i.e. non-diseased) state. In some embodiments, the disease/disorder may be associated with an increased level of IL-11/IL-11R signalling as compared to the control state. In some embodiments, the disease/disorder may be associated with an increased level of signalling through ERK and/or STAT3 Pathways as compared to the control state. In some embodiments, the increased expression/activity of IL-11, IL-11Rα and/or gp130, and/or the increased level of IL-11/IL-11R signalling, may be observed in effector cells of the disease/disorder (e.g. for a cancer, the cancerous cells, in some embodiments, the increased expression/activity of IL-11, IL-11Rα and/or gp130, and/or the increased level of IL-11/IL-11R signalling, may be observed in cells other than the effector calls.

Signalling through ERK can be measured e.g. using an assay for ERK phosphorylation such as an assay described in Assay Guidance Manual: Phospho-ERK Assays. Kim E. Garbison, Beverly A. Heinz, Mary E. Lajiness, Jeffrey R. Weidner, and G. Sitta Sittampalam, Eli Lily & Company, Sittampalam G S, Coussen N P, Nelson H, et al. editors Bethesda (MD): Eli Lilly & Company and the National Center for Advancing Translational Sciences; 2004. Signalling through STAT3 can be measured e.g. using an assay for phosphorylation of STAT3, such as the Phospho-STAT3 (Tyr705) Cellular Assay Kit (Cisbio Assays).

In some embodiments, the treatment is of a disease/disorder for which a reduction in IL-11/IL-11R signalling is therapeutic. In some embodiments, the treatment is of a disease/disorder associated with excess ERK and/or STAT3 signalling. In some embodiments, the treatment is of a disease/disorder associated with excess proliferation or hyperactivation of fibroblasts, or associated with an excess of myofibroblasts.

In some embodiments, the treatment may be aimed at preventing or treating a disease/disorder by decreasing the number or proportion of myofibroblasts or αSMA-positive fibroblasts.

In some embodiments, the disease/disorder may be fibrosis, a fibrotic condition, or a disease/disorder characterised by fibrosis. As used herein, “fibrosis” refers to the formation of excess fibrous connective tissue as a result of the excess deposition of extracellular matrix components, for example collagen Fibrous connective tissue is characterised by having extracellular matrix (ECM) with a high collagen content. The collagen may be provided in strands or fibers, which may be arranged irregularly or aligned. The ECM of fibrous connective tissue may also include glycosaminoglycans.

As used herein, “excess fibrous connective tissue” refers to an amount of connective tissue at a given location (e.g. a given tissue or organ, or part of a given tissue or organ) which is greater than the amount of connective tissue present at that location in the absence of fibrosis, e.g. under normal, non-pathological conditions. As used herein “excess deposition of extracellular matrix components” refers to a level of deposition of one or more extracellular matrix components which is greater than the level of deposition in the absence of fibrosis, e.g. under normal, non-pathological conditions.

The cellular and molecular mechanisms of fibrosis are described in Wynn, J. Pathol. (2008) 214(2): 199-210, and Wynn and Ramalingam. Nature Medicine (2012) 18:1028-1040, which are hereby incorporated by reference in their entirety. The main cellular effectors of fibrosis are myofibroblasts, which produce a collagen-rich extracellular matrix.

In response to tissue injury, damaged cells and leukocytes produce pro-fibrotic factors such as TGFβ, IL-13 and PDGF, which activate fibroblasts to αSMA-expressing myofibroblasts, aid recruit myofibroblasts to the site of injury. Myofibroblasts produce a large amount of extracellular matrix, and are important mediators in aiding contracture and closure of the wound. However, under conditions of persistent infection or during chronic inflammation there can be overactivation and recruitment of myofibroblasts, and thus over-production of extracellular matrix components, resulting in the formation of excess fibrous connective tissue.

In some embodiments fibrosis may be triggered by pathological conditions, e.g. conditions, infections or disease states that lead to production of pro-fibrotic factors such as TGFβ1. In some embodiments, fibrosis may be caused by physical injury/stimuli, chemical injury/stimuli or environmental injury/stimuli. Physical injury/stimuli may occur dung surgery, e.g. iatrogenic causes. Chemical injury/stimuli may include drug induced fibrosis, e.g. following chronic administration of drugs such as bleomycin, cyclophosphamide, amiodarone, procainamide, penicillamine, gold and nitrofurantoin (Daba et al., Saudi Med J 2004 June; 25(6): 700-6) Environmental injury/stimuli may include exposure to asbestos fibres or silica.

Fibrosis can occur in many tissues of the body. For example, fibrosis can occur in the lung, liver (e.g. cirrhosis), kidney, heart, blood vessels, eye, skin, pancreas, spleen, bowel (e.g. large or small intestine), brain, and bone marrow. Fibrosis may also occur in multiple organs at once.

In embodiments herein, fibrosis may involve an organ of the gastrointestinal system. e.g. of the liver, small intestine, large intestine, or pancreas. In some embodiments, fibrosis may involve an organ of the respiratory system, e.g. the lungs. In embodiments, fibrosis may Involve an organ of the cardiovascular system. e.g. of the heart or blood vessels, in some embodiments, fibrosis may involve the skin. In some embodiments, fibrosis may involve an organ of the nervous system, e.g. the brain. In some embodiments, fibrosis may involve an organ of the urinary system, e.g. the kidneys. In some embodiments, fibrosis may involve an organ of the musculoskeletal system, e.g. muscle tissue.

In some preferred embodiments, the fibrosis is cardiac or myocardial fibrosis, hepatic fibrosis, or renal fibrosis. In some embodiments cardiac or myocardial fibrosis is associated with dysfunction of the musculature or electrical properties of the heart, or thickening of the wells or valves of the heart. In some embodiments fibrosis is of the atrium and/or ventricles of the heart. Treatment or prevention of atrial or ventricular fibrosis may help reduce risk or onset of atrial fibrillation, ventricular fibrillation, or myocardial infarction.

In some preferred embodiments hepatic fibrosis is associated with chronic liver disease or liver cirrhosis. In some preferred embodiments renal fibrosis is associated with chronic kidney disease.

Diseases/disorders characterised by fibrosis in accordance with the present invention include but are not limited to: respiratory conditions such as pulmonary fibrosis, cystic fibrosis, idiopathic pulmonary fibrosis, progressive massive fibrosis, scleroderma, obliterative bronchiolitis, Hermansky-Pudlak syndrome, asbestosis, silicosis, chronic pulmonary hypertension, AIDS associated pulmonary hypertension, sarcoidosis, tumor stroma in lung disease, and asthma; chronic liver disease, primary biliary cirrhosis (PBC), schistosomal liver disease, liver cirrhosis: cardiovascular conditions such as hypertrophic cardiomyopathy, dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertensive heart disease, arrhythmogenic right ventricular cardiomyopathy (ARVC), tubulointerstitial and glomerular fibrosis, atherosclerosis, varicose veins, cerebral infarcts; neurological conditions such as gliosis and Alzheimer's disease; muscular dystrophy such as Duchenne muscular dystrophy (DMD) or Becker's muscular dystrophy (BMD); gastrointestinal conditions such as Chron's disease, microscopic colitis and primary sclerosing cholangitis (PSC); skin conditions such as scleroderma, nephrogenic systemic fibrosis and cutis keloid; arthrofibrosis; Dupuytren's contracture; mediastinal fibrosis; retroperitoneal fibrosis; myelofibrosis; Peyronie's disease; adhesive capsulitis; kidney disease (e.g., renal fibrosis, nephritic syndrome, Alport's syndrome, HIV associated nephropathy, polycystic kidney disease, Fabry's disease, diabetic nephropathy, chronic glomerulonephritis, nephritis associated with systemic lupus); progressive systemic sclerosis (PSS); chronic graft versus host disease; diseases/disorders of the eye and associated processes, such as Grave's opthalmopathy, epiretinal fibrosis (e.g. diabetic retinopathy (DR)), glaucoma, subretinal fibrosis (e.g. associated with macular degeneration (e.g. wet age-related macular degeneration (AMD))), macular edema, drusen formation, post-surgical fibrosis (e.g. of the posterior capsule following cataract surgery, or of the blob following trabeculectomy for glaucoma), conjunctival fibrosis, subconjunctival fibrosis; arthritis; fibrotic pre-neoplastic and fibrotic neoplastic disease; and fibrosis induced by chemical or environmental insult (e.g., cancer chemotherapy, pesticides, radiation/cancer radiotherapy).

It will be appreciated that many of the diseases/conditions listed above are interrelated. For example, frosts of the ventricle may occur post myocardia Infarction, and is associated with DCM, HCM and myocarditis.

In particular embodiments, the disease/disorder may be one of pulmonary fibrosis, atrial fibrillation, ventricular fibrillation, hypertrophic cardiomyopathy (HCM), dilated cardiomyopathy (DCM), non-alcoholic steatohepatitis (NASH), cirrhosis, chronic kidney disease, scleroderma, systemic sclerosis, keloid, cystic fibrosis, Chron's disease, post-surgical fibrosis or retinal fibrosis, e.g. associated with wet age-related macular degeneration (AMD).

Fibrosis can lead directly or indirectly to, and/or increase susceptibility to development of, diseases/disorders. For example, more than 80% of hepatocellular carcinomas (HCCs) develop in fibrotic or cirrhotic livers (Affo et al. 2016, Annu Rev Pathol), suggesting an important role for liver fibrosis in the premalignant environment (PME) of the liver.

Accordingly, the antibodies/fragments of the present invention find use in methods for the treatment and prevention of diseases/disorders associated with fibrosis, and/or for which fibrosis is a risk factor. In some embodiments, the disease/disorder associated with fibrosis, or for which fibrosis is a risk factor, is a cancer, e.g. cancer of the liver (e.g. hepatocellular carcinoma).

IL-11/IL-11R signalling is also implicated in the pathology of other diseases/disorders, and the IL-11 binding receptors of the present invention accordingly find use in methods to treat, prevent and/or alleviate the symptoms of these diseases/disorders also.

IL-11/IL-11R signalling has been implicated in the development and progression of various cancers. Studies suggest that IL-11/IL-11R signalling is important for promoting chronic gastric inflammation and associated gastric, colonic, hepatocellular and breast cancer tumorogenesis through excessive activation of STAT3 (Ernst M. et al. J Clin invest. (2008); 118:1727-1738), that IL-11/IL-11R signalling may promote tumorigenesis by triggering the JAK-STAT intracellular signalling pathway, and may also promote metastasis vie signalling through the PI3K-AKT-mTORC1 pathway (Xu at al., Cancer Letters (2016) 373(2) 156-163). Through STAT3, IL-11 promotes survival, proliferation, invasion angiogenesis and metastasis, the IL-11/GP130/JAK/STAT3 signalling axis may be rate-limiting for the progression of gastrointestinal tumors, and elevated IL-11 expression is associated with poor prognosis of breast cancer patients (Johnstone et al., Cytokine & Growth Reviews (2015) 23(5): 489-498). IL-11/IL-11R signalling has also been shown to influence breast cancer stem cell dynamics end tumor heterogeneity (Johnstone et al., Cytokine & Groth Reviews (2015) 26(5): 489-498). Recently IL-11 signalling has been implicated in chemoresistance of lung adenocarcinoma, cancer associated fibroblasts were found to upregulate IL-11, and confer chemoresistance to lung cancer cells through activation of the IL-11/IL-11R/STAT3 anti-apoptotic signalling pathway (Tao et al. 2016, Sci Rep 6:6:38408). IL-11 signalling may promote the fibroblast-to-myofibroblast transition and extracellular matrix production by fibroblasts in the pro-malignant environment (PME) and tumour micro-environment (TME).

In some embodiments, the IL-11 binding receptors of the present invention are provided for use in methods to treat/prevent a cancer. In some embodiments, the cancer may be a cancer which leads directly or indirectly to inflammation and/or fibrosis.

A cancer may be any unwanted cell proliferation (or any disease manifesting itself by unwanted cell proliferation), neoplasm or tumor or increased risk of or predisposition to the unwanted cell proliferation, neoplasm or tumor. The cancer may be benign or malignant and may be primary or secondary (metastatic). A neoplasm or tumor may be any abnormal growth or proliferation of cells and may be located in any tissue.

In some embodiments, the IL-11 binding receptors of the present invention are provided for use in methods to treat/prevent a cancer, e.g. an epithelial cell cancer, breast cancer gastrointestinal cancer (e.g. esophageal cancer, stomach cancer, pancreatic cancer, liver cancer, gallbladder cancer, colorectal cancer, anal cancer, gastrointestinal carcinoid tumor), and lung cancer (e.g. non-small cell lung cancer (NSCLC) or small cell lung cancer (SCLC))). In some embodiments, the cancer is a cancer for which acute and/or chronic inflammation is a risk factor.

In some embodiments, the cancer may be associated with increased IL-11, IL-11Rα and/or gp130 gene or protein expression. For example, cells of the caner may have Increased expression of IL-11, IL-11Ra and/or gp130 as compared to comparable, non-cancerous cells, or may be associated with increased expression of IL-11, IL-11Rα and/or gp130 by other cells (e.g. non-cancerous cells) as compared to the level of expression by comparable cells in the absence of a cancer (e.g. in a healthy control subject). In some embodiments, cells of the cancer may be determined to have an increased level of signalling through ERK and/or STAT3 pathways as compared to comparable non-cancerous cells.

In some embodiments, the cancer may be associated with a mutation in IL-11, IL-11Rα and/or gp130. In some embodiments, such mutation may be associated with increased level of gene or protein expression, or may be associated with an increased level of IL-11/IL-11R signalling relative to the level of expression/signalling observed in the absence of the mutation.

IL-11/IL-11R signalling has also been implicated in diseases/disorder characterised by inflammation. Intra-articulator injection of IL-11 has been shown to cause joint inflammation (Wong et al., Cytokine (2006) 29:72-76), and IL-11 has been shown to be proinflammatory at sites of IL-13-mediated tissue inflammation (Chen et al., J Immunol (2005) 174:2305-2313). IL-11 expression has also been observed to be significantly increased in chronic skin lesions in atopic dermatitis, and Is known to be involved in bronchial Inflammation (Toda et al. J Allergy Clin Immunol (2003) 111:875-881). IL-11/IL-11R signalling is implicated in inflammatory bowel disease (ISO) and asthma (Putoczki and Ernst. J Leuko Biol (2010) 88(6)1109-1117) IL-11 has also been identified as a risk factor for multiple sclerosis; IL-11 is elevated in the cerebrospinal fluid of patients with critically isolated syndrome (CIS) as compared to control subjects, and serum levels of IL-11 are higher during relapses for patients with relapsing-remitting multiple sclerosis, and IL-11 may promote differentiation CD4+ T cells to a T_{H}17 phenotype—T_{H}17 cells are important cells in the pathogenesis of multiple sclerosis (Zhang at al., Oncotarget (2015) 8(32): 32297-32298).

In some embodiments, the IL-11 binding receptors of the present invention are provided for use in methods to treat/prevent a disease/disorder characterised by inflammation. In some embodiments, a disease or disorder characterised by inflammation may be a disease/disorder which loads directly or indirectly to a cancer and/or fibrosis. Diseases characterised by inflammation include e.g. allergic inflammation such as allergic asthma and bronchial inflammation, atopic dermatitis, allergic rhinitis and ocular allergic diseases, and autoimmune diseases such as multiple sclerosis, systemic lupus erythematosus, rheumatoid arthritis, chronic active hepatis, type 1 diabetes mellitus, celiac disease, Grave's disease, uveitis, pemphigus, psoriasis, Crohn's disease, ulcerative colitis, inflammatory bowel disease, anaemia and autoimmune thyroiditis.

in some embodiments, the IL-11 binding receptors of the present invention are provided for use in methods to treat/prevent a disease/disorder associated with infection, in particular where infection leads directly or indirectly to fibrosis, cancer or inflammation. A disease associated with infection may be a disease which is caused or exacerbated by infection with the relevant infectious agent, or may be a disease for which infection with the relevant infectious agent is a risk factor.

An infection may be any infection or infectious disease, e.g. bacterial, viral, fungal, or parasitic infection. In particular embodiments, the disease/disorder may be associated with a viral Infection. In some embodiments it may be particularly desirable to treat chronic/persistent infections, e.g. where such infections are associated with inflammation, cancer and/or fibrosis.

The infection may be chronic, persistent, latent or slow, and may be the result of bacterial, viral, fungal or parasitic infection. As such, treatment may be provided to patients having a bacterial, viral or fungal infection. Examples of bacterial infections include infection with Helicobacter pylori and Mycobacterium tuberculosis infection of the lung. Examples of val infections include infection with EBV, HPV, HIV, hepatitis B or hepatitis C.

The treatment may involve ameliorating, treating, or preventing the disease/disorder by inhibiting the biological activity of IL-11 or an IL-11-containing complex. Such methods may include the administration of the IL-11 binding receptors/compostions according to the present invention to bind to ad inhibit the biological activity of IL-11 or an IL-11-containing complex. Herein, inhibiting the biological activity of IL-11 or an IL-11-containing complex may be referred to as ‘neutralising’.

Methods of treatment may optionally include the co-administration of biological adjuvants (e.g., interleukins, cytokines. Bacillus Comette-Guenrin, monophosphoryl lipid A, etc.) in combination with conventional therapies for treating cancer such as treatment with an agent for treating cancer (e g chemotherapy), radiation, or surgery. Methods of treatment may involve administering a composition according to the present invention as a vaccine that works by activating the immune system to prevent or destroy cancer cell growth. Methods of medical treatment may also involve in vivo, ex vivo, adoptive immunotherapies, including those using autologous and/or heterologous cells or immortalized cell lines.

The treatment may be aimed at prevention of a disease/disorder associated with overactive/elevated IL-11/IL-11R mediated signalling. As such, the IL-11 binding receptors may be used to formulate pharmaceutical compositions or medicaments and subjects may be prophylactically treated against development of a disease state. This may take place before the onset of symptoms of the disease state, and/or may be given to subjects considered to be at greater risk of the disease or disorder.

Treatment may comprise co-therapy with a vaccine, which may involve simultaneous, separate or sequential therapy, or combined administration of vaccine and the IL-11 binding receptor or composition according to the invention.

Administration of receptor is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc. is within the responsibility of general practitioners and other medical doctors, and typically takes account of the disorder to be treated, the condition of the individual patient, the site of delivery, the method of administration and other factors known to practitioners. Example of the techniques and protocols mentioned above can be found in Remington's Pharmaceutical Sciences, 20th Edition, 2000, pub. Lippincott, Williams & Wilkins.

### Formulations Pharmaceutically Useful Compositions and Medicaments

IL-11 binding receptors according to the present invention may be formulated as pharmaceutical compositions or medicaments for clinical use and may comprise a pharmaceutically acceptable carrier, diluent, excipient or adjuvant.

The composition may be formulated for topical, parenteral, systemic, intracavitary, intravenous, intra-arterial, intramuscular, intrathecal, intraocular, intraconjunctival, intratumoral, subcutaneous, oral or transdermal routes of administration which may include injection or infusion. Suitable formulations may comprise the IL-11 binding receptor in a sterile or isotonic medium. Medicaments and pharmaceutical compostions may be formulated in fluid, including gel, form. Fluid formulations may be formulated for administration by injection or via catheter to a selected region of the human or animal body.

In accordance with the present invention methods are also provided for the production of pharmaceutically useful compositions, such methods of production may comprise one or more steps selected from: isolating an IL-11 binding receptor as described herein; and/or mixing an isolated receptor as described herein with a pharmaceutically acceptable carrier, adjuvant, excipient or diluent.

For example, a further aspect of the present invention relates to a method of formulating or producing a medicament or pharmaceutical composition for use in a method of medical treatment, the method comprising formulating a pharmaceutical composition or medicament by mixing an IL-11 binding receptor as described herein with a pharmaceutically acceptable carrier, adjuvant, excipient or diluent.

### Methods of Detection

IL-11 binding receptors described herein may be used in methods that involve the binding d the IL-11 binding receptor to IL-11 Such methods may Involve detection of the bound complex of receptor and IL-11. As such, in one embodiment a method is provided, the method comprising contacting a sample containing, or suspected to contain, IL-11 with an IL-11 binding receptor as described herein and detecting the formation of a complex of the IL-11 binding receptor, and IL-11.

Suitable method formats are well known in the art, including immunoassays such as sandwich assays e.g. ELISA. The method may Involve labelling the IL-11 binding receptor, or IL-11, or both, with a detectable label, e.g. fluorescent, luminescent or radio-label. IL-11 expression may be measured by immunohistochemistry (IHC), for example of a tissue sample obtained by biopsy. In some embodiments, the label may be selected from: a radio-nucleotide, positron-emitting radionuclide (e.g. for positron emission tomography (PET)), MRI contrast agent or fluorescent label.

Analysis m vitro or in vivo of processes mediated by IL-11 may involve analysis by positron emission tomography (PET), magnetic resonance imaging (MRI), or fluorescence imaging, e.g. by detection of appropriately labelled species.

Methods of this kind may provide the basis of a method of diagnosis of a disease or condition requiring detection and or quantitation of IL-11 or an IL-11-containing complex. Such methods may be performed in vitro on a subject sample, or following processing of a subject sample. Once the sample is collected, the subject as rot required to be present for the in vitro method of diagnosis to be performed and therefore the method may be one which is not practised on the human or animal body.

Such methods may involve determining the amount of IL-11 or IL-1l-containing complex present in a subject sample. The method may further comprise comparing the determined amount against a standard or reference value as part of the process of reaching a diagnosis. Other diagnostic tests may be used in conjunction with those described here to enhance the accuracy of the diagnosis or prognosis or to confirm a result obtained by using the tests described here.

The level of IL-11 or IL-11-containing complex present in a subject sample may be indicative that a subject may respond to treatment with an IL-11 binding receptor, e.g. an IL-11 binding receptor or composition according to the present invention. The presence of a high level of IL-11 or IL-11-containing complex in a sample may be used to select a subject for treatment with an IL-11 binding receptor or composition described herein. The IL-11 binding receptors of the present invention may therefore be used to select a subject for treatment with IL-11 binding receptor therapy.

Detection in a sample of IL-11 or IL-11-containing complex may be used for the purpose of diagnosis of an infectious disease, autoimmune disorder or a cancerous condition in the subject, diagnosis of a predisposition to an infectious disease, autoimmune disorder or a cancerous condition or for providing a prognosis (prognosticating) of an infectious disease, autoimmune disorder or a cancerous condition. The diagnosis or prognosis may relate to an existing (previously diagnosed) infectious, inflammatory or autoimmune disease/disorder or cancerous condition.

A sample may be taken from any tissue or bodily fluid. The sample may comprise or may be derived from: a quantity of blood; a quantity at serum derived from the individual's blood which may comprise the fluid portion of the blood obtained after removal of the fibrin clot and blood cells; a tissue sample or biopsy; pleural fluid; cerebrospinal fluid (CSF); or cells isolated from said individual. In some embodiments, the sample may be obtained or derived from a issue or tissues which are affected by the disease/disorder (e.g. tissue or tissues in which symptoms of the disease manifest, or which are involved in the pathogenesis of the disease/disorder).

Methods according to the present invention may preferably be performed in vitro. The term “in vitro” as intended to encompass experiments with cells in culture whereas the term “in vivo” is intended to encompass experiments with and/or treatment of intact multi-cellular organisms.

### Combination Therapies

IL-11 binding receptors and compositions according to the present invention may be administered alone or in combination with other treatments. Administration of such combination may be simultaneous or sequential, depending on the disease/disorder to be treated. The other treatment with which the IL-11 binding receptor or composition is administered may be aimed at treating or preventing the disease/disorder. In some embodiments, the other treatment with which the IL-11 binding receptor or composition is administered may be aimed at treating or preventing e.g. infection, inflammation and/or cancer.

Simultaneous administration refers to administration of the IL-11 binding receptor and therapeutic agent together, for example as a pharmaceutical composition containing both agents (combined preparation), or immediately after each other and optionally via the same route d administration, e.g. to the same artery, vein or other blood vessel.

Sequential administration refers to administration of one of the IL-11 binding receptor or therapeutic agent followed after a given time interval by separate administration of the other agent, it is not required that the two agents are administered by the some route, although this is the case in some embodiments. The time interval may be any time interval.

In some embodiments, treatment with an IL-11 binding receptor or composition of the present invention may be accompanied by an agent for treating or preventing infection (e.g. an antibiotic, anti-viral, anti-fungal or anti-parasitic agent). In some embodiments, treatment with an IL-11 binding receptor or composition of the present invention may be accompanied by an agent for treating or preventing inflammation (e.g. a non-steroidal anti-inflammatory drug (NSAID). In some embodiments, treatment with an IL-11 binding receptor or composition of the present invention may be accompanied by radiotherapy (i.e. treatment with ionising radiation, e.g. X-rays or γ-rays) and/or an agent for treating or preventing cancer (e.g. a chemotherapeutic agent). In some embodiments, the IL-11 binding receptor composition of the present Invention may be administered as part of a combination treatment with an immunotherapy.

A treatment may involve administration of more then one drug. A drug may be administered alone or in commination with other treatments, either simultaneously or sequentially dependent upon the condition to be treated.

### Routes of Administration

IL-11 binding receptors, medicaments and pharmaceutical compositions according to aspects of the present invention may be formulated for administration by a number of routes, including but not limited to, parenteral, intravenous, intra-arterial, intraocular, intraconjunctival, intramuscular, subcutaneous, intradermal, intratumoral injection or infusion, and oral administration. IL-11 binding receptors, polypeptides and other therapeutic agents, may be formulated in fluid or solid form. Fluid formulations may be formulated for administration by injection or infusion b a selected region of the human or animal body.

### Kits

In some aspects of the present invention a kit of parts is provided. In some embodiments the kit may have at least one container having a predetermined quantity of the IL-11 binding receptor or composition. The kit may provide the IL-11 binding receptor in the form of a medicament or pharmaceutical composition, and may be provided together with instructions for administration to a subject in order to treat a specified disease/disorder. The IL-11 binding receptor or composition may be formulated so as to be suitable for injection or infusion to a tumor or to the blood.

In some embodiments the kit may further comprise at least one container having a predetermined quantity of another therapeutic agent (e.g. anti-infective agent or chemotherapy agent). In such embodiments, the kit may also comprise a second medicament or pharmaceutical composition such that the two medicaments or pharmaceutical compositions may be administered simultaneously or separately such that they provide a combined treatment for the specific disease or condition. The therapeutic agent may also be formulated so as to be suitable for injection or infusion to a tumor or to the blood.

### Subjects

The subject to be treated may be any animal or human. The subject is preferably mammalian, more preferably human. The subject may be a non-human mammal, but is more preferably human. The subject may be male or female. The subject may be a patient. A subject may have been diagnosed with a disease or condition requiring treatment, or be suspected of having such a disease or condition.

In some embodiments the subject may be at risk of developing/contracting a disease or disorder.

### Protein Expression

Molecular biology techniques suitable for producing the proteins (e.g. the IL-11 binding receptors) according to the invention in cols are we known in the art, such as those set out in Sambrook et al. Molecular Cloning: A Laboratory Manual, New York: Cold Spring Harbor Press, 1989

The polypeptide may be expressed from a nucleotide sequence. The nucleotide sequence may be contained in a vector present in a cell, or may be incorporated into the genome of td WI.

A “vector” as used herein is an oligonucleotide molecule (DNA or RNA) used as a vehicle to transfer exogenous genetic material into a cell. The vector may be an expression vector for expression of the genetic material in the cell. Such vectors may include a promoter sequence operably linked to the nucleotide sequence encoding the gene sequence to be expressed. A vector may also include a termination codon and expression enhancers. Any suitable vectors, promoters, enhancers and termination codons known in the art may be used to express polypeptides from a vector according to the invention. Suitable vectors include plasmids, binary vectors, viral vectors and artificial chromosomes (e.g. yeast artificial chromosome).

In this specification the term ‘operably linked’ may Include the situation where a selected nucleotide sequence and regulatory nucleotide sequence (e.g. promoter nd/or enhancer) are covalently linked in such a way as to place the expression of the nucleotide sequence under the influence or control of the regulatory sequence (thereby forming an expression cassette). Thus a regulatory sequence is operably linked to the selected nucleotide sequence if the regulatory sequence is capable of effecting transcription of the nucleotide sequence. Where appropriate, the resulting transcript may then be translated into a desired protein or polypeptide.

Any cell suitable for the expression of polypeptides may be used for producing polypeptides according to the invention. The cell may be a prokaryote or eukaryote. Suitable prokaryotic cells include E. coli. Examples of eukaryotic cells include a yeast cell, a plant cell, insect cell or a mammalian cell (e.g. Chinese Hamster Ovary (CHO) cells). In some cases the cell is not a prokaryotic cot because some prokaryotic cells do not allow for the same post-translational modifications as eukaryotes. In addition, very high expression levels are possible in eukaryotes end proteins can be easier to purify from eukaryotes using appropriate togs. Specific plasmids may also be utilised which enhance secretion of the protein into the media.

Methods of producing a polypeptide of interest may involve culture or fermentation of a cell modified to express the polypeptide. The culture or fermentation may be performed in a bioreactor provided with an appropriate supply of nutrients, air/oxygen and/or growth factors. Secreted proteins ca be collected by partitioning culture media/fermentation broth from the cells, extracting the protein content, and separating individual proteins to isolate secreted polypeptide. Culture, fermentation and separation techniques are well known to those of skill in the art.

Bioreactors Include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culture of cells that express the polypeptide of interest, that polypeptide is preferably isolated. Any suitable method for separating polypeptides from cell culture known in the art may be used. In order to isolate a polypeptide of interest from a culture, it may be necessary to first separate the cultured cells from media containing the polypeptide of interest. If the polypeptide of interest is secreted from the cells, the cells may be separated from the culture media that contains the secreted polypeptide by centrifugation. If the polypeptide of interest collects within the cell, it will be necessary to disrupt the cells prior to centrifugation, tar example using sonification, rapid freeze-thaw or osmotic lysis. Centrifugation will produce a pellet containing the cultured cells, or cell debris of the cultured cells, and a supernatant containing culture medium and the polypeptide of interest.

It may then be desirable to isolate the polypeptide of interest from the supernatant or culture medium, which may contain other protein and non-protein components. A common approach to separating polypeptide components from a supernatant or culture medium is by precipitation. Polypeptides/proteins of different solubility are precipitated at different concentrations of precipitating agent such as ammonium sulfate. For example, at low concentrations of precipitating agent, water soluble proteins are extracted. Thus, by adding increasing concentrations of precipitating agent proteins of different solubility may be distinguished. Dialysis may be subsequently used to remove ammonium sulfate from the separated proteins.

Other methods for distinguishing different polypeptides/proteins are known in the art, for example ion exchange chromatography and size chromatography. These may be used as an alternative to precipitation, or may be performed subsequently to precipitation.

Once the polypeptide of interest has been isolated from culture it may be necessary to concentrate the protein. A number of methods for concentrating a protein of interest are known in the art, such as ultrafiltration or lyophilisation.

### Sequence Identity

Alignment for purposes of determining percent amino acid or nucleotide sequence identity can be achieved in various ways known to a person of skill in the art, for instance, using publicly available computer software such as CustalW 1.82. T-coffee or Megalign (DNASTAR) software. When using such software, the default parameters, e.g. for gap penalty and extension penalty, are preferably used. The default parameters of ClustalW 1.82 are: Protein Gap Open Penalty=10.0, Protein Gap Extension Penalty=0.2, Protein matrix Gonnet, Protein/DNA ENDGAP=−1, Protein/DNA GAPDIST=4.

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises,” and “comprising,” will be understood to imply the inclusion of a stated integer or step or group of integers or slope but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment incudes from the one particular value and/or to the other particular value. Similarly, when values are expressed as approximations, by the use of the antecedent “about,” it will be understood that the particular value forms another embodiment.

### BRIEF DESCRIPTION OF THE FIGURES

Embodiments and experiments illustrating the principles of the invention will now be discussed with reference to the accompanying figures, in which:

FIG. 1. Graph showing read depth for whole transcriptome sequencing of human atrial fibroblasts from 160 individuals with and without stimulation with TGFβ1.

FIGS. 2A-2E. Graphs showing expression of endothelial, cardiomyocyte and fibroblast marker genes as determined by RNA seg of the tissue of origin (human atrial tissues samples, n=8) and primary, unstimulated fibroblast cultures. (FIG. 2A) PECAM1, (FIG. 2B) MYH6 (FIG. 2C) TNNT2 (FIG. 2D) COL1A2, and (FIG. 2E) ACTA2.

FIG. 3A-3E. Graphs showing upregulation of IL-11 expression in fibroblasts in response to stimulation with TGFβ1. (FIG. 3A and FIG. 38) Graphs snowing fold change in gene expression in fibrosis; IL-11 is the most upregulated gene in response to TGFβ1 treatment. (FIG. 3C) IL-11 secretion by fibroblasts in response to stimulation with TGFβ1. (FIG. 30) Comparison of IL-11 gene expression in tissues of healthy individuals and in atrial fibroblasts, with or without TGFβ1 stimulation. (FIG. 3E) Correspondence of fold change in IL-11 expression ats determined by RNA-seq vs. qPCR.

FIGS. 4A-4D. Graphs showing induction of IL-11 secretion in primary fibroblasts by various profibrotic cytokines, as determined by ELISA. (FIG. 4A) TGFβ1, ET-1, AngII, PDGF, OSM and IL-13 induce IL-11 secretion, and IL-11 also induces IL-11 expression in a positive feedback loop (FIG. 4B) Graph showing that the ELISA only detects native IL-11 secreted from cells, and does not detect recombinant IL-11 used for the IL-11 stimulation condition. (FIG. 4C) and (FIG. 4D) Cells were stimulated with recombinant IL-11, IL-11 RNA was measured and the native IL-11 protein level was measured in the cell culture supernatant by ELISA at the indicated time points.

FIGS. 5A-5F. Graphs and images showring myofibroblast generation from, and production of ECM and cytokine expression by, atrial fibroblasts in response to stimulation with TGFβ1 or IL-11. (FIG. 5A) myofibroblast generation and ECM production by primary atrial fibroblasts following stimulation with TGFβ1 or IL-11, as measured by fluorescence microscopy following staining for a α-SMA, collagen or periostin. (FIG. 5B) Collagen content of cell culture supernatant as determined by Sirius Red staining. Secretion of the fibrosis markers (FIG. 5C) IL-6, (FIG. 5D) TIMP1 and (FIG. 5E) MMP2 as measured by ELISA (FIG. 5F) Activation of murine fibroblasts by stimulation with human or mouse recombinant IL-11. *P<0.05, **P<0.01, ***P<0.01, ****P<0.0001 [Mean±SD. Dunnett].

FIGS. 6A-6C. Graphs chowing the profibrotic effect of IL-11 (FIG. 6A) Mouse fibroblasts from different tissues of origin can be activated by IL-11 and display increased EGM production. (Mean±SD, Dunnett). Injection of mice with recombinant IL-11 or AngII results in (FIG. 6B) an increase in organ weight [Mean±SEM], and (FIG. 6C) an increase in collagen content (as determined by HPA assay).*P<0.05, **P<0.01, ***P<0.001, ****P<0.0001 [Mean±SD, Dunnett].

FIGS. 7A-7F. Graphs and images showing that IL-11 is required the pro-fibrotic effects of TGFβ1 on fibroblasts. (FIG. 7A) myofibroblast generation and EGM production by primary atrial fibroblasts, with or without stimulation with TGFβ1, and in the presence/absence of neutralising anti-IL-11 antibody or isotype control IgG, as measured by fluorescence microscopy following staining for (FIG. 7A) α-SMA, (FIG. 7B) EdU or (FIG. 7C) Periostin. FIG. 7D to FIG. 7F) Secretion of the fibrosis markers (FIG. 7D) IL-6, (FIG. 7E) TIMP1, and (FIG. 7F) MMP2 was analysed by ELISA. Fluorescence was normalized to the control group without stimulation. [Mean±SD, Dunnett] *P<0.05, **P<0.01, ***P<0.001 or ****P<0.0001.

FIGS. 8A-8B. Graphs and images showing the effect of neutralisation of IL-11 on collagen production triggered by TGFβ1. Collagen production by cardiac fibroblasts with or without stimulation with TGFβ1, and in the presence/absence of neutralising anti-IL-11 antibody or isotype control IgG, as determined by (FIG. 8A) Operetta assay or (FIG. 8B) Sirius Red staining. [Mean±SD, Dunnett] *P<0.05, **P<0.01, ***P<0.001 or ****P<0.0001.

FIG. 9. Graphs showing the ability of various IL-11 and IL-11Rα antagonists to inhibit fibrosis. Human atrial fibroblasts were treated with neutralizing antibody against IL-11, neutralizing antibody against IL-11Rα, siRNA that downregulates IL-11 expression or siRNA that downregulates IL-11 RA expression and the effect on the TGFβ1-driven pro-fibrotic response in fibroblasts in vitro was analysed. [Mean±S.D. Dunnett] *P<0.05, **P<0.01, ***P<0.001 or****P<0.0001.

FIGS. 10A-10D. Bar charts showing the response of fibroblasts from IL-11-RA knockout mine to pro-fibrotic treatment. Fibroblasts derived from IL-11RA WT (+/−). Heterozygous (+/−) and Homozygous null (−/−) mice were incubated for 24 h with TGFβ1, IL-11 or AngII (5 ng/ml). (FIG. 10A) Percentage of myofibroblasts as determined by analysis αSMA content. (FIG. 10B) Percentage proliferating cells as determined by staining for EdU, (FIG. 10C) Collagen content and (FIG. 10D) ECM production as measured by detection of periostin [Mean±SD].

FIGS. 11A-11B. Graphs showing the effect of IL-11 neutralisation on fibrosis in response to various pro-fibrotic stimuli. Fibroblasts were cultured in vitro in the presence/absence of various different pro-fibrotic factors, and in the presence/absence of neutralising anti-IL-11 antibody or pan anti-TGFβ antibody (FIG. 11A) Collagen production and (FIG. 11B) myofibroblast generation as determined by analysis of αSMA expression. [Mean±SD, Dunnett] *P<0.05, **P<0.01, ***P<0.001 or ****P<0.0001,

FIGS. 12A-12D. Bar charts showing expression of markers of fibrosis in the atrium and heart of WT and IL-11RA (−/−) animals following treatment with AngII treatment. (FIG. 12A) Collagen content, as measured by hydroxyproline assay. (FIG. 12B) Collagen (Col1A2) expression. (FIG. 12C) αSMA (ACTA2) expression. (FIG. 12D) Fibronectin (Fn1) expression.

FIGS. 13A-13C. Schematics of the experimental procedures for analysing fibrosis in (FIG. 13A) lung, (FIG. 13B) skin and (FIG. 13C) eye for IL-11RA−/− mice as compared to IL-11RA+/+ mice.

FIGS. 14A-14B. Scatterplots showing fold change in gene expression. (FIG. 14A) Fold changes in gene expression in fibroblasts following stimulation with TGFβ1, IL-11 or TGFβ1 and IL-11. (FIG. 14B) Fold changes in gene expression m fibroblasts obtained from IL-11RA+/+ and IL-11RA−/− mice following stimulation with TGFβ1.

FIG. 15. Graphs showing the effect of IL-11RA knockout on folate-induced kidney fibrosis as measured by collagen content in kidney tissue.

FIGS. 16A-16B. Photographs showing the effect of IL-11RA knockout on wound healing and fibrosis in the eye following trabeculectomy (filtration surgery). (FIG. 16A) Eye sections of IL-11RA+/+ (WT) and IL-11RA−/− (KO) animals 7 days after filtration surgery. (FIG. 16B) Maturation of collagen fibres as evaluated by picro-sirius red/polarization light technique (Szendröl et al. 1984, Acta Morphol Hung 32, 47-55) more fibrosis is observed in WT mice than KO mice.

FIGS. 17A-17B. Amino acid sequences of decoy IL-11 receptors, and component sequences thereof. (FIG. 17A) Amino acid sequences for Decoy IL-11 Receptor 1 (D11R1). (FIG. 17B) Amino acid sequences for Decoy IL-11 Receptor 2 (D11R2).

FIGS. 18A-18D. Nucleotide Sequencer of vector inserts encoding decoy IL-11 receptors, and component sequences thereof. EcoRI, Kozak and HindIII sequences are indicated (underlined). (FIG. 18A) Nucleotide sequences for Decoy IL-11 Receptor 1 (D11R1). (FIG. 18B) Nucleotide sequences for Decoy IL-11 Receptor 2 (D11R2).

FIGS. 19A-19B Graphs showing the effect of decoy IL-11 receptors on fibrosis in response to stimulation with TGFβ1. Fibroblasts were cultured in vitrain the presence/absence of TGFβ1 (5 ng/ml), in the presence or absence of (FIG. 19A) D11R1 (Decoy Receptor 50aa Linker) or (FIG. 19B) D11R2 (Decoy Receptor 33aa Linker), at various different concentrations. Myofibroblast generation after 24 hours (i.e. the percentage of activated fibroblasts) was determined by analysis of αSMA expression.

FIGS. 20A-20B. Amino acid sequence alignment of human and murine gp130 sequences. (FIG. 20A) alignment of extracellular domain (ECD) regions of human and murine gp130. (FIG. 20B) alignment of cytokine binding modules (i.e. D1 to D3) of human and murine gp130.

FIGS. 21A-21B. Amino acid sequence alignment of human and murine IL-11Rα sequences. (FIG. 21A) alignment of extracellular domain (ECD) regions of human and murine IL-11Rα. (FIG. 21B) alignment of cytokine binding modules (i.e. D2 and D3) of human and murine IL-11Rα.

FIGS. 22A-22D. Graphs showing that IL-11 is required for the pro-fibrotic effects of TGFβ1 in liver fibroblasts. Activation and proliferation of primary human liver fibroblasts with or without stimulation with TGFβ1, and in the presence/absence of neutralising anti-IL-11 antibody or isotype control IgG, as measured by analysis of the proportion of (FIG. 22A) α-SMA positive cells, (FIG. 22B) EdU positive cells, (FIG. 22C) Collagen positive cells and (FIG. 22D) Periostin positive cells as compared to the unstimulated cells (Baseline). [Mean±SD, Dunnett] *P<0.05, **P<0.01, ***P<0.001 or ****P<0.0001.

FIG. 23. Bar chart showing that IL-11 is required for the pro-fibrotic effects of TGFβ1 in skin fibroblasts. Activation of mouse skin fibroblasts, with or without stimulation with TGFβ1, and in the presence/absence of neutralising anti-IL-11 antibody, as measured by analysis of the percentage of α-SMA positive cells (activated fibroblasts).

FIG. 24. Bar chart showing lung fibroblast ceil migration with and without IL-11 signalling. Migration of lung fibroblasts from IL-11RA+/+ (WT) and IL-11 RA−/− (KO) animals was analysed in an in vitro scratch assay without stimulus, or in the presence of TGFβ1 or IL-11.

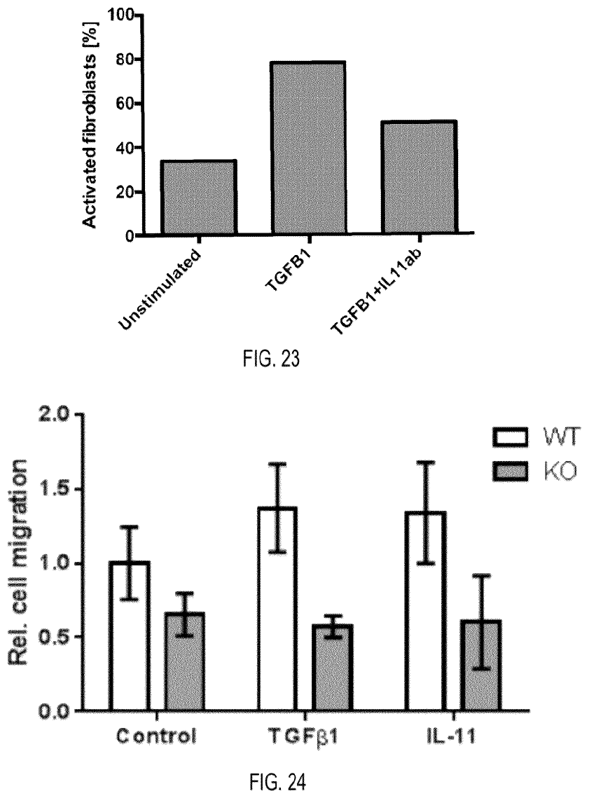

FIGS. 23A-25B. Graphs showing fibroblast activation in response to hyper IL-11. Cells were stimulated with the indicated amount (in ng/ml) of hyper IL-11 or recombinant IL-11, and fibroblast activation was measured by analysis of the percentage of α-SMA positive cells. (FIG. 25A) and (FIG. 25B) present the results of two different experiments.

FIG. 26. Graph showing induction of IL-11 secretion in primary fibroblasts by hyper IL-11. Cells were stimulated with hyper IL-11, and IL-11 RNA and native IL-11 protein levels were measured in the cell culture supernatant by ELISA at the indicated time points.

FIGS. 27A-27B. Graphs showing relative Inhibition of IL-11 mediated signalling by decoy IL-11 receptors as compared to anti-IL-11 antibody. Activation of human atrial fibroblasts, with or without stimulation with TGFβ1, and in the presence of anti-IL-11 antibody. D11R1 (FP50), D11R2 (FP33), or a multi-tag fusion protein control. Fibroblast activation was analysed by determining the percentage of αSMA positive cells. (FIG. 27A) and (FIG. 27B) present the results of two different experiments.

FIG. 28. Graphs showing relative inhibition of IL-11 mediated signalling by decoy IL-11 receptors as compared to anti-IL-11 antibody. Activation of human atrial fibroblasts, with or without stimulation with TGFβ1, and in the presence of 2 μg/ml anti-IL-11 antibody, 500 ng/ml D11R1 (FP50) or 500 ng/ml D11R2 (FP33). Fibroblast activation was analysed by determining the percentage of αSMA positive cells. [Mean±SD, Dunnett] ***P<0.0001.

### EXAMPLES

In the following Examples, the inventors identify a role for IL-11/IL-11R signalling in fibrosis in a variety of tissues, and describe the design, production and functional characterisation of decoy IL-11 receptors.

### Example 1: a Role for IL-11 in Fibrosis

### 1.1 IL-11 is Upregulated in Fibrosis

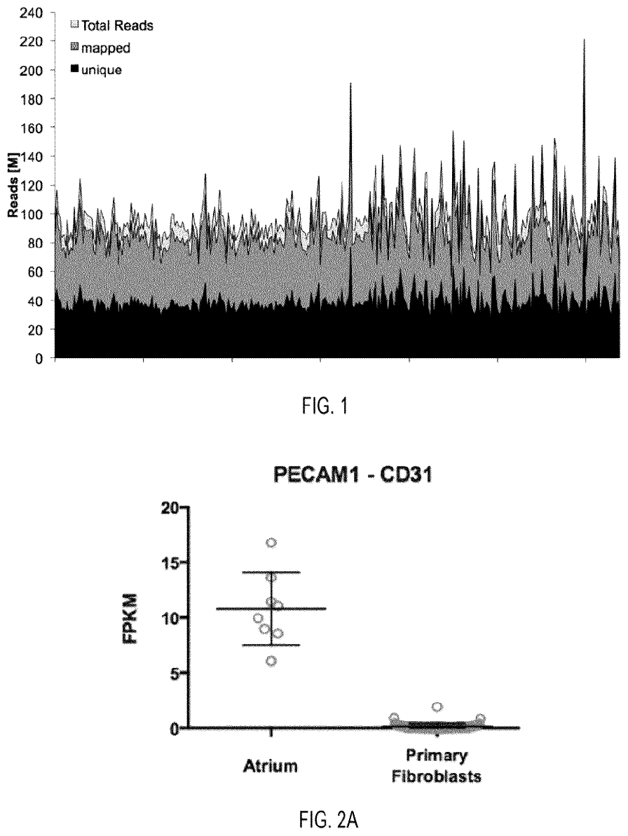

To understand the molecular processes underlying the transition of fibroblasts to activated myofibroblasts, atrial tissue was obtained from more than 200 patients that underwent cardiac bypass surgery at the National Heart Centre Singapore. Cells were cultured in vitro at low passage (passage <4), and either not stimulated or stimulated with TGFβ1 for 24h. We subsequently performed high-throughput RNA sequencing (RNA-seq) analysis of unstimulated fibroblasts and cells stimulated with the prototypic pro-fibrotic stimulus TGFβ1 across 160 individuals; average reed depth was −70M reads per sample (paired-end 100 bp; FIG. 1).

To ensure the purity of the atrial fibroblast cell cultures, we analysed expression of endothelial cell, cardiomyocyte and fibroblast cell type marker genes from the atrium (Hsu et al., 2012 Circulation Cardiovasc Genetics 5, 327-335) in the RNA-seq dataset.

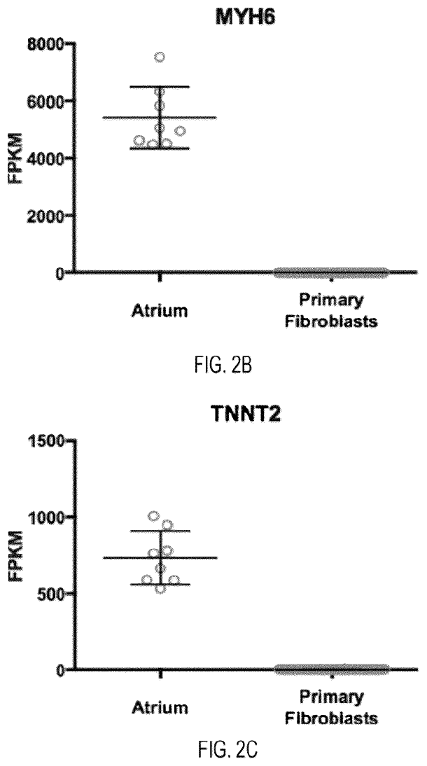

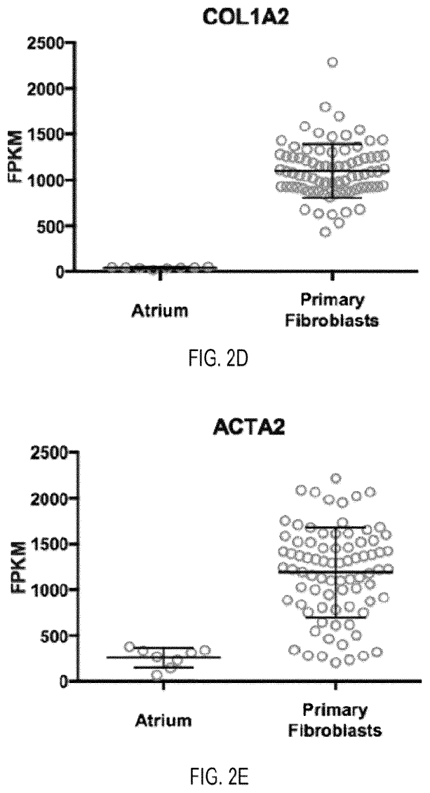

The results are shown in FIGS. 2A to 2E, and confirm the purity of the atrial fibroblast cultures.

Gene expression was assessed by RNA-seq of the tissue of origin (human atrial tissues samples, n=8) and primary, unstimulated fibroblast cultures. No/very ow expression of the endothelial cell marker PECAM1 (FIG. 2A), and the cardiomyocyte markers MYH6 (FIG. 2B) and TNNT2 (FIG. 2C) was detected in the fibroblast cell culture samples. Markers for fibroblasts COL1A2 (FIG. 2D) and ACTA2 (FIG. 2E) were highly expressed compared to the tissue of origin.

Next, the RNA-se data was analysed to identify genes whose expression was increased or decreased upon stimulation with TGFβ1, and this information was integrated with the large RNA-seq dataset across 35+ human tissues provided by the GTEx project (The GTEx Consortium, 2015 Science 348, 648-660). This enabled the identification of gene expression signatures that were specific to the fibroblast-myofibroblast transition.

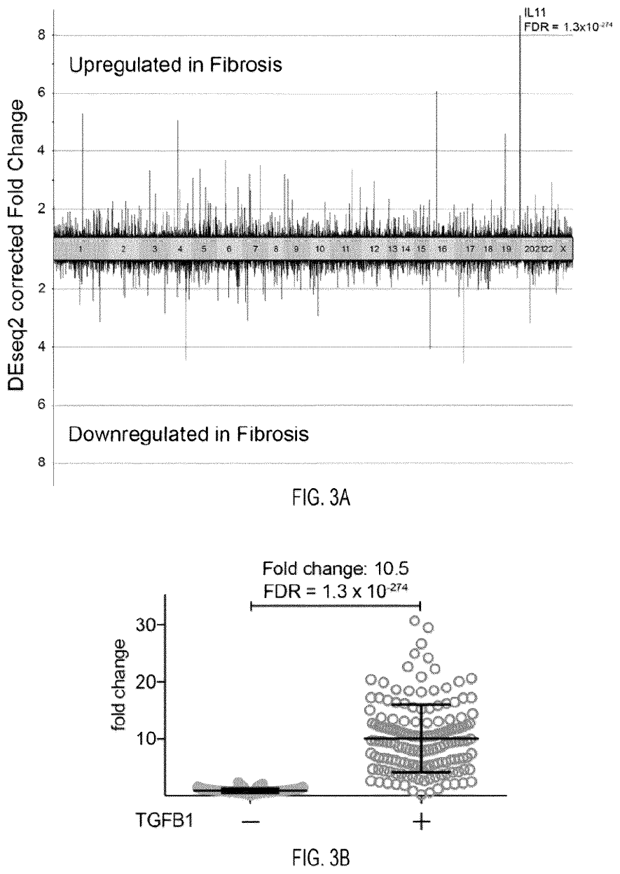

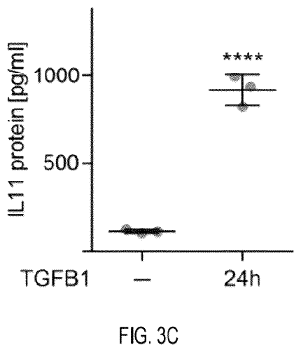

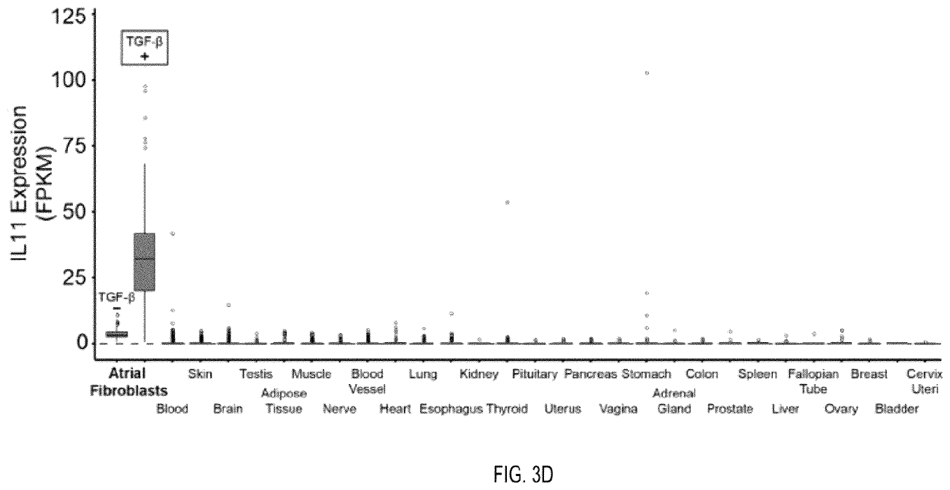

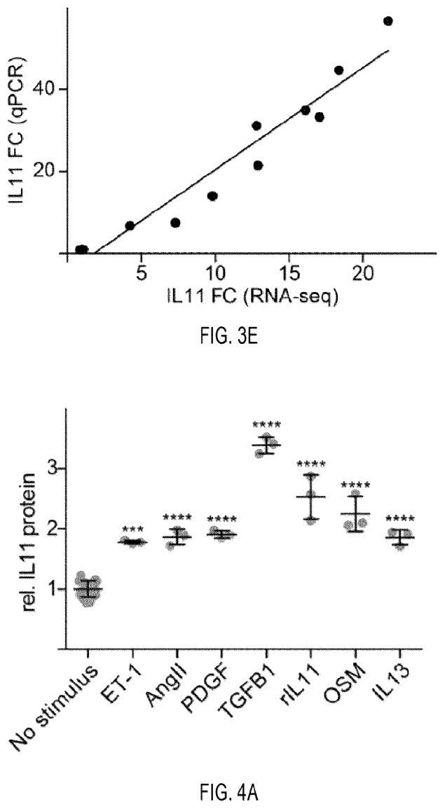

The results are shown in FIGS. 3A to 3E. Across the 10000+ genes expressed in the fibroblasts, IL-11 was the most strongly upregulated gene in response to stimulation with TGFβ1, od on average across the 160 individuals was upregulated more than 10-fold (FIG. 3B).

Upregulation of IL-11 expression was confirmed by ELISA analysis of the cell culture supernatant of TGFβ1 stimulated fibroblasts (FIG. 3C). As compared to the level of expression level of IL-11 in other tissues of healthy individuals, this response was observed to be highly specific to activated fibroblasts (FIG. 3D). Various fold changes of IL-11 RNA expression were also confirmed by qPCR analysis (FIG. 3E).

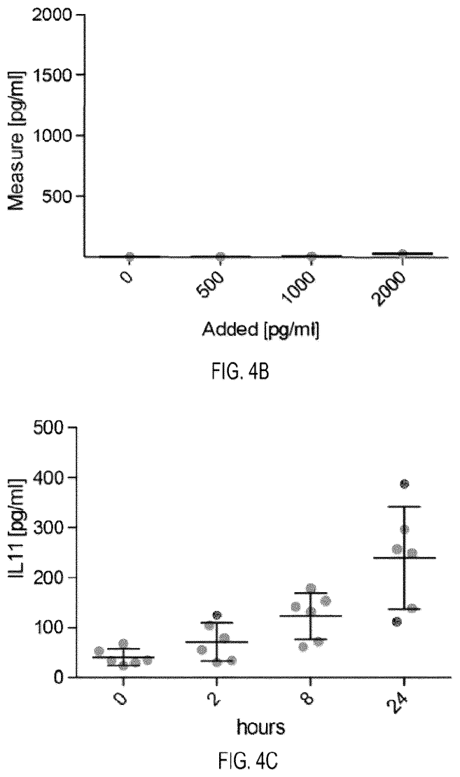

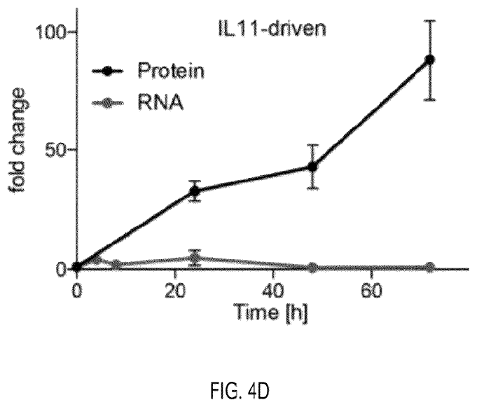

Next, fibroblasts were cultured in vitro and stimulated with several other known pro-fibrotic factors: ET-1, ANGII, PDGF, OSM and IL-13, and also with human recombinant IL-11. For analysing upregulation of IL-11 produced in response to stimulation with IL-11, it was confirmed that the ELISA was only able to detect native IL-11 secreted from cells and does not detect recombinant IL-11 used for the stimulations (FIG. 4B).

The results are shown in FIG. 4A. Each factor was found to significantly induce IL-11 secretion from fibroblasts, IL-11 Is shown to act in an autocrine loop in fibroblasts, which can result in an upregulation of IL-11 protein as much as 100-fold after 72 hours (FIG. 4D).

Interestingly, this autocrine loop for IL-11 is similar to the autocrine production of IL-6. IL-6 is from the same cytokine family and also signals via the gp130 receptor (Garbers and Scheler. 2013 Biol Chem 394, 1145-1161), which is proposed to ensure the continued survival and growth of lung and breast cancer cells (Grivennikov and Karin, 2006 Cancer Cel 13, 7-9).

No increase in IL-11 RNA level was detected in response to stimulation with IL-11 (FIG. 4D). Unlike TGFβ1, which, increases IL-11 expression at both the RNA and protein level, therefore IL-11 seems to upregulate IL-11 expression only at the post-transcriptional level.

### 1.2 IL-11 has a Profibrotic Role in Fibrosis of Heart Tissue

To explore whether the autocrine production of IL-11 is pro- or anti-fibrotic, fibroblasts were cultured in vitro with recombinant IL-11, and the fraction of myofibroblasts (αSMA-positive cells) and extracellular matrix production was analysed.

The expression of αSMA, collagen and periostin was monitored with the Operetta High-Content Imaging System in an automated, high-throughput fashion. In parallel secretion of fibrosis marker proteins such as MMP2, TIMP1 and IL-6 was analysed by ELISA assays, and the levels of collagen were confirmed by calorimetric Sirius Red analysis d the cell culture supernatant.

Briefly, arial fibroblasts derived from 3 Individuals were incubated in 2 wells each for 24n without stimulation, with TGFβ1 (5 ng/ml), or with IL-11 (5 ng/ml). Following incubation, cells were stained to analyse α-SMA content to estimate the fraction of myofibroblasts, and for collagen and periostin to estimate ECM production Fluorescence was measured in 7 fields per well. The supernatant of 2 wells per individual was also assessed for collagen content ty Sirius Red staining. The signal was normalized to the control group without stimulation Secretion of the fibrosis markers IL-6, TIMP1 and MMP2 was analysed via ELISA.

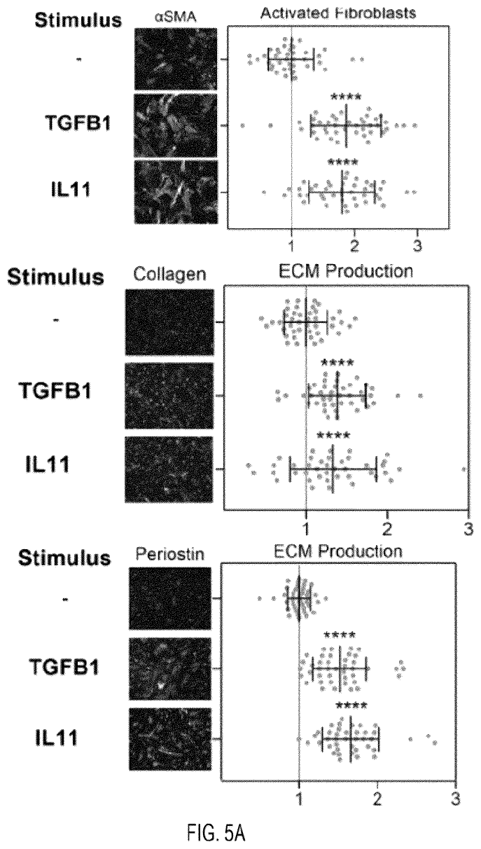

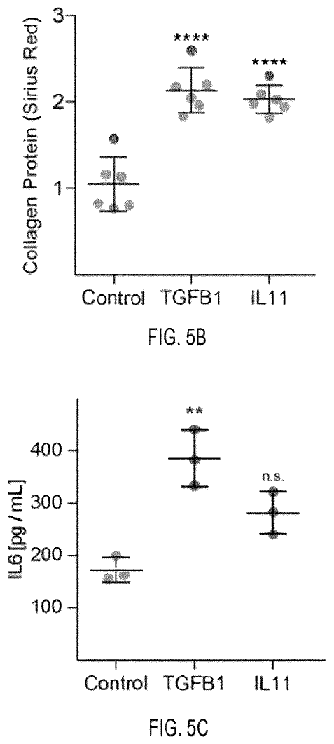

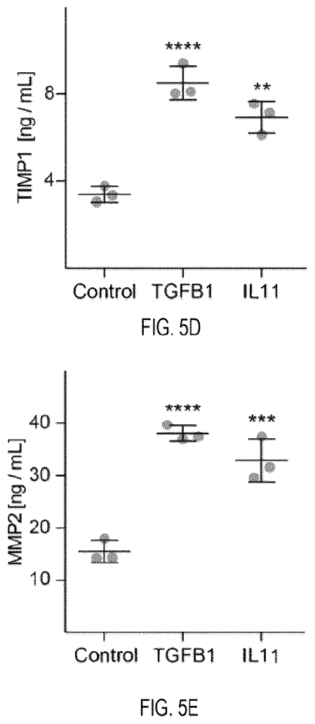

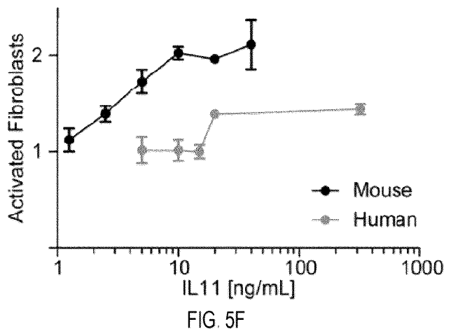

The results are shown in FIGS. 5A to 5F. TGFβ1 activated fibroblasts and increased ECM production (FIG. 5A). Unexpectedly, and in contrast with the anti-fibrotic role described for IL-11 in heart tissue in the scientific literature, recombinant IL-11 caused an increase in the fraction of myofibroblasts in fibroblast cultures, and also promoted the production of extracellular matrix proteins collagen and periostin to the same extent as TGFβ1 (FIG. 5A). Both of IL-11 and TGFβ1 cytokines also significantly increased the secretion of pro-fibrotic markers IL-6, TIMP1 and MMP2 (FIGS. 5B to 5E), and to a similar level.

The inventors hypothesized that the contradiction between the present finding that IL-11 is profibrotic in heart tissue and the antifibrotic role described in the literature might be related to the use of human IL-11 in rodents those previous studies (Obana et al., 2010, 2012; Stangou et al., 2011; Trepicchio and Dorner, 1998).

To investigate this hypothesis, serial dilutions of both human and mouse IL-11 were performed, and the activation of human arial fibroblasts was monitored (FIG. 5F). No activation of fibroblasts was observed at low concentrations of human IL-11 on mouse cells, suggesting that previous Insights into IL-11 function may in part be due to IL-11-non-specific observations.

### 1.3 IL-11 has a Profibrotic Role in Fibrosis of a Variety of Tissues

To test whether the profibrotic action of IL-11 was specific to atrial fibroblasts, human fibroblasts derived from several different tissues (heart, lung, skin, kidney and liver) were cultured in vitro, stimulated with human IL-11, and fibroblast activation and ECM production was analysed as described above. Increased fibroblast activation and production of ECM was observed as compared to non-stimulated cultures in fibroblasts derived from each of the tissues analysed.

### 1.3.1 Liver Fibrosis

To test whether IL-11 signalling is important in liver fibrosis, human primary liver fibroblasts (Cell Biologics, Cat #: H-6019) were cultured at low passage in wells of 96-wall plates and either not stimulated, stimulated with TGFβ1 (5 ng/ml, 24h), IL-11 (5 ng/ml, 24h) or incubated with both TGFβ1 (5 ng/ml) and a neutralising IL-11 antibody (2 μg/ml), or TGFβ1 (5 ng/ml) and an isotype control antibody. Fibroblast activation (αSMA positive cells), cell proliferation (EdU positive cells) and ECM production (periostin and collagen) was analysed using the Operetta platform.

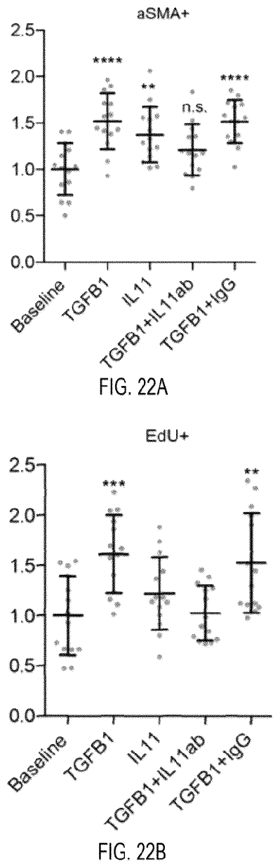

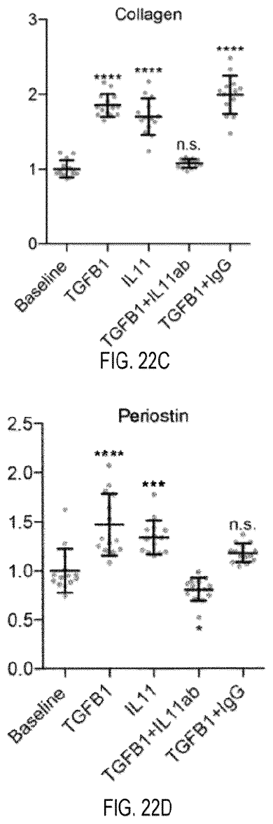

The results of the experiments with primary human ever fibroblasts are shown in FIGS. 22A to 22D. IL-11 was found to activate liver fibroblasts, and IL-11 signalling was found to be necessary for the profibrotic action of TGFβ1 in liver fibroblasts. Both activation and proliferation of fibroblasts was inhibited by neutralising anti-IL-11 antibody.

### 1.3.2 Skin Fibrosis

To test whether IL-11 signalling is important in skin fibrosis, primary mouse skin fibroblasts ware cultured at low passage in wells of 96-well plates and either not stimulated, stimulated with TGFβ1 (5 ng/ml, 24h) or incubated for 24 h with both TGFβ1 (5 ng/ml) and a neutralising IL-11 antibody (2 μg/ml). Fibroblast activation (αSMA positive cells) was then analysed using the Operetta platform.

The results are shown in FIG. 23. TGFβ1-mediated activation of skin fibroblasts was inhibited by neutralising anti-IL-11 antibody.

### 1.3.3 Fibrosis in Multiple Organs

Next, mouse recombinant IL-11 was injected (100 μg/kg, 3 days/week 28 days) into mice to test whether IL-11 can drive global tissue fibrosis in vivo.

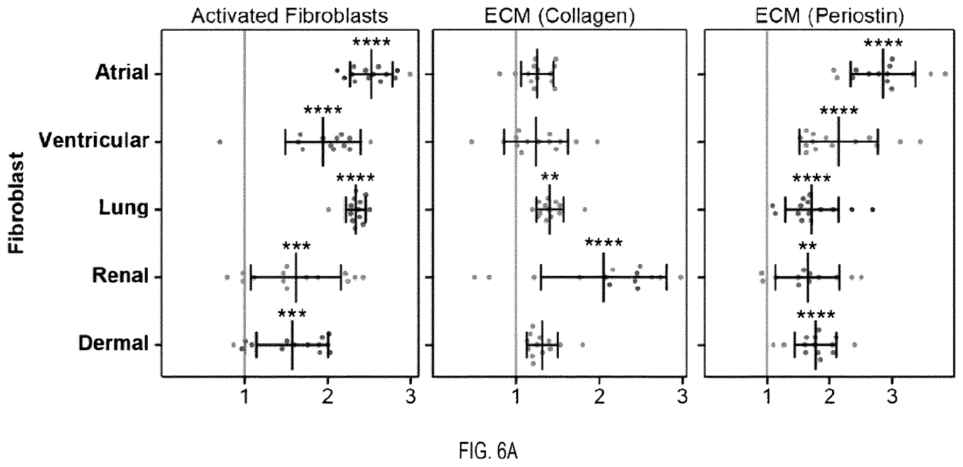

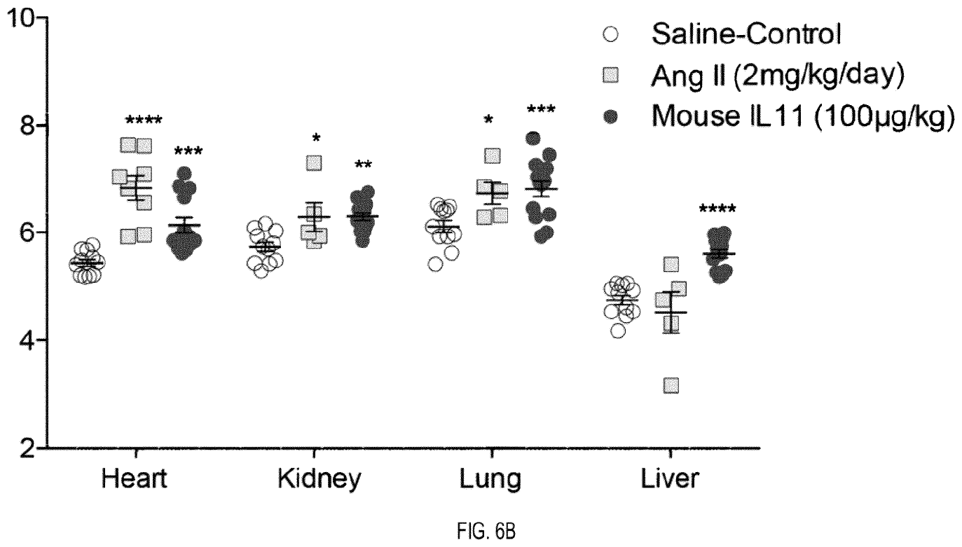

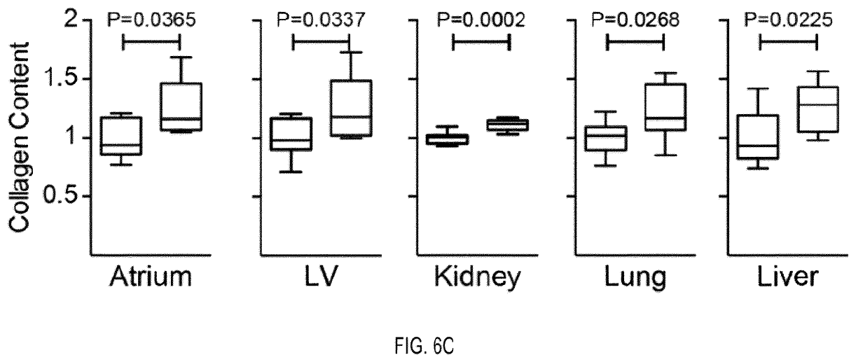

The results are shown in FIG. 6. Compared to injection of AngII (a cytokine that causes an elevation in blood pressure and hypertrophy of the heart), IL-11 also increased the heart weight but also kidney, lung and liver weight indexed to body weight (FIG. 6B). Assessing collagen content in these issues by hydroxyproline assay revealed an upregulation of collagen production in these tissues, indicating fibrosis as the likely cause for the increase in organ weight (FIG. 6C). Expression of fibrosis marker genes ACTA2 (=αSMA), Col1a1, Col3a1, Fn1, Mmp2 and Timp1 was also detected by qPCR analysis of RNA isolated from heart, kidney, lung and liver tissues of these animals.

### Example 2: Therapeutic Potential of IL-11/IL-11R Antagonism

### 2.1 Inhibition of the Fibrotic Response Using Neutralising Antagonists of IL-11/IL-11R

Next it was investigated whether the autocrine loop of IL-11 secretion was required for the profibrotic effect of TGFβ1 an fibroblasts.

IL-11 was inhibited using a commercially available neutralizing antibody (Monoclonal Mouse IgG2A: Clone #22626; Catalog No. MAB218; R&D Systems. MN, USA). Fibroblasts were treated with TGFβ1 in the presence or absence of the antibody, and fibroblast activation, the proportion of proliferating cells and ECM production and markers of the fibrotic response were measured.

Briefly, atrial fibroblasts derived from 3 individuals were incubated fox 24 h with TGFβ1 (5 ng/ml) or TGFβ1 in the presence of neutralising anti-IL-11 antibody or isotype control antibody. Following incubation, cells were stained for αSMA to determine the fraction of myofibroblasts, the proportion of proliferating cells was determined by analysing the cells for EdU incorporation, and periostin was measured to determine ECM production. Fluorescence was measured with the Operetta platform for 14 fields across 2 wells for each individual. Secretion of the fibrosis markers IL-6, TIMP1 and MMP2 was also analysed by ELISA. Fluorescence was normalized to the control group without stimulation.

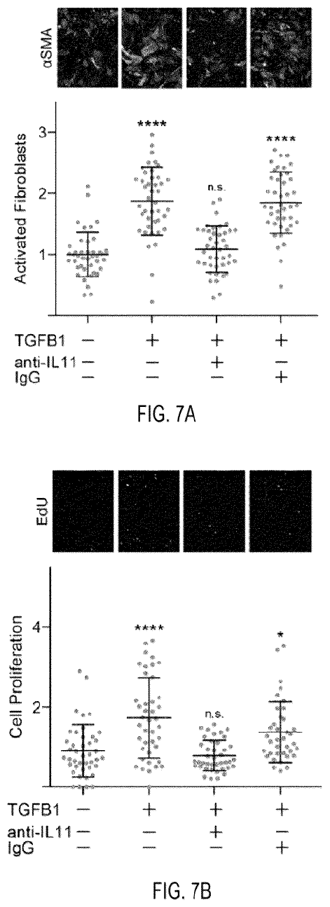

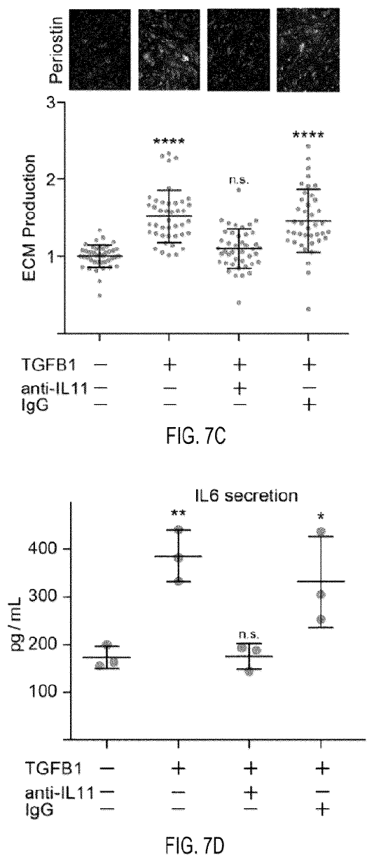

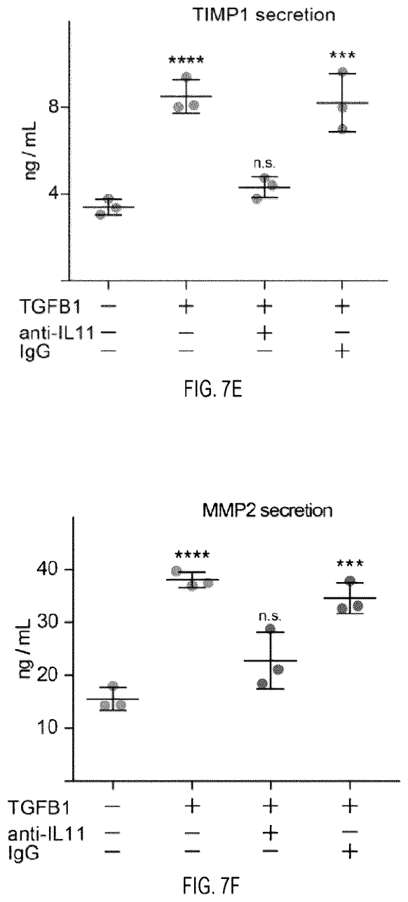

The results are shown in FIGS. 7A to 7F. IL-11 inhibition was found to ameliorate TGFβ1-induced fibrosis, and it was shown that IL-11 is essential for the pro-fibrotic effect of TGFβ1. Inhibition of IL-11 was found to ‘rescue’ the TGFβ1 phenotype at the protein level.

Collagen production was also analysed. Cardiac fibroblasts derived from 3 individuals were incubated for 24 h with TGFβ1 (5 ng/ml) or TGFβ1 and a neutralizing IL-11 antibody. Following incubation the cells were stained for collagen using the Operetta assay and florescence was quantified as described above. Secreted collagen levels in the cell culture supernatant were assessed by Sirius Red staining.

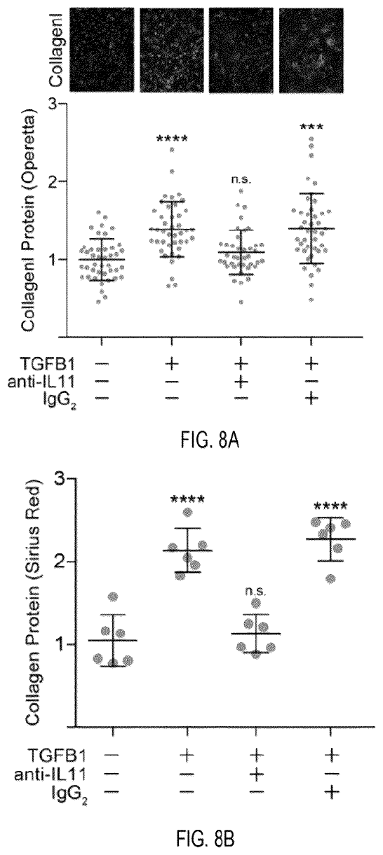

The results are shown in FIGS. 8A and 88, and confirm the anti-fibrotic effect of inhibition of IL-11 using a neutralising antibody.

Next, the ability of several other IL-1/IL-11R antagonists to inhibit fibrosis was analysed in vitro using the atrial fibroblast, TGFβ1-induced myofibroblast transition assay described herein above.

Briefly, human atrial fibroblasts cells were cultured in vitro, stimulated for 24 h with TGFβ1 (5 ng/ml) or left unstimulated, in the presence/absence of (i) neutralising anti-IL-11 antibody, (ii) neutralising anti-IL-11RA antibody, (ii) treatment with siRNA directed against IL-11 or (iv) treatment with siRNA directed against IL-11RA. The proportion of activated fibroblasts (myofibroblasts) was analysed by evaluating αSMA content as described above.

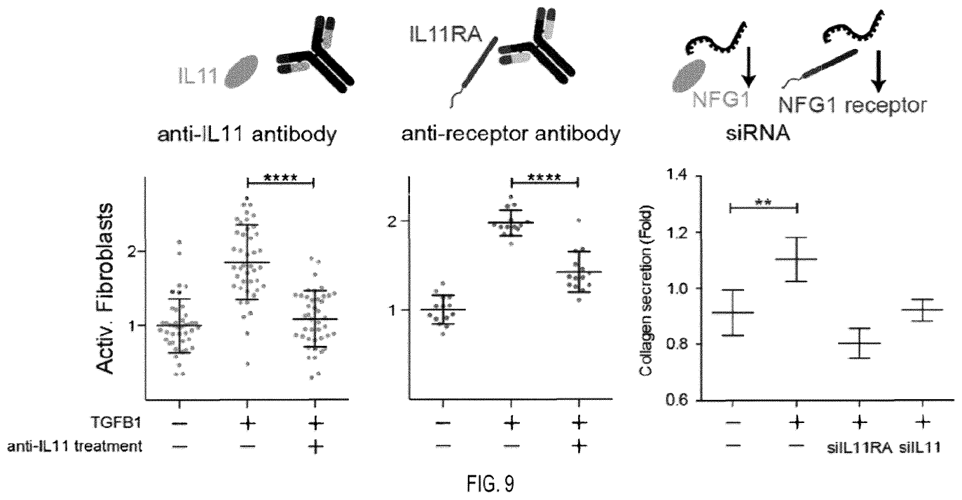

The results are shown in FIG. 9. Each of the antagonists of IL-11/IL-11R signalling was found to be able to abrogate TGFβ1-mediated profibrotic response.

### Example 3: In Vivo Confirmation of a Profibrotic Role for IL-11/IL-11R Signalling

3.1 In Vitro Studies Using Cells Derived from IL-11RA Gene Knock-Out Mice

All mice were bred and housed in the same room and provided food and water ad libitum. Mice lacking functional alleles for IL-11Rα (IL-11RA1 KO mice) were on C57Bl/6 genetic background Mice were of 9-11 weeks of age and the weight of animals did not differ significantly.

To further confirm the anti-fibrotic effect of inhibition of IL-11 signalling, primary fibroblasts were generated from IL-11RA gene knock-out mice and incubated with primary fibroblast eels harvested from IL-11RA+/+ (i.e. wildtype), IL-11RA+/− (i.e. heterozygous knockout) and IL-11RA−/− (i.e. homozygous knockout) animals with TGFβ1, IL-11 or AngII. Activation and proliferation of fibroblasts end ECM production was analysed.

Fibroblasts derived from IL-11RA+/+, IL-11RA+/− and IL-11RA−/− mice were incubated for 24 hours with TGFβ1, IL-11 or AngII (5 ng/mi). Following incubation, cells were stained for αSMA content to estimate the fraction of myofibroblasts, for EdU to identify the fraction of proliferating cels, and for collagen and periostin to estimate ECM production. Fluorescence was measured using the Operetta platform.

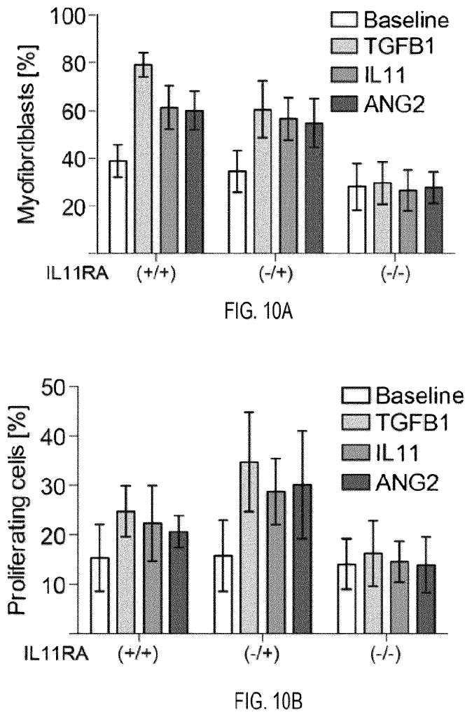

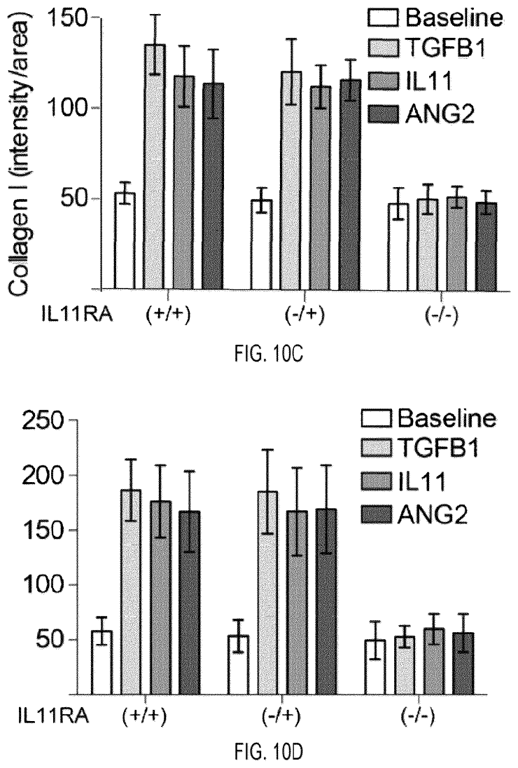

The results are shown in FIGS. 10A to 10D. IL-11RA−/− mice were found not to respond to pro-fibrotic stimuli. These results suggested that IL-11 signalling is also required for AngII-induced fibrosis.

Next, it was investigated whether this was also true tor other pro-fibrotic cytokines. Briefly, fibroblasts were cultured in vitro in the presence/absence of various different pro-fibrotic factors (ANG2, ET-1 or PDGF), and in the presence/absence of neutralising anti-IL-11 antibody or pan anti-TGFβ antibody. After 24 hours, collagen production by the cells was determined by analysis using the Operetta system as described above, and myofibroblast generation was determined by analysis of αSMA expression as described above.

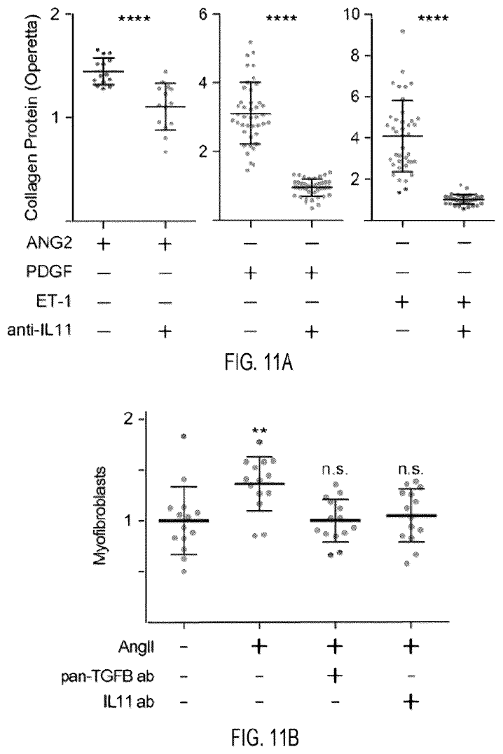

The results are shown in FIGS. 11A and 11B. IL-11 was found to be required for fibrosis downstream of various profibrotic stimuli, and was thus identified as a central mediator of fibrosis induced by a variety of different profibrotic factors.

In a further experiment, the role of IL-11 signalling was investigated in lung fibrosis, using an vitro scratch assay of migration of lung fibroblasts. In response to pro-fibrotic stimuli, fibroblasts are activated and migrate within the fibrotic niche in the body. The migration rate of cells is a measure of cell-cell and cell-matrix interactions and a model for wound healing in vivo (Liang et al., 2007. Nat Protoc. 2(2):329-33).

Fibroblasts derived from lung tissue from both wild type (WT) and also homozygous IL-11RA (−/−) knockout mice were grown al low passage on a plastic surface until they formed a uniform cell monolayer. A scratch was then created in the cell layer, and cell migration close to the scratch was monitored, either in the absence of stimulation, or in the presence of TGFβ1 or IL-11. Images captured at images at the two time points of immediately after creating the scratch and at 24 h were used to determine the area covered by cells, and the rate of migration was compared between WT and KO fibroblasts. Cell migration (area in the scratch covered by cells after 24h) was normalized to the migration rate of WT cells without stimulus.

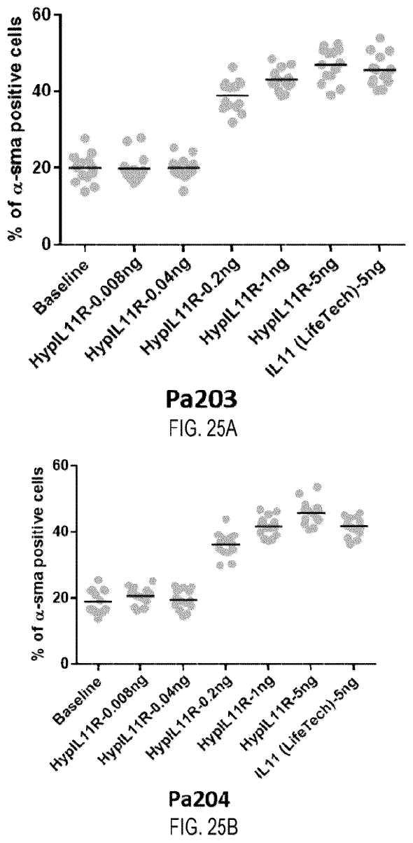

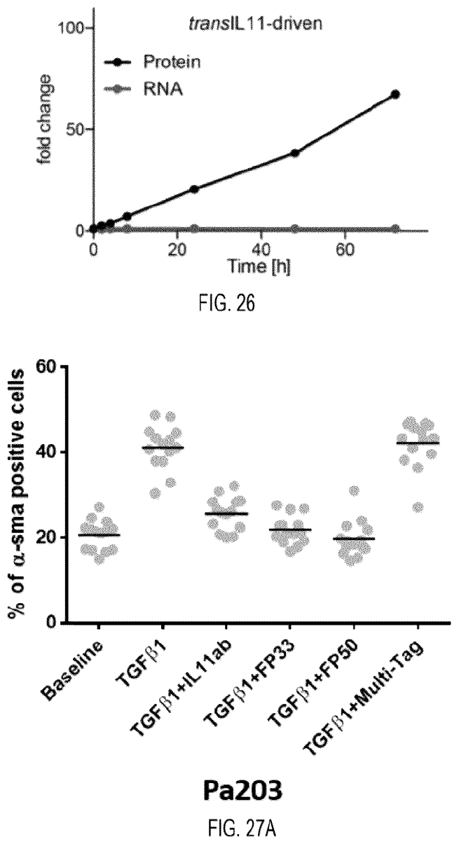

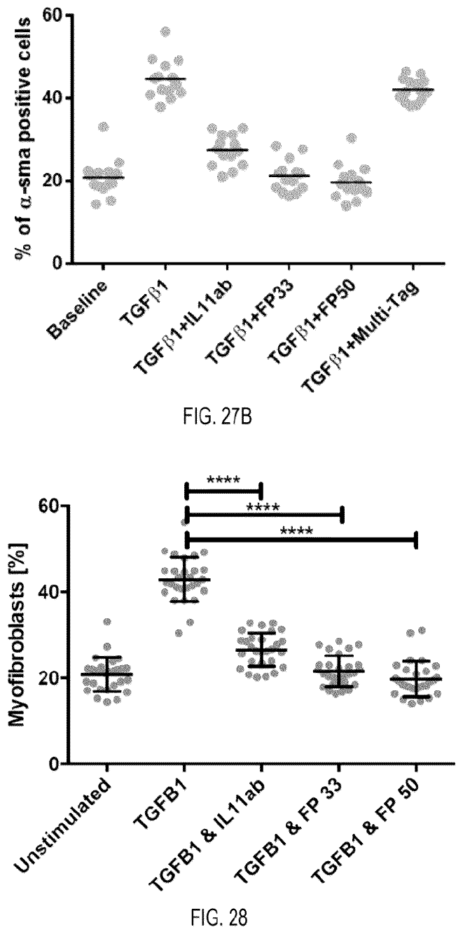

The results are shown in FIG. 24. Lung fibroblasts derived from WT mice were shown to migrate faster in the presence of TGFβ1 and IL-11, indicating a pro-fibrotic effect of both cytokines in lung fibroblasts. Cells lacking IL-11 signalling derived from KG mice migrated more slowly as compared to WT cells. They also did not migrate faster in the presence of TGFβ1. The scratch assay revealed that lung fibroblasts lacking IL-11 signalling have a decrease cell migration rate both in the presence of TGFβ1 or IL-11, and at baseline. Thus, inhibition of IL-11 signalling is anti-fibrotic in the lung.

### 3.2 Heart Fibrosis

The efficacy of IL-11 inhibition to treat fibrotic disorders was investigated in vivo. A mouse model for cardiac fibrosis, in which fibrosis is induced by treatment with AngII, was used to investigate whether IL-11RA−/− mice were protected from cardiac fibrosis.

Briefly, a pump was implanted, and wildtype (WT) IL-11RA(+/+) and knockout (KO) IL-11RA(−/−) mice were treated with AngII (2 mg/kg/day) for 28 days. At the and of the experiment, collagen content was assessed in the atria of the mice using a calorimetric hydroxyproline-based assay kit, and the level of RNA expression of the markers or fibrosis Col1A2, αSMA (ACTA2) and fibronectin (Fn1) were analysed by qPCR.

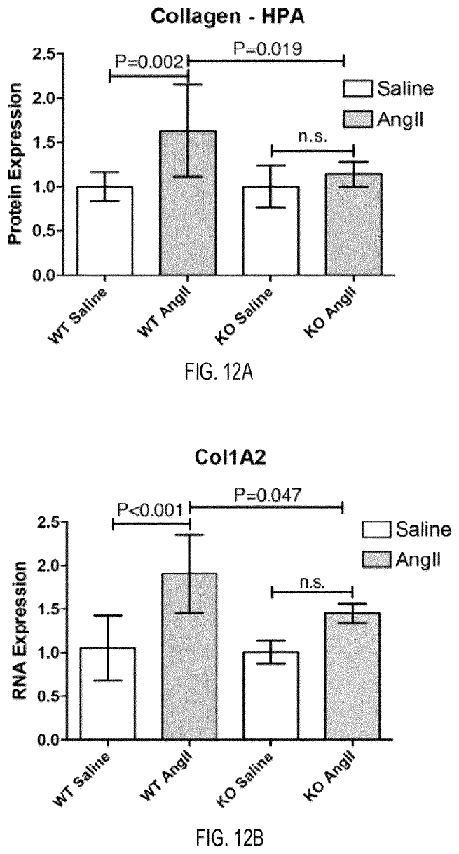

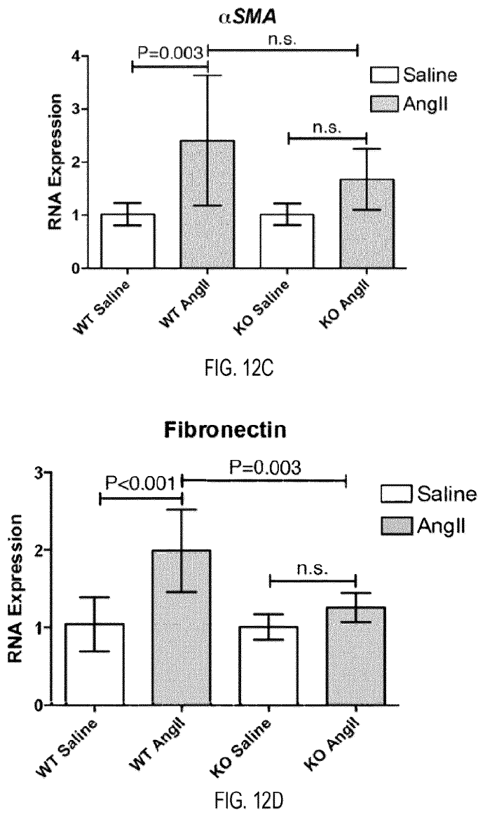

The results are shown in FIGS. 12A to 12D. The IL-11RA−/− mice were found to be protected from the profibrotic effects of AngII.

### 3.3 Kidney Fibrosis

A mouse model for kidney fibrosis was established in wildtype (WT) IL-11RA(+/+) and knockout (KO) IL-11RA(−/−) mice by intraperitoneal injection of folic acid (180 mg/kg) in vehicle (0.3M NaHCO₃); control mice were administered vehicle alone.

Kidneys were removed 28 days post-injection, weighed and either fixed in 10% neutral-buffered formalin for Masson's trichrome and Sirius staining or snap-frozen for collagen assay, RNA, and protein studies.

Total RNA was extracted from the snap-frozen kidney using Trizol reagent (Invitrogen) an Qiagen TissueLyzer method followed by RNeasy column (Qiagen) purification. The cDNA was prepared using iScript™ cDNA synthesis kit; in which each reaction contained 1 μg of total RNA, as per the manufacturer's instructions. Quantitative RT-PCR gene expression analysis was performed on triplicate samples with either TaqMan (Applied Biosystems) or fast SYBR green (Qiagen) technology using StepOnePlus™ (Applied Biosystem) over 40 cycles. Expression data were normalized to GAPDH mRNA expression level and we used the 2-ΔΔCt method to calculate the fold-change. The snap-frozen kidneys were subjected to acid hydrolysis by heating in 6M HCl at a concentration of 50 mg/ml (95° C., 20 hours). The amount of total collagen in the hydrolysate was quantified based on the colormetric detection hydroxyproline using Quickzyme Total Collagen assay kit (Quickzyme Biosciences) as per the manufacture's instructions.

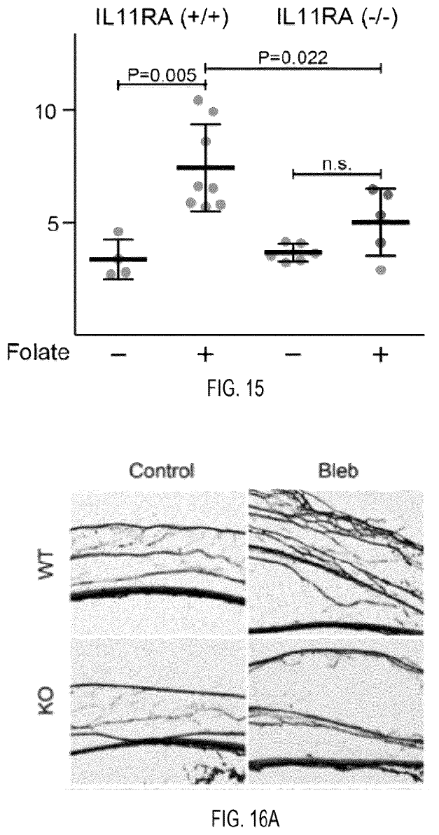

The results of the analysis are shown in FIG. 15. Folate-Induced kidney fibrosis is shown to be dependent on IL-11 mediated signalling. A significant increase in collagen content in kidney tissue was observed in IL-11RA+/+ mice, indicative of kidney fibrosis. No significant increase in collagen content was observed in IL-11RA−− mice. Animals deficient for IL-11 signalling had significantly less collagen deposition in kidneys after toxic injury as compared to wild type animals.

### 3.4 Lung Fibrosis

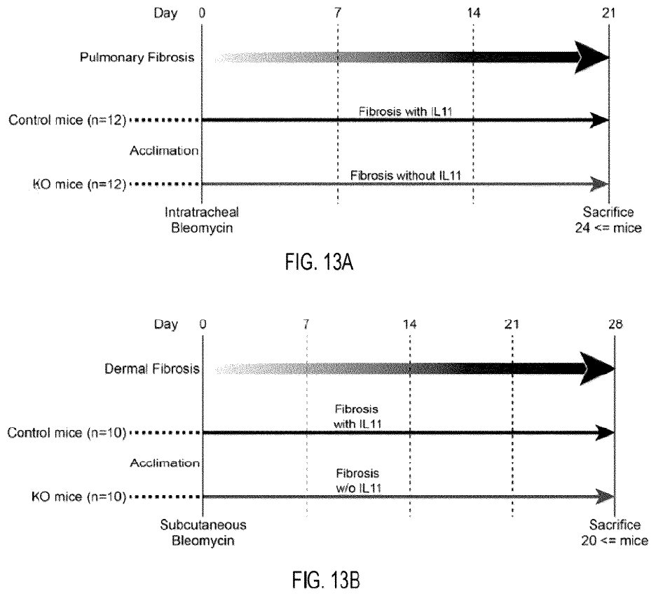

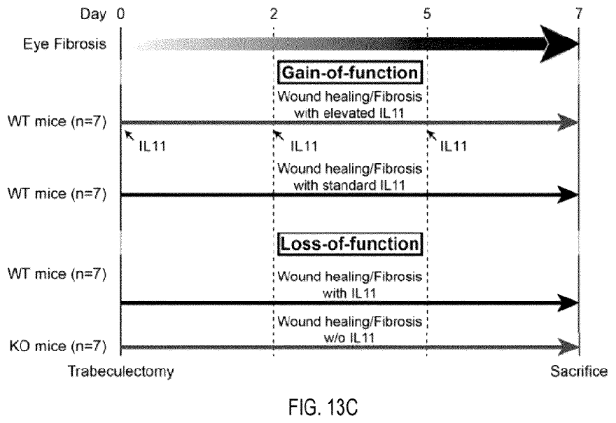

IL-11 is confirmed as a key mediator of fibrosis in the lung, skin and eye in further k vi model using the IL-11RA−/− knockout mice. Schematics of the experiments are shown in FIGS. 13A to 13C.

To analyse pulmonary fibrosis, IL-11RA−/− mice and IL-11RA+/+ mice are treated by intratracheal administration of bleomycin on day 0 to establish a fibrotic response in the lung (pulmonary fibrosis). Fibrosis of the lung develops by 21 days, at which point animals are sacrificed and analysed for differences in fibrosis markers between aminals with and without IL-11 signalling IL-11RA−/− mice have a reduced fibrotic response in lung tissue as compared to IL-11RA+/+ mice, as evidenced by reduced expression of markers of fibrosis.

### 3.5 Skin Fibrosis

To analyse fibrosis of the skin, IL-11RA−/− mice and IL-11RA+/+ mice are treated by subcutaneous administration of bleomycin on day 0 to establish a fibrotic response in the skin. Fibrosis of the skin develops by 28 days, at which point animals are sacrificed and analysed for differences in fibrosis markers between animals with and without IL-11 signalling. IL-11RA−/− mice have a reduced fibrotic response in skin tissue as compared to IL-11RA+/+ mice, as evidenced by reduced expression of markers of fibrosis.

### 3.6 Eye Fibrosis

To analyse fibrosis in the eye, IL-11RA−/− mice and IL-11RA+/+ mice underwent trabeculectomy (filtration surgery) on day 0 to initiate a wound healing response in the eye. This mouse model of glaucoma filtration surgery has been shown to be an efficient model to evaluate the wound healing response in the eye (Khaw et al 2001, Curr Opin Ophthalmol 12, 143-148; Seet et al. 2011, Mol. Med. 17, 557-567) and has successfully shown the beneficial effect of fibrotic modulators in vivo (Mead at al. 2003, Invest. Ophthalmol. Vis. Sci 44, 3394-3401; Wong et al. 2003 Invest. Ophthalmol. Vis. Sci. 44, 1097-1103; Wong et al. 2005. Invest. Opthalmol. Vis. Sci. 46, 2018-2022).

Briefly, the conjunctiva was dissected to expose the underlying sclera, after which an incision was made through the sclera into the anterior chamber of the eye using a 30-gauge needle. The created fistula allowed aqueous humor to exit into and underneath the conjunctiva. The dissected conjunctiva was then secured and closed at the limbus by a 10-0 (0.2 metric) Ethilon black monofilament nylon scleral suture. Fucithalmic ointment was instilled at the end of the procedure. The surgery was performed under anaesthesia by intraperitoneal injection of a 0.1 ml ketamine/xylazine mixture, as well as topical application of one drop per eye of 1% xylocaine. Fucithalmic ointment was instilled post-surgery to prevent infection. Surgery was performed with 70% propyl alcohol sterilized surgical scissors and forceps and sterile needles.

The accumulated fluid underneath the sutured conjunctiva was observed as a conjunctival bleb. Mice were euthanized on day 7 post-surgery for analyses. For qualitative immune-histological analyses, eyes from mice will be harvested by enucleation and then sectioned. Maturation of collagen fibres was evaluated with using the picro-sirius red/polarization light technique (Szendröi et al. 1984, Acta Morphol Hung 32, 47-55); orange-red indicated mature collagen, and yellow/green indicated newly formed immature collagen.

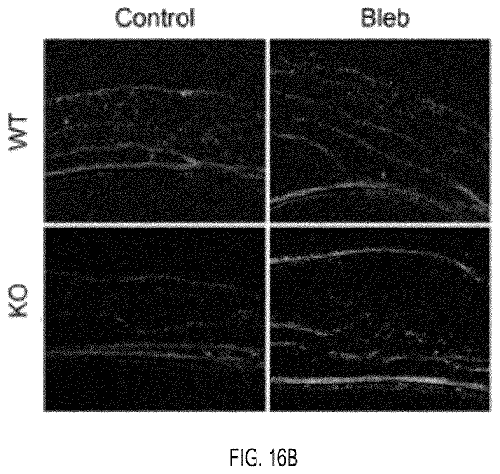

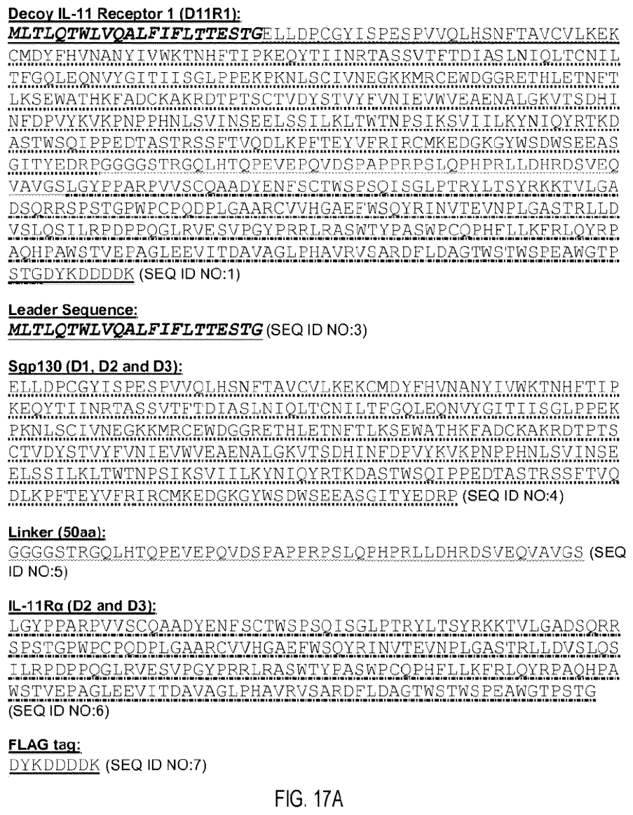

The results of the experiment are shown in FIGS. 16A and 16B. IL-11RA−/− mice were found to have a reduced fibrotic response in eye tissue as compared to IL-11RA+/+ mice.

### 3.7 Other Tissue

The effect of IL-11RA knockout on fibrosa is also analysed in mouse models of fibrosis for other tissues, such as the liver, bowel, and is also analysed in a mode relevant to multiorgan (i.e. systemic) fibrosis. The fibrotic response Is measured and compared between the IL-11RA−/− mice and IL-11RA+/+ mice, IL-11RA−/− mice have a reduced fibrotic response as compared to IL-11RA+/+ mice, as evidenced by reduced expression of markers of fibrosis.

### Example 4: Analysis of the Molecular Mechanisms Underlying IL-11-Mediated Induction of Fibrosis

The canonical mode of action of IL-11 is thought to be regulation of RNA expression via STAT3-mediated transcription (Zhu et al. 2015 PLoS ONE 10, e0126296), and also through activation of ERK.

STAT3 activation is observed following stimulation with IL-11. However, when fibroblasts are incubated with TGFβ1, only activation of the canonical SMAD pathway and ERK pathways is seen, and activation of STAT3 is not observed, even in spite of the fact that IL-11 is secreted in response to TGFβ1. Only ERK activation is common to both TGFβ1 and IL-11 signal transduction.

Cross-talk between TGFβ1 and IL-6 signalling has previously been described, wherein TGFβ1 blocks the activation of STAT3 by IL-8 (Walia et al, 2003 FASEB J 17, 2130-2132). Given the close relationship between IL-6 and IL-11, similar cross-talk may be observed for IL-11 mediated signalling.

The inventors investigated by RNA-seq analysis whether regulation of RNA abundance was the underlying mechanism for the increased expression of fibrosis marker proteins in response to IL-11, which would suggest STAT3 as the underlying signalling pathway for IL-11 mediated profibrotic processes. Fibroblasts were incubated for 24 hours either without stimulus, or in the presence of TGFβ1, IL-1l or TGFβ1 and IL-1.

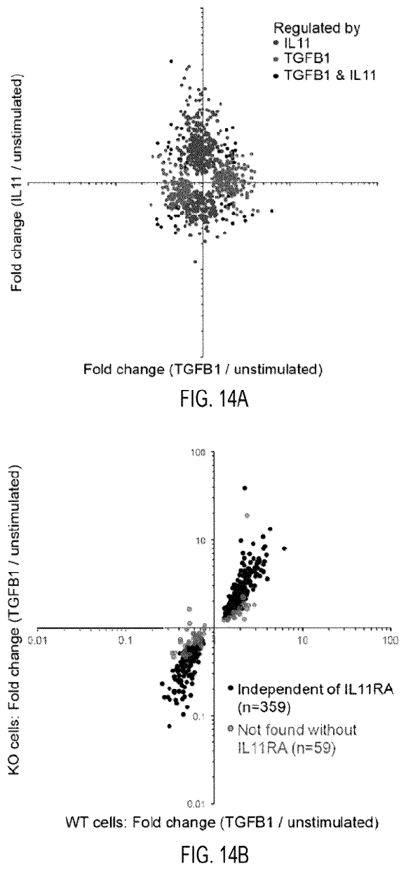

The results are shown in FIG. 14A. TGFβ1 induced the expression of collagen, ACTA2 (αSMA) and other fibrosis marker at the RNA level. However IL-11 did not regulate the expression of these genes, but a different set of genes.

Gene ontology analysis suggests that a pro-fibrotic effect in fibroblasts is driven by IL-11-regulated RNA expression. Both TGFβ1 and IL-11 regulate an almost completely different set of genes or the RNA level.

Whilst TGFβ1 Increases IL-11 secretion, the target genes of IL-11 are not regulated when both TGFβ1 and IL-11 are present. This suggests that TGFβ1 upregulates IL-11 and simultaneously blocks the canonical IL-11-driven regulation of RNA expression via STAT3, similar to what is known about the interaction of TGFβ1 and IL-6 pathways (Walia at al., 2003 FASEB J. 17, 2130-2132).

We also analysed whether RNA expression differences induced by TGFβ1 are dependent on IL-11 signalling, by analysing changes in RNA expression in fibroblasts obtained from IL-11RA−/− mice as compared to IL-11RA+/+ mice. RNA expression regulated by TGFβ1 is still observed when IL-11RA knockout cells were stimulated with TGFβ1, and RNA levels of αSMA, collagen etc. were still upregulated in the absence of IL-11 signalling (in IL-11RA−/− fibroblasts). When the pro-fibrotic effect of IL-11 and the anti-fibrotic effect of IL-11 inhibition was investigated in vitro, reduced expression of markers of fibrosis was only observed at the protein level, not at the transcriptional level as determined by qPCR.

The activation of non-canonical pathways e.g. ERK signal transduction) is known to be crucial for the pro-fibrotic action of TGFβ1 (Guo and Wang, 2008 Cell Res 19, 71-88). It is likely that non-canonical pathways are likely to be important for signalling for all known pro-fibrotic cytokines, and that IL-11 is a post-transcriptional regulator which is essential for fibrosis.

### Example 5: Decoy IL-11 Receptors

### 5.1 Decoy IL-11 Receptor Constructs

Decoy IL-11 Receptor molecules were designed and clones into the pTT5 vector for recombinant expression in 293-6E cells.

Briefly, an insert for the plasmid comprising cDNA encoding the ligand binding domains D1, D2 and D3 of gp130 in-frame with cDNA encoding either a 50 amino add or 33 amino acid linker region, followed by cDNA encoding the ligand binding domains D2 and D3 of human IL-11Rα, followed by cDNA encoding the FLAG tag. The cDNA insert incorporated a leader sequence Kozak sequences at the 5′ end, and included a 5′ EcoRI restriction site and a 3′ HindIII restriction site (downstream cot a slop codon) for insertion into the pTT5 vector.

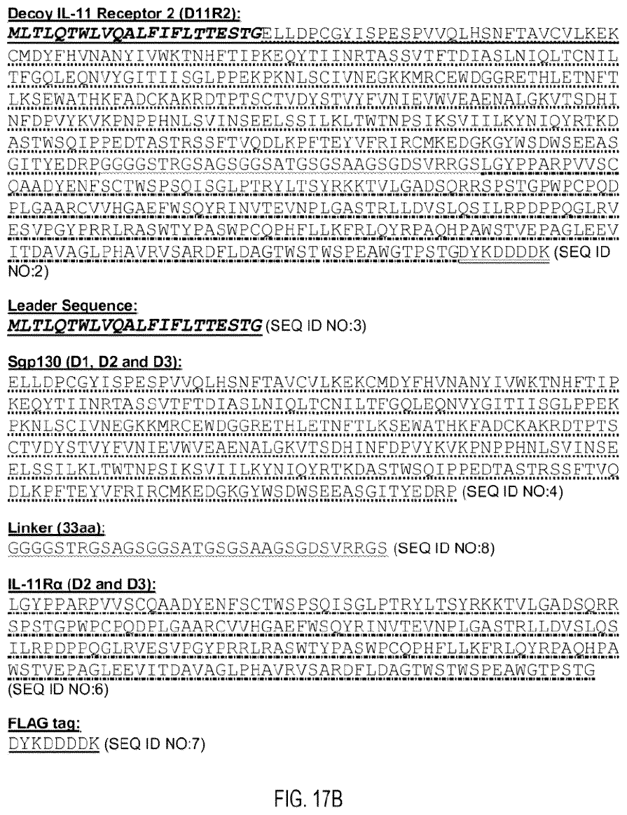

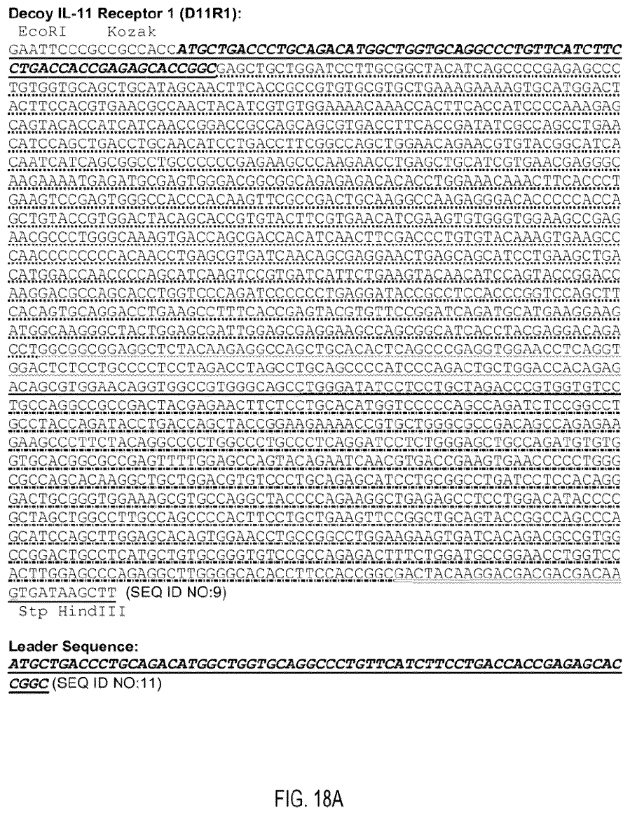

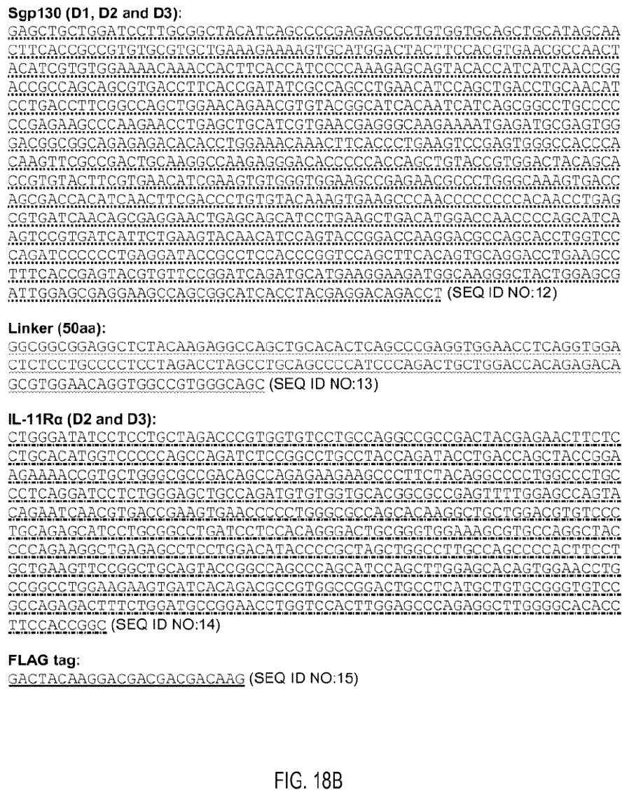

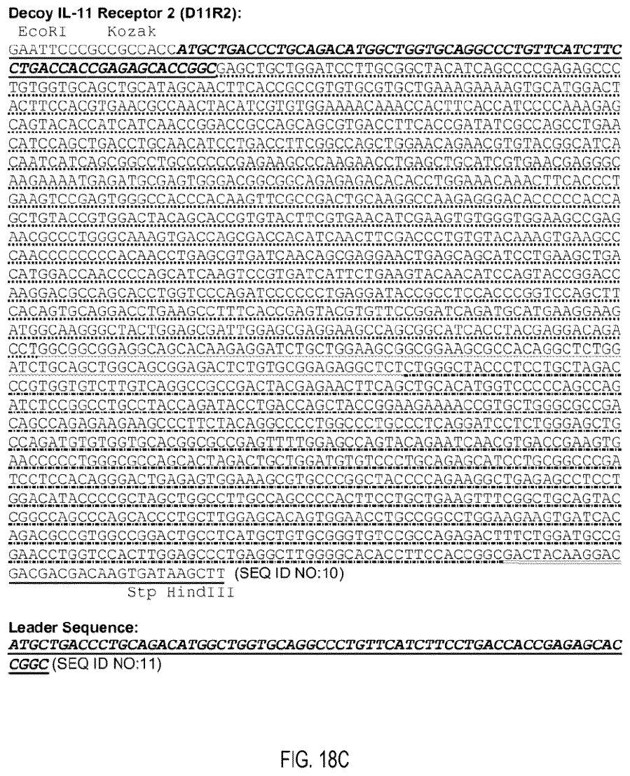

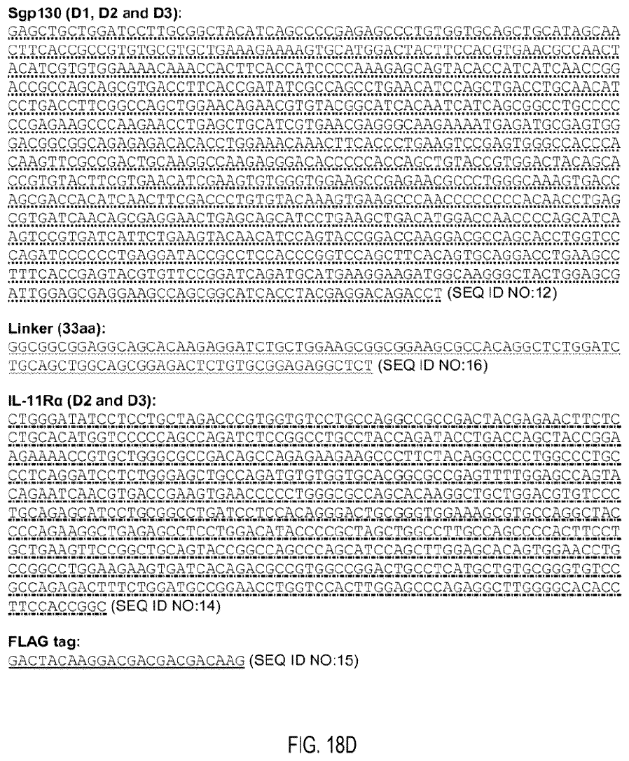

The two constructs encoding a decoy IL-11 receptor molecule having either a 50 amino acid or 33 amino acid sequence are respectively designated Decoy IL-11 Receptor 1 (D11R1) and Decoy IL-11 Receptor 2 (D11R2). The nucleotide sequences for the constructs (and the component sequences thereof) are shown in FIGS. 18A-18D. The amino acid sequences for D11R1 and D11R2 (and the component sequences thereof) are shown in FIGS. 17A and 17B.

### 5.2 Decoy IL-11 Receptor Expression and Purification

The constructs were transfected into 293-6E cells for recombinant expression and purification.

293-6E cells were grown in serum-free FreeStyle™ 293 Expression Medium (Life Technologies, Carlsbad, CA, USA). Cells were maintained in Erlenmeyer Flasks (Corning Inc. Acton, MA) at 37° C. with 5% CO₂ on an orbital shaker (VWR Scientific, Chester, PA).

One day before transfection, the cells were seeded at an appropriate density in Corning Erlenmeyer Flasks. On the day of transfection, DNA and transfection reagent were mixed at an optimal ratio and then added into the flask with cells ready for transfection. The recombinant plasmids encoding D11R1 and D11R2 were transiently transfected into suspension 293-6E cell cultures on two separate days.

Cell culture supernatants were collected on day 6 and used for purification. Briefly, cell cultured broths were centrifuged and filtrated. 0.5 ml of resin was added to cell culture supernatants and incubated for 3-4 hours to capture the target protein.

After washing and elution with appropriate buffers, eluted fractions were analysed by SDS-PAGE and Western blot using Rabbit anti-FLAG polyclonal Ab (GenScript, Cat. No. A00170) to confirm expression of the FLAG-tagged decoy IL-11 receptor molecules.

The purified species were quantified and stored at −80° C.

### Example 6: Functional Characterisation of Decoy IL-11 Receptors

### 6.1 Ability to Inhibit Human IL-11 Mediated Signalling

To investigate ability to neutralise human IL-11-mediated signalling, cardiac atrial human fibroblasts were cultured in wells of 98-well plates in the presence of TGFβ1 (5 ng/ml) for 24 hours, in the presence or absence of various concentrations of D11R1 or D11R2.

TGFβ1 promotes the expression of IL-11 which in turn drives the transition of quiescent fibroblasts to activated, αSMA-positive fibroblasts. It has previously been shown that neutralising IL-11 prevents TGFβ1-induced transition to activated, αSMA-positive fibroblasts.

Expression of αSMA was analysed with the Operetta High-Content Imaging System in an automated high-throughput fashion.

D11R1 or D11R2 were added to fibroblast cultures that were stimulated with TGFβ1 at final concentrations of 5 ng/ml, 50 ng/ml and 500 ng/ml, and at the end of the 24 hour culture period, the percentage of αSMA-positive fibroblasts in the culture was determined.

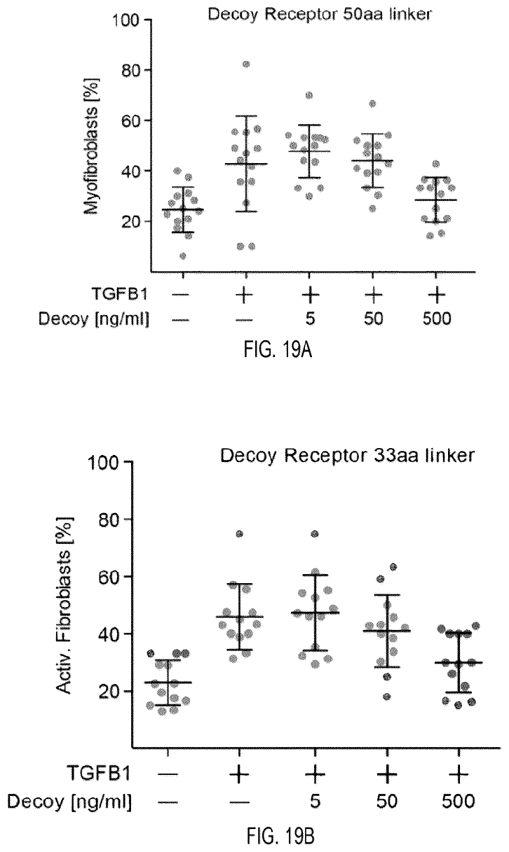

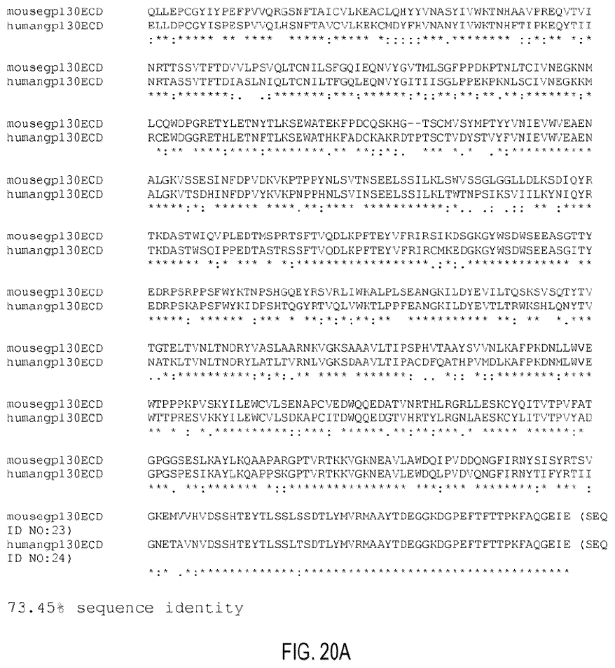

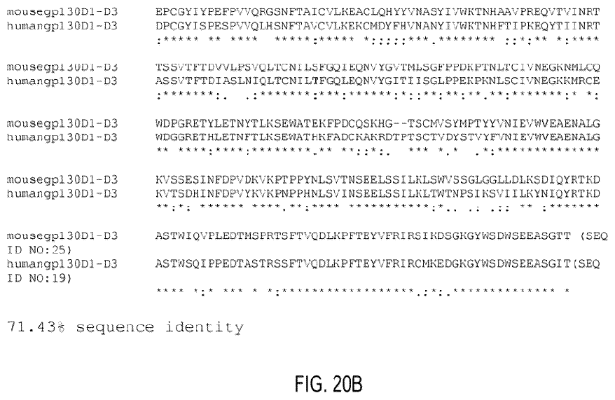

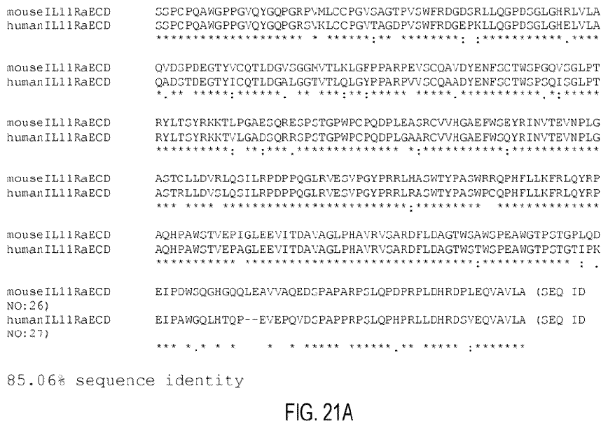

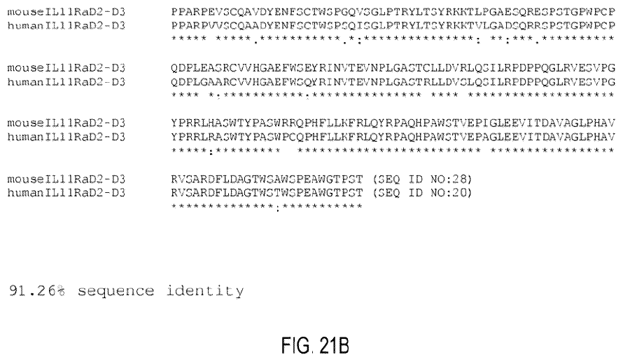

The results of the experiments are shown in FIGS. 19A and 19B. Both D11R1 and D11R2 were demonstrated to be capable of neutralising signalling mediated by human IL-11 in a dose-dependent manner.

The IC₅₀ for the D11R1 and D11R2 molecules was determined to be ˜1 nM.

### 6.2 Ability to Inhibit Mouse IL-11 Mediated Signalling

The ability of D11R1 and D11R2 to inhibit mouse IL-11-mediated signalling is investigated, following the same procedure as described in section 6.1 above, but using mouse dermal fibroblasts instead of human atrial fibroblasts.

D11R1 and D11R2 are demonstrated to be capable of neutralising IL-11/IL-11R signalling in mouse dermal fibroblasts, as determined by observation of a relative decrease in the proportion of αSMA-positive fibroblasts at the end of the 24 h culture period in the presence of D11R1 or D11A2 as compared to culture in the absence of the decoy IL-11 receptors.

6.3 Ability to Inhibit IL-11 Trans Signalling, by IL-11 in Complex with IL-11RA

Trans signalling is recognised as a major aspect of IL-6 signalling, where a complex of IL-6 and soluble IL-6Rα can activate cells that express gp130, but lack the IL-6 receptor (Hunter and Jones, 2015 Nature Immunology 18, 448-457).

It has recently been suggested that trans signalling by a complex of IL-11 and soluble IL-11RA is also important for IL-11 biology (Lokau et al., Call Reports (2016) 14, 1761-1773). Using a recombinant fusion protein of IL-11 and IL-11Rα (as described in Pflanz et al., Febs Lett (1999) 450: 117-122). D11R1 and D11R2 are screened for the ability to Inhibit trans signalling mediated by IL-11:IL-11Rα complex.

Importantly, decoy IL-11 receptors which are capable of inhibiting both classical IL-11 mediated signalling and IL-11 trans signalling by IL-11:IL-11Rα complex are able to inhibit all known modes of IL-11/IL-11R signalling.

The IL-11:IL-11Rα fusion protein (hereafter referred to as hyper IL-11) consists of the extracellular domain of the IL-11 receptor alpha (IL-11Rα) linked to IL-11.

Hyper IL-11 was found to be a more potent activator of human fibroblasts than recombinant IL-11 Protein. Briefly, in two separate experiments human fibroblasts were cultured without stimulation (Baseline). In the presence of different amounts of hyper IL-11 (0.008 ng/mi, 0.04 ng/ml, 0.2 ng/ml, 1 ng/ml and 5 ng/ml), or 5 ng/ml recombinant human IL-11 obtained from a commercial source, aid fibroblast activation was analysed by determining the percentage of αSMA-positive cols as described herein. The results are shown in (FIGS. 25A and 25B). Hyper-IL-11 activated fibroblasts in a dose-dependent fashion, and was a more potent activator than IL-11.

The IL-11:IL-11Rα fusion protein was prepared as follows:
DNA encoding IL-11:IL-11Rα fusion protein (i.e. SEQ ID NO:21) was cloned into pTT5 vector, and transfected into 293-6E cells in culture in serum-free FreeStyle™ 293 Expression Medium (Thermo Fisher Scientific).
Cells were maintained in Erlenmeyer Flasks (Corning Inc.) at 37° C. with 5% CO₂ on an orbital shaker (VWR Scientific).
Cell culture supernatants were collected on day 6 were used for purification.
Cell culture super-tart was loaded onto an affinity purification column.
After washing and elution with appropriate buffer, the eluted fractions were pooled and buffer exchanged to final formulation buffer.
The purified IL-11:IL-11Rα fusion protein was analyzed by SDS-PAGE, Western blot to confirm molecular weight and purity.

DNA encoding IL-11:IL-11Rα fusion protein (SEQ ID NO: 21):
GAATTCCCGCCGCCACCATGGGCTGGTCCTGCATCATCCTGTTTCTGGTGGCCACAGCCACCG
GCGTGCACTCTCCACAGGCTTGGGGACCTCCAGGCGTGCAGTATGGCCAGCCTGGCAGATCC
GTGAAGCTGTGCTGTCCTGGCGTGACAGCTGGCGACCCTGTGTCCTGGTTCAGATGGCGA
GCCCAAGCTGCTGCAGGGCCCAGATTCTGGACTGGGCCACGAACTGGTGCTGGCCCAGGCCG
ATTCTACCGACGAGGGCACCTACATCTGCCAGACCCTGGATGGCGCCCTGGGCGGAACAGTG
ACACTGCAGCTGGGCTACCCTCCCGCCAGACCTGTGGTGTCTTGTCAGGCCGCCGACTACGA
GAACTTCAGCTGCACATGGTCCCCCAGCCAGATCAGCGGCCTGCCCACCAGATACCTGACCAG
CTACCGGAAGAAAACCGTGCTGGGCGCCGACAGCCAGAGAAGAAGCCCTTCTACAGGCCCCT
GGCCCTGCCCTCAGGATCCTCTGGGAGCTGCCAGATGTGTGGTGCACGGCGCCGAGTTCTGG
TCCCAGTACCGGATCAACGTGACCGAAGTGAACCCCCTGGGCGCCTCCACAAGACTGCTGGAT
GTGTCCCTGCAGAGCATCCTGCGGCCCGATCCTCCACAGGGCCTGAGAGTGGAAAGCGTGCC
CGGCTACCCCAGAAGGCTGAGAGCCAGCTGGACATACCCCGCCTCTTGGCCTTGCCAGCCCC
ACTTCCTGCTGAAGTTTCGGCTGCAGTACCGGCCAGCCCAGCACCCTGCTTGGAGCACAGTGG
AACCTGCCGGCCTGGAAGAAGTGATCACAGACGCCGTGGCCGGACTGCCTCATGCTGTGCGG
GTGTCCGCCAGAGACTTTCTGGATGCCGGCACCTGGTCTACCTGGTCCCCAGAAGCCTGGGG
CACACCTTCTACTGGCGGACCTGCTGGACAGTCTGGCGGAGGCGGAGGAAGTGGCGGAGGAT
CAGGGGGAGGATCTGTGCCTGGACCTCCTCCAGGACCCCCTAGAGTGTCCCCAGATCCTAGG
GCCGAGCTGGACTCTACCGTGCTGCTGACCAGATCCCTGCTGGCCGACACAAGGCAGCTGGC
TGCCCAGCTGAGAGACAAGTTCCCCGCCGACGGCGACCACAACCTGGATAGCCTGCCTACCCT
GGCCATGTCTGCTGGCGCACTGGGGGCTCTGCAGCTGCCTGGGGTGCTGACTAGACTGAGAG
CCGACCTGCTGAGCTACCTGCGGCATGTGCAGTGGCTGAGAAGGGCTGGCGGCAGCAGCCTG
AAAACCCTGGAACCTGAGCTGGGCACACTGCAGGCCAGACTGGACAGACTGCTGCGCAGACT
GCAGCTGCTGATGAGCAGACTGGCTCTGCCCCAGCCTCCTCCTGACCCTCCTGCTCCTCCACT
GGCTCCTCCAAGCTCTGCTTGGGGCGGAATTAGAGCCGCCCACGCCATTCTGGGAGGCCTGC
ACCTGACACTGGATTGGGCAGTGCGGGGCCTGCTGCTGCTGAAAACCAGACTGCACCACCAC
CATCACCACTGATAAGCTT
Amino acid sequence of IL-11:IL-11Rα fusion protein (SEQ ID NO: 22):
MGWSCIILFLVATATGVHSPQAWGPPGVQYGQPGRSVKLCCPGVTAGDPVSWFRDGEPKLLQGP
DSGLGHELVLAQADSTDEGTYICQTLDGALGGTVTLQLGYPPARPVVSCQAADYENFSCTWSPSQI
SGLPTRYLTSYRKKTVLGADSQRRSPSTGPWPCPQDPLGAARCVVHGAEFWSQYRINVTEVNPLG
ASTRLLDVSLQSILRPDPPQGLRVESVPGYPRRLRASWTYPASWPCQPHFLLKFRLQYRAQHPA
WSTVEPAGLEEVITDAVAGLPHAVRVSARDFLDAGTWSTWSPEAWGTPSTGGPAGQSGGGGSG
GGSGGGSVPGPPPGPPRVSPDPRAELDSTVLLTRSLLADTRQLAAQLRDKFPADGDHNLDSLPTLA
MSAGALGALQLPGVLTRLRADLLSYLRHVQWLRRAGGSSLKTLEPELGTLQARLDRLLRRLQLLMS
RLALPQPPPDPPAPPLAPPSSAWGGIRAAHAILGGLHLTLDWAVRGLLLLKTRLHHHHHH

Fibroblasts cultured in vitro and stimulated with hyper IL-11 were shown to upregulate IL-11 protein expression, as determined by ELISA (FIG. 26). Interestingly, an increase in IL-11 RNA level was not detected in response to stimulation with hyper IL-11. Unlike TGFB1, which increases IL-11 expression at both the RNA ani the protein level hyper IL-11 seems to upregulate IL-11 expression only post-transcriptionally, at the protein level.

The ability of the D11R1 and D11R2 decoy IL-11 receptors to Inhibit signalling mediated by hyper IL-11 is investigated.

Human atrial fibroblasts are incubated for 24h in the presence of hyper IL-11 (0.2 ng/ml), and in the presence D11R1 or D11R2. Following incubation, cells are stained for αSMA to determine the fraction of myofibroblasts. Stimulation with hyper IL-11 causes a ˜2-fold increase in the percentage of activated fibroblasts as compared to non-stimulated cultures.

D11R1 and D11R2 are demonstrated to be capable of inhibiting IL-11 trans signalling, s determined by observation of a relative decrease in the proportion of αSMA-positive fibroblasts at the er of the 24 h culture period in the presence of D11R1 or D11R2 as compared to culture in the absence of the decoy IL-11 receptors.

### 6.4 Analysis of Decoy IL-11 Receptor Affinity for IL-11

D11R1 and D11R2 are analysed for their affinity of binding to human IL-11 by ELISA assay.

Recombinant human IL-11 was obtained from Genscript and Horseradish peroxidase (HRP)-conjugated anti-FLAG antibody is obtained. Coming 96-well ELISA plates were obtained from Sigma. Pierce 3,3′,5,5′-tetramethylbenzidine (TMB) ELISA substrate kit was obtained from Life Technologies (0.4 g/mL TMB solution, 0.02% hydrogen peroxide in citric acid buffer). Bovine serum albumin and sulphuric acid was obtained from Sigma. Wash buffer comprised 0.05% Tween-20 in phosphate buffered saline (PBS-T). Tecan Infinite 200 PRO NanoQuant is used to measure absorbance.

An indirect EUSA is performed to assess the binding affinity of D11R1 and D11R2 at 50% of effective concentration (EC₅₀) as previously described (Unverdorben et al., (2016) MAbs 8, 120-128). ELISA plates are coated with 1 μg/mL of recombinant human IL-11 overnight at 4° C. and remaining binding sites were blocked with 2% BSA in PBS. D11R1 and D11R1 are diluted in 1% BSA in PBS, titrated to obtain working concentrations of 800, 200, 50, 12.5, 3.125, 0.78, 0.195, and 0.049 ng/mL, and incubated in duplicates for 2 hours at room temperature. Detection of antigen-decoy IL-11 receptor binding is performed with HRP-conjugated anti-FLAG antibody. Following 2 hours of incubation with the detection antibody, 100 μl of TMB substrate its added for 15 mins and chromogenic reaction stopped with 100 μl of 2 M H₂SO₄. Absorbance reading is measured at 450 nm with reference wavelength convection at 570 nm. Data are fitted with GraphPad Prism software with log transformation of decoy IL-11 receptor concentration followed by non-linear regression analysis with the asymmetrical (five-parameter) logistic dose-response curve to determine EC50 values.

The same materials and procedures as described above were performed to determine the affinity of binding to recombinant murine IL-11 obtained from Genscript.

### 6.5 Ability to Inhibit Human IL-11 Mediated Signalling a Variety of Tissues

Ability of the decoy IL-11 receptors D11R1 end D11R2 to neutralise IL-11-mediated signalling and trans signalling in fibroblasts obtained from a variety of different tissues is investigated, essentially as described in sections 6.1 and 6.3 except that instead of cardiac atrial human fibroblasts, human fibroblasts derived from liver, tung, kidney, eye, skirt, pancreas, spleen, bowel, brain, and bone marrow are used for the experiments.

D11R1 and D11R2 are demonstrated to be capable of neutralising signalling in fibroblasts derived from the various different tissues, as determined by observation of a relative decrease in the proportion of αSMA-positive fibroblasts t the end of the 24 h culture period in the presence of the decoy IL-11 receptors as compared to culture in the absence of the decoy IL-11 receptors.

### 6.6 Comparison of Ability of Decoy IL-11 Receptors to Inhibit IL-11 Mediated Signalling as Compared to a Commerically Available Anti-IL-11 Antibody

The ability of D11R1 and D11R2 to inhibit IL-11 mediated signalling was compared to inhibition by the commercially available mouse monoclonal anti-IL-11 antibody clone #22626 (Catalog No. MAB218; R&D Systems, MN, USA).

Briefly, human atrial fibroblasts were cultured in the absence of stimulation or with 5 ng/ml TGFβ1 for 24 hours, in the presence or absence of the commercial anti-IL-11 antibody. D11R1 (FP50), D11R2 (FP33) or in the presence of a mufti-tag fusion protein control. Fibroblast activation was analysed by determining the percentage of αSMA positive cells.

The results of two different experiments are shown in FIGS. 27A and 27B. Both D11R1 and D11R2 were found to be able to inhibit IL-11 mediated signalling to a greeter extent than the commercial anti-IL-11 antibody, and to completely abrogate fibroblast activation in response to stimulation with TGFβ1.

In a further experiment it was investigated as to whether 500 ng/ml of Decoy receptor would be sufficient to inhibit IL-11 signalling. Briefly, human atrial fibroblasts were cultured in the absence of stimulation or with 6 ng/ml TGFβ1 for 24 hours, in the presence or absence of 2 μg/ml of the commercial anti-IL-11 antibody, 500 ng/ml of D11R1 (FP50), or 500 ng/ml of D11R2 (FP33). Fibroblast activation was analysed by determining the percentage of αSMA positive cells.

The results are shown in FIG. 2B. Both D11R1 and D11R2 were found to completely abrogate fibroblast activation in response to stimulation with TGFβ1 even at a concentration as low as 500 ng/ml, and both decoy receptors were found to be more effective at inhibiting fibroblast activation than the commercial anti-IL-11 antibody, even when the antibody was used at tour times the concentration of the decoy receptors.

### Example 7: Further Biochemical Analysis of the Decoy IL-11 Receptors

D11R1 and D11R2 described above are subjected to further biochemical analysis.

The decoy IL-11 receptors are analysed by BIAcore. Biolayer interferometry (BLI) and MicroScale Thermophoresis (MST) analysis to determine the affinity of binding to human IL-11 and mouse IL-11.

BIAcore determination of affinity by surface plasmon resonance (SPR) analysis is performed as described in Rich et al., Anal Biochem. 2008 Feb. 1; 373(1):112-20.

Biolayer interferometry analysis of affinity is performed as described in Concepcion et al., Comb Chem High Throughput Screen. 2009 September; 12(8):791-800.

MicroScale Thermophoresis analysis of affinity is performed as described in Jerabek-Willemsen et al., Assay Drug Dev Technol. 2011 August; 9(4): 342-353.

Aggregation of the decoy IL-11 receptors is analysed by size exclusion chromatography (SEC), as described in Jacob et al., J Pharm Sci. 2013 December; 102(12): 4315-4329.

Hydrophobicity of the decoy IL-11 receptors to analysed by Hydrophobic interaction chromatography (HIC) as described in Haverick of a MAbs. 2014 July-August; 6(4):852-8.

The melting temperature of the decoy IL-11 receptors is analysed by Differential scanning fluorimetry (DSF) as described in Menzen and Friess, J Pharm Sci 2013 February; 102(2):415-28.

### Example 8: Inhibition of Fibrosis In Vivo Using Decoy IL-11 Receptors

The therapeutic utility of the decoy IL-11 receptor is demonstrated in in vivo mouse models of fibrosis for various different tissues. The mice used in the experiments are widtype (i.e. IL-11RA+/+) mice.

### 8.1 Heart Fibrosis

A pump is implanted, and mice are treated with AngII (2 mg/kg/day) for 28 days.

Decoy IL-11 receptors D11R1 or D11R2 ere administered to different groups of mice by intravenous injection. At the end of the experiment, collagen content is assessed in the atria of the mice using a calorimetric hydroxyproline-based assay kit, and the level of RNA expression of the markers or fibrosis Col1A2, αSMA (ACTA2) and fibronectin (Fn1) were analysed by qPCR.

Mice treated with decoy IL-11 receptors have a reduced fibrotic response in heart tissue as compared to untreated/vehicle treated controls, as evidenced by reduced expression of markers of fibrosis.

### 8.2 Kidney Fibrosis

A mouse model for kidney fibrosis is established, in which fibrosis is induced by intraperitoneal injection of folic acid (180 mg/kg) in vehicle (0.3M NaHCO₃); control mice were administered vehicle alone.

Decoy IL-11 receptors D11R1 or D11R2 are administered to different groups of mice by intravenous injection. Kidneys are removed at day 28, weighed and either fixed in 10% neutral-buffered formalin for Masson's trichrome and Sirius staining or snap-frozen or collagen assay, RNA, and protein studies.

Total RNA is extracted from the snap-frozen kidney using Trizol reagent (Invitrogen) and Qiagen TissueLyser method followed by RNeasy column (Qiagen) purification. The cDNA is prepared using iScript™ cDNA synthesis kit, in which each reaction contained 1 μg of total RNA, as per the manufacturer's instructions. Quantitative RT-PCR gene expression analysis is performed on triplicate samples with either TaqMan (Applied Biosystems) or fast SYBR green (Qiagen) technology using StepOnePlus™ (Applied Biosystem) over 40 cycles. Expression data are normalized to GAPDH mRNA expression level and the 2-ΔΔCt method is used to calculate 0h fold-change. The snap-frozen kidneys are subjected to acid hydrolysis by heating in 6M HCl at a concentration of 50 mg/mi (9° C., 20 hours). The amount of total collagen in the hydrolysate is quantified based on the colorimetric detection of hydroxyproline using Quickzyme Total Collagen assay kit (Quickzyme Biosciences) as per the manufacturer's instructions.

Mice treated with decoy IL-11 receptors have a reduced fibrotic response in kidney tissue as compared to untreated/vehicle treated controls, as evidenced by reduced expression of markers of fibrosis.

### 8.3 Lung Fibrosis

Mice are treated by intratracheal administration of bleomycin on day 0 to establish a fibrotic response in the lung (pulmonary fibrosis).

Decoy IL-11 receptors D11R1 or D11R2 are adminstered to different groups of mice by intravenous injection. Mice are sacrificed at day 21, and analysed for differences in fibrosis markers.

Mice treated with decoy IL-11 receptors have a reduced fibrotic response in lung tissue as compared to untreated/vehicle treated controls, as evidenced by reduced expression of markers of fibrosis.

### 8.4 Skin Fibrosis

Mice are treated by subcutaneous administration of bleomycin on day 0 to establish a fibrotic response in the skin.

Decoy IL-11 receptors D11R1 or D11R2 are administered to different groups at mice by intravenous injection. Mice are sacrificed at day 21, and analysed for differences in fibrosis markers.

Mice treated with decoy IL-11 receptors have a reduced fibrotic response in skin tissue as compared to untreated/vehicle treated controls, as evidenced by reduced expression of markers of fibrosis.

### 8.5 Eye Fibrosis

Mice undergo trabeculectomy procedure as described in Example 3.6 above to initiate a wound healing response in the eye.

Decoy IL-11 receptors D11R1 or D11R2 are administered to different groups of mice by intravenous injection, and fibrosis is monitored in the eye tissue.

Mice treated with decoy IL-11 receptors have a reduced fibrotic response in eye tissue as compared to untreated/vehicle treated controls, as evidenced by reduced expression of markers of fibrosis.

### 8.6 Other Tissues

The effect of treatment with decoy IL-11 receptors D11R1 or D11R2 on fibrosis is also analysed in mouse models of fibrosis for other tissues, such as the liver, kidney, bowel, and is also analysed in a model relevant to multiorgan (i.e. systemic) fibrosis.

The fibrotic response is measured and compared between mice treated with decoy IL-11 receptors and untreated mice, or vehicle treated controls. Mice treated with decoy IL-11 receptors have a reduced fibrotic response as compared to untreated/vehicle treated controls, as evidenced by reduced expression of markers of fibrosis.

### Example 9: Treatment of Cancer In Vivo Using Decoy IL-11 Receptors

The effect of treatment with decoy IL-11 receptors D11R1 or D11R2 on cancer is analysed in mouse models of cancer.

Models of breast, lung, and gastrointestinal cancers are established in mice, the mice are treated by administration of decoy IL-11 receptors, and the development/progression of cancer is monitored.

An anti-cancer effect is observed for the decoy IL-11 receptors, as evidenced by reduced symptoms of cancer and/or increased survival as compared to untreated/vehicle treated controls.

### Example 10: Treatment of AMD Using Decoy IL-11 Receptors

The effect of treatment with decoy IL-11 receptors D11R1 or D11R2 is investigated in wet age-related macular degeneration (AMD).

Decoy IL-11 receptors D11R1 or D11R2 are administered to subjects having wet AMD. In some treatment conditions, subjects are administered with VEGF antagonist therapy (e.g. ranibizumab, bevacizumab pegaptanib, brolucizumab or aflibercept), PDGF antagonist therapy (e.g. pegpleranib), or are treated by laser coagulation therapy in addition to treatment with decoy IL-11 receptor.

A reduction in wet AMD pathology and/or improvement in the symptoms of wet AMD observed in subjects treated with decoy IL-1l receptors as compared to subjects not treated with decoy IL-11 receptor.

1-35. (canceled)

36. A method of treating a disease or condition in which IL-11 mediated signalling is pathologically-implicated, comprising administering an IL-11 binding receptor to a subject suffering from the disease or condition, wherein the IL-11 binding receptor comprises (i) an amino acid sequence having at least 70% sequence identity to the cytokine binding module (CBM) of gp130, and (ii) an amino acid sequence having at least 70% sequence identity to the cytokine binding module (CBM) of IL-11Rα, and wherein the IL-11 binding receptor is capable of binding to IL-11 and inhibiting IL-11 mediated signalling.

37. The method according to claim 36, wherein the IL-11 binding receptor is capable of inhibiting interaction between: (i) IL-11 and gp130, or (ii) IL-11 and IL-11Rα.

38. The method according to claim 36, wherein the IL-11 binding receptor comprises an amino acid sequence having at least 70% sequence identity to the sequence of SEQ ID NO:19.

39. The method according to claim 36, wherein the IL-11 binding receptor comprises an amino acid sequence having at least 70% sequence identity to the sequence of SEQ ID NO:20.

40. The method according to claim 36, wherein the IL-11 binding receptor comprises:
(i) an amino acid sequence having at least 70% sequence identity to the sequence of SEQ ID NO: 19; and
(ii) an amino acid sequence having at least 70% sequence identity to the sequence of SEQ ID NO: 20.

41. The method according to claim 36, wherein the IL-11 binding receptor comprises an amino acid sequence having at least 60% sequence identity to the sequence of SEQ ID NO:1 or SEQ ID NO:2.

42. The method according to claim 36, wherein the IL-11 binding receptor comprises a sequence encoding a protein tag, optionally wherein the protein tag is a FLAG, His, Myc, GST, MBP, HA, E, or Biotin tag.

43. The method according to claim 36, wherein the IL-11 binding receptor comprises the amino acid sequence of SEQ ID NO: 1 or SEQ ID NO:2.

44. The method according to claim 36, wherein the subject suffering from a disease or condition in which IL-11 mediated signalling is pathologically-implicated is a human subject.

45. The method according to claim 36, wherein the disease or condition in which IL-11 mediated signalling is pathologically-implicated is fibrosis, or a disease characterized by fibrosis.

46. The method according to claim 45, wherein the fibrosis is fibrosis of the lung, liver, heart, kidney, blood vessels, small intestine, large intestine, pancreas, eye, brain, skin, bone marrow, and/or muscle tissue.

47. The method according to claim 45, wherein the disease characterized by fibrosis is selected from the group consisting of: pulmonary fibrosis, cystic fibrosis, idiopathic pulmonary fibrosis, progressive massive fibrosis, scleroderma, obliterative bronchiolitis, Hermansky-Pudlak syndrome, asbestosis, silicosis, chronic pulmonary hypertension, AIDS-associated pulmonary hypertension, sarcoidosis, tumor stroma in lung disease, asthma, cardiac fibrosis, myocardial fibrosis, atrial fibrosis, ventricular fibrosis, atrial fibrillation, ventricular fibrillation, myocardial infarction, hypertrophic cardiomyopathy, dilated cardiomyopathy, Brugada syndrome, myocarditis, endomyocardial fibrosis, fibrotic vascular disease, hypertensive heart disease, arrhythmogenic right ventricular cardiomyopathy, hepatic fibrosis, chronic liver disease, liver cirrhosis, non-alcoholic steatohepatitis, primary biliary cirrhosis, primary sclerosing cholangitis, schistosomal liver disease, intestinal fibrosis, Crohn's disease, microscopic colitis, pancreatic fibrosis, renal fibrosis, chronic kidney disease, tubulointerstitial fibrosis, glomerular fibrosis, nephritic syndrome, Alport's syndrome, HIV associated nephropathy, polycystic kidney disease, Fabry's disease, diabetic nephropathy, chronic glomerulonephritis, nephritis associated with systemic lupus, eye fibrosis, Grave's opthalmopathy, epiretinal fibrosis, retinal fibrosis, subretinal fibrosis, macular degeneration, wet age-related macular degeneration, diabetic retinopathy, glaucoma, corneal fibrosis, post-surgical fibrosis of the posterior capsule following cataract surgery, post-surgical fibrosis of the bleb following trabeculectomy, conjunctival fibrosis, subconjunctival fibrosis, gliosis, Alzheimer's disease, skin fibrosis, scleroderma, nephrogenic systemic fibrosis, cutis keloid, Dupuytren's contracture, myelofibrosis, muscular dystrophy, Duchenne muscular dystrophy, Becker's muscular dystrophy (BMD), arthritis, adhesive capsulitis, mediastinal fibrosis, retroperitoneal fibrosis, Peyronie's disease, systemic sclerosis, progressive systemic sclerosis, chronic graft versus host disease, fibrotic pre-neoplastic disease, fibrotic neoplastic disease, and fibrosis induced by chemical or environmental insult.

48. The method according to claim 45, wherein the disease characterized by fibrosis is selected from the group consisting of: pulmonary fibrosis, cystic fibrosis, idiopathic pulmonary fibrosis, progressive massive fibrosis, scleroderma, obliterative bronchiolitis, Hermansky-Pudlak syndrome, asbestosis, silicosis, chronic pulmonary hypertension, AIDS-associated pulmonary hypertension, sarcoidosis, tumor stroma in lung disease and asthma.

49. The method according to claim 45, wherein the disease characterized by fibrosis is selected from the group consisting of: hepatic fibrosis, chronic liver disease, liver cirrhosis, non-alcoholic steatohepatitis, primary biliary cirrhosis, primary sclerosing cholangitis and schistosomal liver disease.

50. The method according to claim 36, wherein the disease or condition in which IL-11 mediated signalling is pathologically-implicated is a cancer.

51. The method according to claim 50, wherein the cancer is selected from the group consisting of: an epithelial cell cancer, breast cancer, gastrointestinal cancer, esophageal cancer, stomach cancer, pancreatic cancer, liver cancer, hepatocellular cancer, gallbladder cancer, colorectal cancer, colonic cancer, anal cancer, gastrointestinal carcinoid tumor, lung cancer, lung adenocarcinoma, non-small cell lung cancer and small cell lung cancer.

52. The method according to claim 36, wherein the disease in which IL-11 mediated signalling is pathologically-implicated is inflammation or a disease characterized by inflammation.

53. The method according to claim 52, wherein the disease characterized by inflammation is selected from the group consisting of: allergic inflammation, allergic asthma, bronchial inflammation, atopic dermatitis, allergic rhinitis, an ocular allergic disease, an autoimmune disease, multiple sclerosis, systemic lupus erythematosus, rheumatoid arthritis, chronic active hepatitis, type 1 diabetes mellitus, celiac disease, Grave's disease, uveitis, pemphigus, psoriasis, Crohn's disease, ulcerative colitis, inflammatory bowel disease, anemia and autoimmune thyroiditis.

54. The method according to claim 36, wherein the subject suffering from a disease or condition in which IL-11 mediated signalling is pathologically-implicated is a subject in which expression of IL-11 or IL-11Rα is upregulated.


SEQUENCE LISTING (28 sequences)

SEQ ID NO: 1 - protein, 594 aa, synthetic construct
  REGION 1..594: note = Decoy IL-11 Receptor 1 (D11R1):
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPGGGGSTRGQLHTQPEVEPQVDSPAPPRPSLQPHPRLLDHRDSVEQVAVGSLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGDYKDDDDK

SEQ ID NO: 2 - protein, 577 aa, synthetic construct
  REGION 1..577: note = Decoy IL-11 Receptor 2 (D11R2):
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPGGGGSTRGSAGSGGSATGSGSAAGSGDSVRRGSLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGDYKDDDDK

SEQ ID NO: 3 - protein, 22 aa, synthetic construct
  REGION 1..22: note = leader sequence
MLTLQTWLVQALFIFLTTESTG

SEQ ID NO: 4 - protein, 304 aa, synthetic construct
  REGION 1..304: note = Sgp130 (D1, D2 and D3)
ELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRP

SEQ ID NO: 5 - protein, 50 aa, synthetic construct
  REGION 1..50: note = Linker (50aa)
GGGGSTRGQLHTQPEVEPQVDSPAPPRPSLQPHPRLLDHRDSVEQVAVGS

SEQ ID NO: 6 - protein, 210 aa, synthetic construct
  REGION 1..210: note = IL-11Ralpha (D2 and D3)
LGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTG

SEQ ID NO: 7 - protein, 8 aa, synthetic construct
  REGION 1..8: note = FLAG tag
DYKDDDDK

SEQ ID NO: 8 - protein, 33 aa, synthetic construct
  REGION 1..33: note = Linker (33aa)
GGGGSTRGSAGSGGSATGSGSAAGSGDSVRRGS

SEQ ID NO: 9 - DNA, 1809 bp, synthetic construct
  misc_feature 1..1809: note = Decoy IL-11 Receptor 1 (D11R1):
  source 1..1809: mol_type = other DNA
gaattcccgccgccaccatgctgaccctgcagacatggctggtgcaggccctgttcatcttcctgaccaccgagagcaccggcgagctgctggatccttgcggctacatcagccccgagagccctgtggtgcagctgcatagcaacttcaccgccgtgtgcgtgctgaaagaaaagtgcatggactacttccacgtgaacgccaactacatcgtgtggaaaacaaaccacttcaccatccccaaagagcagtacaccatcatcaaccggaccgccagcagcgtgaccttcaccgatatcgccagcctgaacatccagctgacctgcaacatcctgaccttcggccagctggaacagaacgtgtacggcatcacaatcatcagcggcctgccccccgagaagcccaagaacctgagctgcatcgtgaacgagggcaagaaaatgagatgcgagtgggacggcggcagagagacacacctggaaacaaacttcaccctgaagtccgagtgggccacccacaagttcgccgactgcaaggccaagagggacacccccaccagctgtaccgtggactacagcaccgtgtacttcgtgaacatcgaagtgtgggtggaagccgagaacgccctgggcaaagtgaccagcgaccacatcaacttcgaccctgtgtacaaagtgaagcccaaccccccccacaacctgagcgtgatcaacagcgaggaactgagcagcatcctgaagctgacatggaccaaccccagcatcaagtccgtgatcattctgaagtacaacatccagtaccggaccaaggacgccagcacctggtcccagatcccccctgaggataccgcctccacccggtccagcttcacagtgcaggacctgaagcctttcaccgagtacgtgttccggatcagatgcatgaaggaagatggcaagggctactggagcgattggagcgaggaagccagcggcatcacctacgaggacagacctggcggcggaggctctacaagaggccagctgcacactcagcccgaggtggaacctcaggtggactctcctgcccctcctagacctagcctgcagccccatcccagactgctggaccacagagacagcgtggaacaggtggccgtgggcagcctgggatatcctcctgctagacccgtggtgtcctgccaggccgccgactacgagaacttctcctgcacatggtcccccagccagatctccggcctgcctaccagatacctgaccagctaccggaagaaaaccgtgctgggcgccgacagccagagaagaagcccttctacaggcccctggccctgccctcaggatcctctgggagctgccagatgtgtggtgcacggcgccgagttttggagccagtacagaatcaacgtgaccgaagtgaaccccctgggcgccagcacaaggctgctggacgtgtccctgcagagcatcctgcggcctgatcctccacagggactgcgggtggaaagcgtgccaggctaccccagaaggctgagagcctcctggacataccccgctagctggccttgccagccccacttcctgctgaagttccggctgcagtaccggccagcccagcatccagcttggagcacagtggaacctgccggcctggaagaagtgatcacagacgccgtggccggactgcctcatgctgtgcgggtgtccgccagagactttctggatgccggaacctggtccacttggagcccagaggcttggggcacaccttccaccggcgactacaaggacgacgacgacaagtgataagctt

SEQ ID NO: 10 - DNA, 1758 bp, synthetic construct
  misc_feature 1..1758: note = Decoy IL-11 Receptor 2 (D11R2)
  source 1..1758: mol_type = other DNA
gaattcccgccgccaccatgctgaccctgcagacatggctggtgcaggccctgttcatcttcctgaccaccgagagcaccggcgagctgctggatccttgcggctacatcagccccgagagccctgtggtgcagctgcatagcaacttcaccgccgtgtgcgtgctgaaagaaaagtgcatggactacttccacgtgaacgccaactacatcgtgtggaaaacaaaccacttcaccatccccaaagagcagtacaccatcatcaaccggaccgccagcagcgtgaccttcaccgatatcgccagcctgaacatccagctgacctgcaacatcctgaccttcggccagctggaacagaacgtgtacggcatcacaatcatcagcggcctgccccccgagaagcccaagaacctgagctgcatcgtgaacgagggcaagaaaatgagatgcgagtgggacggcggcagagagacacacctggaaacaaacttcaccctgaagtccgagtgggccacccacaagttcgccgactgcaaggccaagagggacacccccaccagctgtaccgtggactacagcaccgtgtacttcgtgaacatcgaagtgtgggtggaagccgagaacgccctgggcaaagtgaccagcgaccacatcaacttcgaccctgtgtacaaagtgaagcccaaccccccccacaacctgagcgtgatcaacagcgaggaactgagcagcatcctgaagctgacatggaccaaccccagcatcaagtccgtgatcattctgaagtacaacatccagtaccggaccaaggacgccagcacctggtcccagatcccccctgaggataccgcctccacccggtccagcttcacagtgcaggacctgaagcctttcaccgagtacgtgttccggatcagatgcatgaaggaagatggcaagggctactggagcgattggagcgaggaagccagcggcatcacctacgaggacagacctggcggcggaggcagcacaagaggatctgctggaagcggcggaagcgccacaggctctggatctgcagctggcagcggagactctgtgcggagaggctctctgggctaccctcctgctagacccgtggtgtcttgtcaggccgccgactacgagaacttcagctgcacatggtcccccagccagatctccggcctgcctaccagatacctgaccagctaccggaagaaaaccgtgctgggcgccgacagccagagaagaagcccttctacaggcccctggccctgccctcaggatcctctgggagctgccagatgtgtggtgcacggcgccgagttttggagccagtacagaatcaacgtgaccgaagtgaaccccctgggcgccagcactagactgctggatgtgtccctgcagagcatcctgcggcccgatcctccacagggactgagagtggaaagcgtgcccggctaccccagaaggctgagagcctcctggacataccccgctagctggccttgccagccccacttcctgctgaagtttcggctgcagtaccggccagcccagcaccctgcttggagcacagtggaacctgccggcctggaagaagtgatcacagacgccgtggccggactgcctcatgctgtgcgggtgtccgccagagactttctggatgccggaacctggtccacttggagccctgaggcttggggcacaccttccaccggcgactacaaggacgacgacgacaagtgataagctt

SEQ ID NO: 11 - DNA, 66 bp, synthetic construct
  misc_feature 1..66: note = Leader sequence
  source 1..66: mol_type = other DNA
atgctgaccctgcagacatggctggtgcaggccctgttcatcttcctgaccaccgagagcaccggc

SEQ ID NO: 12 - DNA, 912 bp, synthetic construct
  misc_feature 1..912: note = Sgp130 (D1, D2 and D3)
  source 1..912: mol_type = other DNA
gagctgctggatccttgcggctacatcagccccgagagccctgtggtgcagctgcatagcaacttcaccgccgtgtgcgtgctgaaagaaaagtgcatggactacttccacgtgaacgccaactacatcgtgtggaaaacaaaccacttcaccatccccaaagagcagtacaccatcatcaaccggaccgccagcagcgtgaccttcaccgatatcgccagcctgaacatccagctgacctgcaacatcctgaccttcggccagctggaacagaacgtgtacggcatcacaatcatcagcggcctgccccccgagaagcccaagaacctgagctgcatcgtgaacgagggcaagaaaatgagatgcgagtgggacggcggcagagagacacacctggaaacaaacttcaccctgaagtccgagtgggccacccacaagttcgccgactgcaaggccaagagggacacccccaccagctgtaccgtggactacagcaccgtgtacttcgtgaacatcgaagtgtgggtggaagccgagaacgccctgggcaaagtgaccagcgaccacatcaacttcgaccctgtgtacaaagtgaagcccaaccccccccacaacctgagcgtgatcaacagcgaggaactgagcagcatcctgaagctgacatggaccaaccccagcatcaagtccgtgatcattctgaagtacaacatccagtaccggaccaaggacgccagcacctggtcccagatcccccctgaggataccgcctccacccggtccagcttcacagtgcaggacctgaagcctttcaccgagtacgtgttccggatcagatgcatgaaggaagatggcaagggctactggagcgattggagcgaggaagccagcggcatcacctacgaggacagacct

SEQ ID NO: 13 - DNA, 150 bp, synthetic construct
  misc_feature 1..150: note = Linker (50aa)
  source 1..150: mol_type = other DNA
ggcggcggaggctctacaagaggccagctgcacactcagcccgaggtggaacctcaggtggactctcctgcccctcctagacctagcctgcagccccatcccagactgctggaccacagagacagcgtggaacaggtggccgtgggcagc

SEQ ID NO: 14 - DNA, 630 bp, synthetic construct
  misc_feature 1..630: note = IL-11Ralpha (D2 and D3)
  source 1..630: mol_type = other DNA
ctgggatatcctcctgctagacccgtggtgtcctgccaggccgccgactacgagaacttctcctgcacatggtcccccagccagatctccggcctgcctaccagatacctgaccagctaccggaagaaaaccgtgctgggcgccgacagccagagaagaagcccttctacaggcccctggccctgccctcaggatcctctgggagctgccagatgtgtggtgcacggcgccgagttttggagccagtacagaatcaacgtgaccgaagtgaaccccctgggcgccagcacaaggctgctggacgtgtccctgcagagcatcctgcggcctgatcctccacagggactgcgggtggaaagcgtgccaggctaccccagaaggctgagagcctcctggacataccccgctagctggccttgccagccccacttcctgctgaagttccggctgcagtaccggccagcccagcatccagcttggagcacagtggaacctgccggcctggaagaagtgatcacagacgccgtggccggactgcctcatgctgtgcgggtgtccgccagagactttctggatgccggaacctggtccacttggagcccagaggcttggggcacaccttccaccggc

SEQ ID NO: 15 - DNA, 24 bp, synthetic construct
  misc_feature 1..24: note = FLAG tag
  source 1..24: mol_type = other DNA
gactacaaggacgacgacgacaag

SEQ ID NO: 16 - DNA, 99 bp, synthetic construct
  misc_feature 1..99: note = Linker (33aa)
  source 1..99: mol_type = other DNA
ggcggcggaggcagcacaagaggatctgctggaagcggcggaagcgccacaggctctggatctgcagctggcagcggagactctgtgcggagaggctct

SEQ ID NO: 17 - protein, 918 aa, synthetic construct
  REGION 1..918: note = Human gp130 (Genbank accession no. NP_002175.2)
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 18 - protein, 422 aa, synthetic construct
  REGION 1..422: note = Human IL-11Ralpha (Genbank accession no. NP_001136256.1; UniProt Q14626):
MSSSCSGLSRVLVAVATALVSASSPCPQAWGPPGVQYGQPGRSVKLCCPGVTAGDPVSWFRDGEPKLLQGPDSGLGHELVLAQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGTIPKEIPAWGQLHTQPEVEPQVDSPAPPRPSLQPHPRLLDHRDSVEQVAVLASLGILSFLGLVAGALALGLWLRLRRGGKDGSPKPGFLASVIPVDRRPGAPNL

SEQ ID NO: 19 - protein, 296 aa, synthetic construct
  REGION 1..296: note = The cytokine binding module of human gp130 corresponds to positions 26 to 321 of human gp130 (Genbank accession no. NP_002175.2)
DPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGIT

SEQ ID NO: 20 - protein, 206 aa, synthetic construct
  REGION 1..206: note = The cytokine binding module of human IL-11Ralpha corresponds to positions 112 to 317 of human IL-11Ralpha (Genbank accession no. NP_001136256.1; UniProt Q14626)
PPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPST

SEQ ID NO: 21 - DNA, 1575 bp, synthetic construct
  misc_feature 1..1575: note = DNA encoding IL-11:IL-11Ralpha fusion protein
  source 1..1575: mol_type = other DNA
gaattcccgccgccaccatgggctggtcctgcatcatcctgtttctggtggccacagccaccggcgtgcactctccacaggcttggggacctccaggcgtgcagtatggccagcctggcagatccgtgaagctgtgctgtcctggcgtgacagctggcgaccctgtgtcctggttcagagatggcgagcccaagctgctgcagggcccagattctggactgggccacgaactggtgctggcccaggccgattctaccgacgagggcacctacatctgccagaccctggatggcgccctgggcggaacagtgacactgcagctgggctaccctcccgccagacctgtggtgtcttgtcaggccgccgactacgagaacttcagctgcacatggtcccccagccagatcagcggcctgcccaccagatacctgaccagctaccggaagaaaaccgtgctgggcgccgacagccagagaagaagcccttctacaggcccctggccctgccctcaggatcctctgggagctgccagatgtgtggtgcacggcgccgagttctggtcccagtaccggatcaacgtgaccgaagtgaaccccctgggcgcctccacaagactgctggatgtgtccctgcagagcatcctgcggcccgatcctccacagggcctgagagtggaaagcgtgcccggctaccccagaaggctgagagccagctggacataccccgcctcttggccttgccagccccacttcctgctgaagtttcggctgcagtaccggccagcccagcaccctgcttggagcacagtggaacctgccggcctggaagaagtgatcacagacgccgtggccggactgcctcatgctgtgcgggtgtccgccagagactttctggatgccggcacctggtctacctggtccccagaagcctggggcacaccttctactggcggacctgctggacagtctggcggaggcggaggaagtggcggaggatcagggggaggatctgtgcctggacctcctccaggaccccctagagtgtccccagatcctagggccgagctggactctaccgtgctgctgaccagatccctgctggccgacacaaggcagctggctgcccagctgagagacaagttccccgccgacggcgaccacaacctggatagcctgcctaccctggccatgtctgctggcgcactgggggctctgcagctgcctggggtgctgactagactgagagccgacctgctgagctacctgcggcatgtgcagtggctgagaagggctggcggcagcagcctgaaaaccctggaacctgagctgggcacactgcaggccagactggacagactgctgcgcagactgcagctgctgatgagcagactggctctgccccagcctcctcctgaccctcctgctcctccactggctcctccaagctctgcttggggcggaattagagccgcccacgccattctgggaggcctgcacctgacactggattgggcagtgcggggcctgctgctgctgaaaaccagactgcaccaccaccatcaccactgataagctt

SEQ ID NO: 22 - protein, 516 aa, synthetic construct
  REGION 1..516: note = Amino acid sequence of IL-11:IL-11Ralpha fusion protein
MGWSCIILFLVATATGVHSPQAWGPPGVQYGQPGRSVKLCCPGVTAGDPVSWFRDGEPKLLQGPDSGLGHELVLAQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGGPAGQSGGGGGSGGGSGGGSVPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRLHHHHHH

SEQ ID NO: 23 - protein, 595 aa, synthetic construct
  REGION 1..595: note = Mouse gp130 ECD
QLLEPCGYIYPEFPVVQRGSNFTAICVLKEACLQHYYVNASYIVWKTNHAAVPREQVTVINRTTSSVTFTDVVLPSVQLTCNILSFGQIEQNVYGVTMLSGFPPDKPTNLTCIVNEGKNMLCQWDPGRETYLETNYTLKSEWATEKFPDCQSKHGTSCMVSYMPTYYVNIEVWVEAENALGKVSSESINFDPVDKVKPTPPYNLSVTNSEELSSILKLSWVSSGLGGLLDLKSDIQYRTKDASTWIQVPLEDTMSPRTSFTVQDLKPFTEYVFRIRSIKDSGKGYWSDWSEEASGTTYEDRPSRPPSFWYKTNPSHGQEYRSVRLIWKALPLSEANGKILDYEVILTQSKSVSQTYTVTGTELTVNLTNDRYVASLAARNKVGKSAAAVLTIPSPHVTAAYSVVNLKAFPKDNLLWVEWTPPPKPVSKYILEWCVLSENAPCVEDWQQEDATVNRTHLRGRLLESKCYQITVTPVFATGPGGSESLKAYLKQAAPARGPTVRTKKVGKNEAVLAWDQIPVDDQNGFIRNYSISYRTSVGKEMVVHVDSSHTEYTLSSLSSDTLYMVRMAAYTDEGGKDGPEFTFTTPKFAQGEIE

SEQ ID NO: 24 - protein, 597 aa, synthetic construct
  REGION 1..597: note = Human gp130 ECD
ELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIE

SEQ ID NO: 25 - protein, 294 aa, synthetic construct
  REGION 1..294: note = Mouse gp130 D1-D3
EPCGYIYPEFPVVQRGSNFTAICVLKEACLQHYYVNASYIVWKTNHAAVPREQVTVINRTTSSVTFTDVVLPSVQLTCNILSFGQIEQNVYGVTMLSGFPPDKPTNLTCIVNEGKNMLCQWDPGRETYLETNYTLKSEWATEKFPDCQSKHGTSCMVSYMPTYYVNIEVWVEAENALGKVSSESINFDPVDKVKPTPPYNLSVTNSEELSSILKLSWVSSGLGGLLDLKSDIQYRTKDASTWIQVPLEDTMSPRTSFTVQDLKPFTEYVFRIRSIKDSGKGYWSDWSEEASGTT

SEQ ID NO: 26 - protein, 350 aa, synthetic construct
  REGION 1..350: note = Mouse IL-11Ra ECD
SSPCPQAWGPPGVQYGQPGRPVMLCCPGVSAGTPVSWFRDGDSRLLQGPDSGLGHRLVLAQVDSPDEGTYVCQTLDGVSGGMVTLKLGFPPARPEVSCQAVDYENFSCTWSPGQVSGLPTRYLTSYRKKTLPGAESQRESPSTGPWPCPQDPLEASRCVVHGAEFWSEYRINVTEVNPLGASTCLLDVRLQSILRPDPPQGLRVESVPGYPRRLHASWTYPASWRRQPHFLLKFRLQYRPAQHPAWSTVEPIGLEEVITDAVAGLPHAVRVSARDFLDAGTWSAWSPEAWGTPSTGPLQDEIPDWSQGHGQQLEAVVAQEDSPAPARPSLQPDPRPLDHRDPLEQVAVLA

SEQ ID NO: 27 - protein, 348 aa, synthetic construct
  REGION 1..348: note = Human IL-11Ra ECD
SSPCPQAWGPPGVQYGQPGRSVKLCCPGVTAGDPVSWFRDGEPKLLQGPDSGLGHELVLAQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGTIPKEIPAWGQLHTQPEVEPQVDSPAPPRPSLQPHPRLLDHRDSVEQVAVLA

SEQ ID NO: 28 - protein, 206 aa, synthetic construct
  REGION 1..206: note = Mouse IL-11Ra D2-D3
PPARPEVSCQAVDYENFSCTWSPGQVSGLPTRYLTSYRKKTLPGAESQRESPSTGPWPCPQDPLEASRCVVHGAEFWSEYRINVTEVNPLGASTCLLDVRLQSILRPDPPQGLRVESVPGYPRRLHASWTYPASWRRQPHFLLKFRLQYRPAQHPAWSTVEPIGLEEVITDAVAGLPHAVRVSARDFLDAGTWSAWSPEAWGTPST